(19) United States
(12) Patent Application Publication
(10) Pub. No.: US 2026/0191774 A1
(43) Pub. Date: Jul. 9, 2026

(54) CHEWABLE COMPOSITIONS

(71) Applicant: Harmony Foods LLC, Santa Cruz, CA (US)

(72) Inventors: Mark Brian Jones, Cleveland, NC (US); Andrew Wassel, Mooresville, NC (US); Perry Nga, Dublin, CA (US); Michael Alan Simpson, Mooresville, NC (US); Jaimie L. Duensing, Kemersville, NC (US); Rachel Leah Hadley, Davidson, NC (US); Dylan Patrick Corry, Santa Cruz, CA (US)

(21) Appl. No.: 19/443,984

(22) Filed: Jan. 8, 2026

Related U.S. Application Data
(60) Provisional application No. 63/743,025, filed on Jan. 8, 2025.

(51) Int. Cl. A61K 9/00; A61K 31/439; A61K 31/4402; A61K 31/625; A61K 33/10; A61K 47/26; A61K 47/36

(52) U.S. Cl. CPC A61K 9/0056; A61K 31/439; A61K 31/4402; A61K 31/625; A61K 33/10; A61K 47/26; A61K 47/36

(57) ABSTRACT

Chewable compositions having a Fast Melt texture are provided. The compositions include a primary ingredient. The primary ingredient may be an active pharmaceutical ingredient, dietary supplement, or food. The compositions may include a hydrocolloid present at about 0.5% to about 15% by weight; and a bulk matrix present at about 65% to about 99.5% by weight that contains sugars or sugar alcohols and includes grained particles. The bulk matrix may include reducing sugars, non-reducing sugars, or combinations thereof, with the ratio of non-reducing to reducing sugars selected to control graining, hardness, and dissolving behavior. The compositions may include emulsifiers, sweeteners, flavors, colorants, stabilizers, buffering agents, or insoluble solids such as calcium carbonate. Methods of making the chewable compositions are also provided, including forming a hydrocolloid gel matrix, combining the matrix with a primary ingredient, heating the mixture, forming chewable units, and aging the units for 2-4 weeks to develop the Fast Melt texture.

### CROSS-REFERENCE TO RELATED APPLICATIONS

This application claims priority to U.S. Provisional Application No. 63/743,025 filed Jan. 8, 2025, the entire contents of which are incorporated by reference.

### FIELD OF THE DISCLOSURE

The present disclosure pertains to methods, manufacturing systems and processes, compositions, products, and methods of use related to chewable compositions that contain one or more primary ingredients. The primary ingredient may be an acid-sensitive active ingredients, active with an acid-sensitive coating, or acid-sensitive complex.

### BACKGROUND

Chewable compositions include chewable tablets or gummies. Traditional dosage forms include standard tablets, capsules, and liquid medications. Chewable dosage pharmaceutical forms have been developed to offer distinct advantages over traditional dosage forms. These advantages are driven by factors like patient compliance, ease of administration, bioavailability and patient demographics. They are especially beneficial for children, elderly patients, and individuals with dysphagia due to their palatability, convenience, and the elimination of the need for water. Additionally, chewable compositions can enhance bioavailability through faster dissolution and pre-gastric absorption while also reducing gastrointestinal irritation. Formulating these medications requires careful consideration of taste masking, texture, stability, and dosage uniformity to ensure efficacy and patient acceptability.

Chewable compositions, also referred to as gummies, pastilles, chewable gels, soft chewable tablets, or chewable bars, have become popular tools for providing dietary supplements and various medications in an easy-to-ingest vehicle. However, for effective delivery of many active ingredients, there are significant unmet needs to be overcome, including providing dosage forms with favorable palatability and texture to continuously improve patient compliance. It is with these observations, among others, that aspects of the present disclosure were conceived.

### BRIEF SUMMARY

The present disclosure is directed to a chewable composition. The chewable composition may have a Fast Melt texture. In some aspects, the chewable composition may include a primary ingredient selected from the group consisting of an active pharmaceutical ingredient, a dietary supplement, a food, and any combination thereof, a hydrocolloid, a bulk matrix, and water. In some aspects, the primary ingredient may be present in the chewable composition in an amount from about 0.05% to about 35% by weight. In some aspects, the hydrocolloid may be present in an amount from about 0.5% to about 15% by weight. In some aspects, the bulk matrix may be present in an amount from about 65% to about 99% by weight. In some other aspects, the bulk matrix composition may include grained particles.

The present disclosure is further directed to a composition for oral administration. The composition may have a Fast Melt texture. In some aspects, the composition may include a primary ingredient selected from the group consisting of an active pharmaceutical ingredient, a dietary supplement, a food, and any combination thereof, a hydrocolloid, a bulk matrix, and water. In some aspects, the primary ingredient may be present in the chewable composition in an amount from about 0.05% to about 35% by weight. In some aspects, the hydrocolloid may be present in an amount from about 0.5% to about 15% by weight. In some aspects, the bulk matrix may be present in an amount from about 65% to about 99% by weight. In some other aspects, the bulk matrix composition may include grained particles

The present disclosure is further directed to a method of producing a chewable composition. The method includes combining a hydrocolloid and one or more bulking agents to form a gel matrix, combining the gel matrix with a primary ingredient to form a mixture, heating the mixture to a temperature of at least 70° C., forming the mixture into chewable units, and allowing the chewable units to age for a period of 2 to 4 weeks to form the chewable composition.

### BRIEF DESCRIPTION OF THE FIGURES

FIG. 1 depicts a cross-section of an exemplary chewable composition having a plurality of active ingredients according to some embodiments of the present disclosure.

FIG. 2A is a categorical scatter plot illustrating rubberiness values assigned by trained sensory panelists for chewable compositions classified as Non-Fast Melt (0) and Fast Melt (1), according to some embodiments of the present disclosure. Each data point represents the mean for one sample using a standardized numeric scale.

FIG. 2B is a categorical scatter plot illustrating dissolvability values assigned by trained sensory panelists for chewable compositions classified as Non-Fast Melt (0) and Fast Melt (1), according to some embodiments of the present disclosure. Each data point represents the mean for one sample using a standardized numeric scale.

FIG. 2C is a categorical scatter plot illustrating toothpull values assigned by trained sensory panelists for chewable compositions classified as Non-Fast Melt (0) and Fast Melt (1), according to some embodiments of the present disclosure. Each data point represents the mean for one sample using a standardized numeric scale.

FIG. 2D is a categorical scatter plot illustrating hardness values obtained by Texture Profile Analysis (TPA) for chewable compositions classified as Non-Fast Melt (0) and Fast Melt (1), according to some embodiments of the present disclosure. Each data point represents the mean for one sample.

FIG. 2E is a categorical scatter plot illustrating cohesion values obtained by Texture Profile Analysis (TPA) for chewable compositions classified as Non-Fast Melt (0) and Fast Melt (1), according to some embodiments of the present disclosure. Each data point represents the mean for one sample.

FIG. 2F is a bar plot illustrating comparative hardness values obtained by Texture Profile Analysis (TPA) for a plurality of chewable compositions, including Fast Melt formulations and conventional gummy products, according to some embodiments of the present disclosure. Each bar represents an average hardness value for the corresponding sample, with error bars indicating measurement variability.

FIG. 2G is a bar plot illustrating comparative cohesion values obtained by Texture Profile Analysis (TPA) for a plurality of chewable compositions, including Fast Melt formulations and conventional gummy products, according to some embodiments of the present disclosure. Each bar represents an average cohesion value for the corresponding sample, with error bars indicating measurement variability.

FIG. 2H illustrates a cluster analysis of chewable compositions based on texture and sensory performance parameters, using average linkage hierarchical clustering with Euclidean distance, according to some embodiments of the present disclosure. The dendrogram groups products according to similarity in measured attributes, revealing clear separation between Fast Melt compositions and conventional gummy formulations.

FIG. 3A is a bar graph illustrating water activity values measured for a Fast Melt chewable composition over a three-month storage period, according to some embodiments of the present disclosure. Measurements were obtained at multiple timepoints, including initial production and subsequent storage intervals.

FIG. 3B is a bar graph illustrating average hardness values obtained by Texture Profile Analysis (TPA) for a Fast Melt chewable composition over a three-month storage period, according to some embodiments of the present disclosure. Measurements were taken at multiple timepoints following manufacture.

FIG. 3C is a bar graph illustrating average cohesion values obtained by Texture Profile Analysis (TPA) for a Fast Melt chewable composition over a three-month storage period, according to some embodiments of the present disclosure. Measurements were collected at multiple timepoints following manufacture.

FIG. 4A illustrates representative Texture Profile Analysis (TPA) force-time curves for a Fast Melt 1 at timepoint T0, according to some embodiments of the present disclosure.

FIG. 4B illustrates representative Texture Profile Analysis (TPA) force-time curves for a Fast Melt 1 at timepoint one week, according to some embodiments of the present disclosure.

FIG. 4C illustrates representative Texture Profile Analysis (TPA) force-time curves for a Fast Melt 1 at timepoint one month, according to some embodiments of the present disclosure.

FIG. 4D illustrates representative Texture Profile Analysis (TPA) force-time curves for a Fast Melt 1 at timepoint two months, according to some embodiments of the present disclosure.

FIG. 4E illustrates representative Texture Profile Analysis (TPA) force-time curves for a Fast Melt 1 at timepoint three months, according to some embodiments of the present disclosure.

FIG. 5A illustrates representative amplitude sweep rheology data comparing a Fast Melt chewable composition and a conventional gummy composition, according to some embodiments of the present disclosure. Storage modulus (G′) and loss modulus (G″) are plotted as a function of applied shear strain. The Fast Melt composition exhibits a lower Linear Viscoelastic Range (LVER) and earlier structural breakdown relative to the gummy composition, as evidenced by a decrease in G′ and a pronounced increase in G″ at elevated strain. The gummy composition maintains a broader LVER and higher resistance to deformation. The observed G″ increase at high strain indicates fracturing of the Fast Melt structure and transition to flow, consistent with reduced cohesiveness and rapid melt-in-mouth behavior.

FIG. 5B illustrates amplitude sweep rheology data for multiple chewable compositions, including Fast Melt formulations and conventional gummy products, according to some embodiments of the present disclosure. Storage modulus (G′) and loss modulus (G″) are plotted as a function of applied shear strain. The Fast Melt compositions exhibit lower Linear Viscoelastic Ranges (LVERs) and earlier structural breakdown relative to conventional gummy compositions, as evidenced by reductions in G′ and increases in G″ at elevated strain. In contrast, gummy compositions maintain higher moduli and broader LVERs prior to structural failure. The data demonstrate distinct rheological responses between Fast Melt and gummy formulations, supporting the classification of Fast Melt compositions as a mechanically fragile, rapidly disintegrating texture class.

FIG. 5C illustrates normalized thixotropic recovery profiles for Fast Melt chewable compositions and conventional gummy compositions, according to some embodiments of the present disclosure. Storage modulus recovery is plotted as a function of time following a high-strain interval used to disrupt the gel structure. Fast Melt compositions exhibit low percent recovery over the recovery interval, indicating limited structural regeneration after shear. In contrast, gummy compositions exhibit substantially higher percent recovery, reflecting rapid and extensive structural rebuilding. The distinct recovery profiles demonstrate differences in thixotropic behavior between Fast Melt and gummy formulations and correlate with reduced cohesiveness and rapid melt-in-mouth performance of Fast Melt compositions.

FIG. 5D illustrates final thixotropic percent recovery values for a plurality of chewable compositions following a three-interval thixotropy test, according to some embodiments of the present disclosure. Each bar represents the percent recovery of storage modulus (G′) measured after structural disruption relative to the pre-shear state.

FIG. 6A illustrates water activity measurements for multiple chewable compositions over a defined storage period, according to some embodiments of the present disclosure. Water activity values are plotted as a function of storage time for Fast Melt compositions and conventional gummy compositions. Fast Melt compositions maintain water activity within a controlled range over time, comparable to or lower than conventional gummy products, indicating moisture stability and suitability for long-term storage and microbial safety.

FIG. 6B illustrates hardness values obtained by Texture Profile Analysis (TPA) for multiple chewable compositions as a function of storage time, according to some embodiments of the present disclosure. Hardness measurements for Fast Melt compositions and conventional gummy compositions are plotted over the storage period.

FIG. 6C illustrates cohesion values obtained by Texture Profile Analysis (TPA) for multiple chewable compositions as a function of storage time, according to some embodiments of the present disclosure. Cohesion measurements for Fast Melt compositions and conventional gummy compositions are plotted over the storage period.

FIG. 7A is a bar graph illustrating average hardness values obtained by Texture Profile Analysis (TPA) for high-pH calcium carbonate chewable compositions formulated as a Non-Fast Melt product and a Fast Melt product, according to some embodiments of the present disclosure. Hardness values are plotted as a function of storage time.

FIG. 7B is a bar graph illustrating average cohesion values obtained by Texture Profile Analysis (TPA) for high-pH calcium carbonate chewable compositions formulated as a non-fast-melt product and a fast-melt product, according to some embodiments of the present disclosure. Cohesion values are plotted as a function of storage time.

FIG. 7C is a bar graph illustrating water activity measurements over time for high-pH calcium carbonate chewable compositions formulated as a Non-Fast Melt product and a Fast Melt product, according to some embodiments of the present disclosure. Water activity values are plotted at multiple storage timepoints for both formulations.

FIG. 7D is a force-time plot generated from TPA comparing the average compression behavior of Fast Melt product and Non-Fast Melt product at month three (M3), according to some embodiments of the present disclosure.

### DETAILED DESCRIPTION

The present disclosure is directed to a chewable composition having a Fast Melt texture. The chewable composition may include a primary ingredient, a hydrocolloid, a bulk matrix, and water. The chewable composition may be partially grained. The primary ingredient, the hydrocolloid, the bulk matrix, and the water may be uniformly distributed in the chewable composition. The chewable composition may further include insoluble solids.

Fast Melts are confectionery “gel” products that include a sugar or sugar-alcohol base (combining multiple forms of carbohydrate or hydrolyzed carbohydrate sugar-alcohols), and one or more hydrocolloids. The Fast Melt product form is designed to disintegrate in the mouth and disperse easily during chew down.

As defined herein, a composition having a “Fast Melt” texture is defined as a composition that has five properties defined further herein: a dissolvability of greater than 10; a rubberiness between 1 and 3, a toothpull of less than 2.5, a hardness of less than 10,000 g, and a cohesion between 0.05 and 0.3. It should be understood that the term “Fast Melt” does not necessarily refer to the speed at which the chewable compositions dissolve; although, the chewable compositions of the present disclosure may dissolve at a rate faster than similar compositions that do not have a Fast Melt texture.

As used herein, “dissolvability” refers to the rate at which the chewable composition breaks down and transitions from a solid state into a bolus or paste-like mass during mastication. Dissolvability reflects how quickly the product disintegrates in the mouth under normal chewing conditions, and is inversely related to the time required for the sample to lose its structural integrity. Higher dissolvability corresponds to faster breakdown, while lower dissolvability corresponds to slower disintegration. Dissolvability is measured using a 15-point scale, with low values representing slow or resistant breakdown, and high values representing rapid disintegration. Panelists are trained using a structured methodology consistent with the Spectrum Sensory Methodologies described in Sensory Evaluation Techniques, Sixth Edition (Meilgaard, Civille, Carr, Osdoba, 2025). Panelists are provided with a series of food product samples exhibiting a range of known textures, which serve as calibration standards for each evaluated attribute. During guided training sessions, panelists are instructed to associate each calibration standard with a corresponding numeric value and to apply these values when evaluating test samples. Panelists are trained to score the intensity of texture and mouthfeel attributes relevant to solid chewable compositions. Panelists are trained to ensure consistency, repeatability, and alignment with the overarching sensory-evaluation protocol referenced herein. Panelists assess the attribute by placing the sample between the molars and chewing at a consistent rate until the product exhibits a phase change into a cohesive bolus. The time and number of chews required to reach this transformation inform the dissolvability score, with Fast Melting materials yielding values above 10 on the 15-point scale. Calibration references include wheat cereal (slow dissolving), baked potato chips and crunchy snacks (intermediate), puffed cereals (high dissolvability), and cotton candy (very high dissolvability). The scale anchors range from slow breakdown (e.g., wheat cereal at 2.0) to extremely fast dissolution (e.g., cotton candy at 14.0).

The chewable compositions of the present disclosure have a dissolvability of greater than 10. For example, the chewable composition may have a dissolvability of 10.5 or more, 11 or more, 11.5 or more, 12 or more, 12.5 or more, or 13 or more. As another example, the chewable compositions of the present disclosure have a dissolvability from about 10 to about 11, about 10 to about 12, about 10 to about 13, about 10 to about 14, about 11 to about 14, about 12 to about 14, about 13 to about 14, about 11 to about 12, about 11 to about 13, or about 12 to about 13. As another example, the chewable composition may have a dissolvability of about 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, or about 14.

As used herein, “rubberiness” refers to the amount of elastic resistance or resilience perceived during mastication, specifically the resistance felt against the molars when the chewable composition is compressed and released. Rubberiness reflects the extent to which the sample rebounds, springs back, or maintains structural elasticity during chew-down. The attribute is assessed after several chews (typically between the third and sixth chews) and is recorded as the perceived intensity of elastic resistance on a numerical sensory scale. Rubberiness is quantified by Descriptive Analysis (DA) using a 15-point trained sensory scale, where lower values correspond to low resilience (soft, minimal rebound) and higher values correspond to highly elastic, gummy-like materials. Panelists are trained using a structured methodology consistent with the Spectrum Sensory Methodologies described in Sensory Evaluation Techniques, Sixth Edition (Meilgaard, Civille, Carr, Osdoba, 2025). Panelists are provided with a series of food product samples exhibiting a range of known textures, which serve as calibration standards for each evaluated attribute. During guided training sessions, panelists are instructed to associate each calibration standard with a corresponding numeric value and to apply these values when evaluating test samples. Panelists are trained to score the intensity of texture and mouthfeel attributes relevant to solid chewable compositions. Panelists are trained to ensure consistency, repeatability, and alignment with the overarching sensory-evaluation protocol referenced herein. Calibration standards anchor the scale, with marshmallow defined as the low end of resilience (e.g., ~1.0), string cheese and turkey sausage defining intermediate levels, and gummy candy and gummy bears defining high rubberiness values (e.g., 9-13). Fast Melt compositions may exhibit low rubberiness (approximately 1-3), indicating minimal elastic resistance relative to conventional gummies or chewable gels.

The chewable compositions may have a rubberiness from about 1 to about 3, such as from about 1 to about 1.5, about 1 to about 2, about 1 to about 2.5, about 1 to about 3, about 1.5 to about 3, about 2 to about 3, about 2.5 to about 3, or about 1.5 to about 2.5. As another example, the chewable compositions may have a rubberiness of about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3.

As used herein, “toothpull” refers to the force required to separate the upper and lower teeth during mastication of the chewable composition. Toothpull measures the degree of adhesion or stickiness the sample exhibits as it is compressed and released between the molars. Higher toothpull values indicate greater adhesion or “pull” between the teeth, while lower values indicate minimal adhesion and little to no sticking. Toothpull is quantified on a 15-point trained sensory scale, where lower values correspond to low stickiness (e.g., soft granola bar materials) and higher values correspond to strongly adhesive, gummy-like materials (e.g., certain gelatin- or starch-based candies). Panelists are trained using a structured methodology consistent with the Spectrum Sensory Methodologies described in Sensory Evaluation Techniques, Sixth Edition (Meilgaard, Civille, Carr, Osdoba, 2025). Panelists are provided with a series of food product samples exhibiting a range of known textures, which serve as calibration standards for each evaluated attribute. During guided training sessions, panelists are instructed to associate each calibration standard with a corresponding numeric value and to apply these values when evaluating test samples. Panelists are trained to score the intensity of texture and mouthfeel attributes relevant to solid chewable compositions. Panelists are trained to ensure consistency, repeatability, and alignment with the overarching sensory-evaluation protocol referenced herein. Panelists evaluate the attribute by placing the sample between the molars and chewing five times within approximately 10 seconds, then assessing the force required to pull the teeth apart during and immediately after compression. Chewable products that are soft, non-sticky, and quickly melting exhibit very low toothpull values (approximately 1-2.5), while traditional gummies, chewy candies, and elastic gel-based pieces exhibit significantly higher values.

The chewable compositions of the present disclosure have a toothpull of less than 2.5, less than 2, less than 1.5, or less than 1. For example, the chewable compositions of the present disclosure may have a toothpull from about 0.1 to about 0.5, about 0.1 to about 1, about 0.1 to about 1.5, about 0.1 to about 2, about 0.1 to about 2.5, about 0.5 to about 2.5, about 1 to about 2.5, about 1.5 to about 2.5, about 2 to about 2.5, about 1 to about 2, about 0.5 to about 2, or about 0.5 to about 1.5. As another example, the chewable compositions of the present disclosure may have a toothpull of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, or about 2.5.

As used herein, “hardness” refers to the maximum peak force required to compress the chewable composition during the first compression cycle of a Texture Profile Analysis (TPA) test. Hardness represents the initial firmness of the sample when subjected to a controlled deformation and is reported as the highest force value (in grams) recorded as the probe compresses the product to a predetermined strain level. Hardness reflects the resistance of the sample to deformation and correlates with the perceived firmness when the product is first bitten. Hardness is measured using a Micro Systems Texture Analyzer. During testing, the chewable composition is placed on a TA-90 metal platform, and the probe compresses the sample to either 25% or 50% strain, generating a force-time curve. The maximum force recorded during the first compression peak is reported as the hardness value. This measure differentiates Fast Melt compositions from conventional compressed tablets and from certain gummy products.

The chewable compositions of the present disclosure have a hardness of less than 10,000 g, such as less than 9,000 g, less than 8,000 g, less than 7,000 g, less than 6,000 g, or less than 5,000 g. As another example, the chewable compositions of the present disclosure may have a hardness from about 100 g to about 2,000 g, about 100 g to about 4,000 g, about 100 g to about 6,000 g, about 100 g to about 8,000 g, about 100 g to about 10,000 g, about 2,000 g to about 10,000 g, about 4,000 g to about 10,000 g, about 6,000 g to about 10,000 g, about 8,000 g to about 10,000 g, about 2,000 g to about 8,000 g, or about 4,000 g to about 6,000 g. As another example, the chewable compositions of the present disclosure have a hardness of about 100 g, 200 g, 300 g, 400 g, 500 g, 600 g, 700 g, 800 g, 900 g, 1,000 g, 1,500 g, 2,000 g, 2,500 g, 3,000 g, 3,500 g, 4,000 g, 4,500 g, 5,000 g, 5,500 g, 6,000 g, 6,500 g, 7,000 g, 7,500 g, 8,000 g, 8,500 g, 9,000 g, 9,500 g, or about 10,000 g.

As used herein, “cohesiveness” refers to the extent to which a chewable composition withstands a second deformation relative to a first deformation and is indicative of the internal structural integrity of the sample. Cohesiveness is a dimensionless ratio derived from Texture Profile Analysis (TPA) and reflects how well the product holds together during repeated deformation, such as during mastication. In a TPA test, the sample is subjected to two successive compression cycles using a texture analyzer, with a brief pause between the first and second compressions. The pause allows partial recovery of the sample structure prior to the second compression. This double-compression protocol generates two force-time curves corresponding to the first and second compressions and provides insight into how the product behaves when chewed multiple times. A product is cohesive when it adheres to itself under some compressive stress.

Cohesiveness is calculated as the ratio of the area of work (positive force area) during the second compression to the area of work during the first compression, as follows:

Cohesiveness=(positive force area during second compression)/(positive force area during first compression)

This ratio indicates how well a sample withstands multiple deformations. Lower cohesiveness indicates that the sample breaks down easily and rapidly loses structural integrity, consistent with Fast Melt compositions. Higher cohesiveness indicates that the sample retains structure upon repeated compression, characteristic of traditional gummies or more elastic gel systems.

The chewable compositions of the present disclosure have a cohesion from about 0.05 to about 0.3. For example, the chewable composition of the present disclosure may have a cohesion from about 0.05 to about 0.1, about 0.05 to about 0.15, about 0.05 to about 0.2, about 0.05 to about 0.25, about 0.05 to about 0.3, about 0.1 to about 0.3, about 0.15 to about 0.3, about 0.2 to about 0.3, about 0.25 to about 0.3, about 0.1 to about 0.2, or about 0.15 to about 0.25. As another example, the chewable composition of the present disclosure may have a cohesion of about 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, or about 0.3.

The chewable compositions of the present disclosure include a bulk matrix. The bulk matrix is defined by the presence of grained particles. The grained particles form over time during an aging process described further herein. The grained particles help to achieve the Fast Melt texture that define the chewable compositions of the present disclosure. Without wishing to be bound by theory, the grained particles may form as the result of a supersaturation of sugars or other bulking agents in the bulk matrix of the chewable composition and may be aided by inclusion of nucleation sites such as by the addition of insoluble solids.

The percent of the bulk matrix in the chewable composition may be from about 65% to about 99.5% by weight of the chewable composition. For example, the percent of the bulk matrix may be from about 65% to about 99%, from about 65% to about 98%, from about 65% to about 97%, from about 65% to about 96%, from about 65% to about 95%, from about 65% to about 94%, from about 65% to about 93%, from about 65% to about 92%, from about 65% to about 91%, from about 65% to about 90%, from about 65% to about 89%, from about 65% to about 88%, from about 65% to about 86%, from about 65% to about 85%, from about 65% to about 84%, from about 65% to about 83%, from about 65% to about 82%, from about 65% to about 81%, from about 65% to about 80%, from about 65% to about 79%, from about 65% to about 78%, from about 65% to about 76%, from about 65% to about 75%, from about 65% to about 74%, from about 65% to about 73%, from about 65% to about 72%, from about 65% to about 71%, from about 65% to about 70%, from about 65% to about 69%, from about 65% to about 68%, from about 65% to about 67%, from about 65% to about 66%, from about 67% to about 99.5%, from about 67% to about 98%, from about 67% to about 95%, from about 67% to about 90%, from about 67% to about 85%, from about 67% to about 80%, from about 67% to about 75%, from about 67% to about 70%, from about 67% to about 69%, from about 67% to about 68%, from about 68% to about 99%, from about 68% to about 97%, from about 68% to about 90%, from about 68% to about 88%, from about 68% to about 86%, from about 68% to about 84%, from about 68% to about 82%, from about 68% to about 80%, from about 68% to about 78%, from about 68% to about 76%, from about 68% to about 74%, from about 68% to about 72%, from about 68% to about 70%, from about 68% to about 69%, from about 69% to about 99.5%, from about 69% to about 97%, from about 69% to about 90%, from about 69% to about 88%, from about 69% to about 86%, from about 69% to about 84%, from about 69% to about 82%, from about 69% to about 80%, from about 69% to about 78%, from about 69% to about 76%, from about 69% to about 74%, from about 69% to about 72%, from about 69% to about 70%, from about 70% to about 99.5%, from about 70% to about 99%, from about 70% to about 98%, from about 70% to about 97%, from about 70% to about 95%, from about 70% to about 92%, from about 70% to about 90%, from about 70% to about 88%, from about 70% to about 85%, from about 70% to about 80%, from about 70% to about 72%, from about 71% to about 99.5%, from about 71% to about 99%, from about 71% to about 98%, from about 71% to about 97%, from about 71% to about 95%, from about 71% to about 92%, from about 71% to about 90%, from about 71% to about 88%, from about 71% to about 85%, from about 71% to about 80%, from about 71% to about 72%, from about 72% to about 99.5%, from about 72% to about 98%, from about 72% to about 95%, from about 72% to about 90%, from about 72% to about 85%, from about 72% to about 83%, from about 72% to about 80%, from about 72% to about 75%, from about 72% to about 73%, from about 73% to about 99.5%, from about 73% to about 98%, from about 73% to about 95%, from about 73% to about 90%, from about 73% to about 85%, from about 73% to about 83%, from about 73% to about 80%, from about 73% to about 75%, from about 73% to about 74%, from about 74% to about 99.5%, from about 74% to about 98%, from about 74% to about 95%, from about 74% to about 90%, from about 74% to about 85%, from about 74% to about 83%, from about 74% to about 80%, from about 74% to about 75%, from about 74% to about 75%, from about 75% to about 99.5%, from about 75% to about 99%, from about 75% to about 98%, from about 75% to about 97%, from about 75% to about 95%, from about 75% to about 92%, from about 75% to about 90%, from about 75% to about 88%, from about 75% to about 85%, from about 78% to about 99%, from about 78% to about 98%, from about 78% to about 95%, from about 78% to about 90%, from about 80% to about 99.5%, from about 80% to about 99%, from about 80% to about 98%, from about 80% to about 97%, from about 80% to about 95%, from about 80% to about 92%, from about 80% to about 90%, from about 82% to about 99%, from about 82% to about 98%, from about 82% to about 95%, from about 82% to about 90%, from about 85% to about 99.5%, from about 85% to about 99%, from about 85% to about 98%, from about 85% to about 97%, from about 85% to about 95%, from about 85% to about 92%, from about 85% to about 90%, from about 88% to about 99.5%, from about 88% to about 99%, from about 88% to about 98%, from about 88% to about 97%, from about 88% to about 95%, from about 90% to about 99.5%, from about 90% to about 99%, from about 90% to about 98%, from about 90% to about 97%, from about 90% to about 95%, from about 92% to about 99.5%, from about 92% to about 99%, from about 92% to about 98%, from about 92% to about 97%, from about 95% to about 99.5%, from about 95% to about 99%, from about 95% to about 98%, or from about 98% to about 99.5% by weight of the chewable composition.

The bulk matrix may include a sugar. The sugar may be a reducing sugar, a non-reducing sugar, or a combination thereof. In some embodiments, the bulk matrix may include one or more reducing sugars. Suitable reducing sugars include, but are not limited to, glucose (including L-glucose), fructose (including L-fructose), galactose, psicose, isomaltulose, tagatose, trehalose, sorbose, D-maltose (1,4-diglucose), raffinose, or combinations thereof. These materials may contribute to moisture retention, rapid softening, and enhanced meltability during mastication.

In some embodiments, the bulk matrix may include one or more non-reducing sugars. Non-reducing sugars may improve structural stability, reduce browning, and provide a smooth, crystalline, or semi-crystalline support phase. Exemplary non-reducing sugars include sucrose, maltose, trehalose, sorbose, and raffinose, as well as any combination thereof.

In some embodiments, the bulk matrix includes both a reducing sugar and a non-reducing sugar. The relative amounts of each may be selected to tailor the hardness, elasticity, dissolution profile, and overall texture of the chewable composition. In certain embodiments, the mass ratio of non-reducing sugar to reducing sugar ranges from about 90:10 to about 30:70. Ratios within this range have been found to produce a Fast Melt texture that softens rapidly in the mouth while maintaining sufficient mechanical integrity for handling.

In certain embodiments, the mass ratio of the non-reducing sugar to the reducing sugar in the bulk matrix may be from about 90:10 to about 30:70. For example, the mass ratio may be from about 90:10 to about 80:20, from about 85:15 to about 70:30, from about 80:20 to about 60:40, from about 75:25 to about 50:50, from about 70:30 to about 40:60, and from about 60:40 to about 30:70. In some embodiments, the mass ratio may be from about 85:15 to about 65:35, from about 80:20 to about 55:45, from about 75:25 to about 45:55, and from about 70:30 to about 50:50. In yet other embodiments, the mass ratio may be from about 90:10 to about 85:15, from about 85:15 to about 80:20, from about 80:20 to about 75:25, from about 75:25 to about 70:30, from about 70:30 to about 65:35, from about 65:35 to about 60:40, from about 60:40 to about 55:45, from about 55:45 to about 50:50, from about 50:50 to about 45:55, from about 45:55 to about 40:60, from about 40:60 to about 35:65, and from about 35:65 to about 30:70. In certain preferred embodiments, the mass ratio may be from about 80:20 to about 40:60, from about 75:25 to about 50:50, or from about 70:30 to about 60:40.

In additional embodiments, the bulk matrix may include one or more low-calorie bulking agents in addition to the sugar or as an alternative to the sugar. These components may partially or fully replace conventional nutritive sugars to reduce caloric content while maintaining desirable gel structure, sweetness, and textural characteristics.

Suitable low-calorie bulking agents include sugar alcohols (polyols). Non-limiting examples of polyols include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, and sugar alcohols or any other carbohydrates capable of being reduced which do not adversely affect the taste of the composition. When incorporated into the bulk matrix, such agents may provide controlled sweetness, reduced glycemic impact, and enhanced melt-in-mouth performance, while also contributing to moisture management and preventing excessive stickiness or toothpull.

The bulk matrix, whether comprised of reducing sugars, non-reducing sugars, low-calorie bulking agents, or mixtures thereof, may be formulated to modulate the chewable composition's rheological behavior, including cohesiveness, dissolvability, and rubberiness during chewing. By adjusting the carbohydrate system, the composition can achieve the desired Fast Melt texture profile characterized by low chew resistance, rapid disintegration, and minimal adhesion to the teeth.

The bulking agent may further or alternatively include a liquid sweetener and a solid sweetener. The phrases “liquid sweetener” and “solid sweetener” refer to the state of matter of the sweetener at the time the chewable composition is manufactured, which may not be the state of matter of the sweetener in the chewable composition. The liquid sweetener and the solid sweetener may each comprise any of the bulking agents described above in liquid form or solid form at the time the chewable composition is made, respectively. The amount of liquid sweetener in the chewable composition may range from about 0% to 70%. For example, the amount of liquid sweetener may range from about 0% to about 70%, about 5% to about 65%, about 5% to about 60%, about 5% to about 55%, about 5% to about 50%, about 5% to about 45%, about 10% to about 40%, about 15% to about 35%, or about 20% to about 30%. The amount of solid sweetener in the chewable composition may range from about 0% to 90%. For example, the amount of solid sweetener may range from about 0% to about 90%, about 0% to about 85%, about 0% to about 80%, about 0% to about 75%, about 0% to about 70%, about 0% to about 65%, about 0% to about 60%, about 0% to about 55%, about 0% to about 50%, about 5% to about 45%, about 10% to about 40%, about 15% to about 35%, or about 20% to about 30%. Examples of liquid sweeteners include but are not limited to honey, agave nectar, maple syrup, corn syrup, molasses, brown rice syrup, barley malt syrup, coconut nectar, or stevia extract. Examples of solid sweeteners include but are not limited to granulated sugar, brown sugar, powdered sugar, raw sugar, coconut sugar, muscovado sugar, turbinado sugar, or xylitol. In some aspects, the sweetener may have as raw materials both a liquid syrup such as corn syrup and a solid carbohydrate such as sucrose. The solid carbohydrate may constitute at least about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, or about 93% of the sweetener.

In some aspects, the ratio of the non-syrup sweeteners or solid sugar sweeteners to syrup may range from about 60:40 to about 95:5. For example, the ratio may be 65:35, 68:32, 70:30, 75:25, 80:20, 85:15, 90:10, or 93:7. Surprisingly, benefits such as enhanced performance benefits for a chewable composition matrix at elevated pH may be achieved by elevating the ratio of sugar to corn syrup relative to commonly used ranges. Enhanced performance benefits include but are not limited to organoleptic properties that may help with compliance of active administration. For example, contrary to the common belief that the sugar to corn syrup ratio should not exceed 65:35 (65 parts sugar to 35 corn syrup on a dry basis) or 60:40 less sugar crystals form in the chewable composition, higher ratios such as 70:30 or 75:25 may be used and that such elevated ranges of the sugar-corn syrup ratio may enhance the performance of a chewable composition matrix.

In some aspects, a wide variety of sweeteners may included in the bulk matrix such as sucrose, fructose, honey, fruit products, agave, maple syrup, stevia, allulose, monk fruit extract, xylitol, high fructose corn syrup, dextrose, saccharin, maltodextrin, aspartame, potassium acesulfame (acesulfame-K or Ace-K), sucralose, neotame, advantame, neohesperidin dihydrochalcone, and the like. Fruit products include but are not limited to fruit extracts, fruit slurries, dried fruit, fruit juice, and the like.

In some aspects, the pH of the chewable composition matrix may be from about 3 to about 11, about 3.5 to about 10.5, about 4 to about 10, about 4.5 to about 9.5, about 5 to about 8.5, about 5.5 to about 8, about 6 to about 7.5, about 6.5 to about 7, about 7 to about 11, about 7.5 to about 10.5, about 8.0 to 10, about 8.5 to about 9.5, or about 8.7 to about 9.0. In some embodiments, the pH of the chewable composition may be at least 5.

The chewable composition further includes a hydrocolloid. The hydrocolloid comprises carrageenan, gelatin, pectin, agar, starch (e.g., corn starch, tapioca starch, arrowroot starch, potato starch, wheat starch, yam starch, mung bean starch, rice starch, bracken starch, and modified starch, and the like), locust beam gum, konjac, gum Arabic, sago, or any combination thereof. In some embodiments, the chewable composition matrix may be substantially free of pectin, gelatin, or both. In some embodiments, the chewable composition matrix may have relatively low amounts (e.g., less than 2% by weight) of pectin, gelatin, or both.

In some embodiments, the hydrocolloid includes carrageenan. Any suitable type of carrageenan may be selected such as kappa, lambda, and iota carrageenans or mixtures thereof. In one aspect, a carrageenan such as mixture of kappa, lambda, and iota carrageenan (with ratios of 60:10:30, 70:0:30 or 50:20:30 parts per hundred of kappa, lambda, and iota, respectively) in the form of a dry powder may be combined with water at a temperature below 50° C. and dispersed gently before being combined with additional ingredients and heated as the mixture is heated to over 80° C. Potassium ions may be present, which tend to produce stiff gels from kappa carrageenan.

Unexpectedly, the disclosed chewable compositions with carrageenan exhibit improved palatability, stability, release profile, and performance of the primary ingredient. The chewable compositions may include carrageenan. The carrageenan may be partially hydrated. For example, the amount of water may be at least about 10%, about 20%, about 30%, about 40%, or less than about 50% less than the water needed for full hydration), and then blended with the other ingredients to form the aqueous matrix solution. In doing this, the time between the addition of water to the carrageenan powder and the combination with additional moisture, sweetener, and other ingredients to form the aqueous matrix solution may be relatively brief. For example, the time may be less than 15 minutes, 10 minutes, 5 minutes, 2 minutes, 1 minutes, or 30 seconds. The time may include the time for blending all or at least 30% of the sweetener and moisture to be used at substantially the same time with the hydrocolloid to reduce further hydration of the carrageenan, or to reduce the migration of moisture from hydrated portions of the carrageenan to less hydrated portions. After the hydrocolloid has been combined into the aqueous matrix solution, the pH of the aqueous matrix solution may change over time during cooking, but may initially be above about 3, about 4, about 5, about 6, about 7, about 8, or about 9. In some embodiments, the pH may range from about 6 to about 11, about 6.5 to about 10.5, about 7.0 to about 10, about 7.5 to about 9.5, or about 8 to about 9. Temperatures may be elevated thereafter to complete the cooking of the aqueous matrix solution to prepare the chewable composition matrix.

In one aspect, a hydrocolloid such as a dry carrageenan (powdered carrageenan) may be dispersed in water having a pH from about 3 to about 10.5. In another aspect, less water may be provided than is required for full hydration of carrageenan, such as from about 5% to about 40% less or from about 5% to about 20% less water.

The percent of hydrocolloid in the chewable composition may be from about 0.5% to about 15% by weight of the chewable composition. For example, the amount of the hydrocolloid may be from about 0.5% to about 14%, from about 0.5% to about 12%, from about 0.5% to about 10%, from about 0.5% to about 8%, from about 0.5% to about 6%, from about 0.50% to about 5%, from about 0.50% to about 1%, from about 1% to about 15%, from about 1% to about 14%, from about 1% to about 12%, from about 1% to about 10%, from about 1% to about 8%, from about 1% to about 5%, from about 1% to about 2%, from about 2% to about 15%, from about 2% to about 12%, from about 2% to about 10%, from about 2% to about 8%, from about 2% to about 5%, from about 2% to about 3%, from about 3% to about 15%, from about 3% to about 12%, from about 3% to about 10%, from about 3% to about 8%, from about 3% to about 5%, from about 3% to about 4%, from about 4% to about 15%, from about 4% to about 12%, from about 4% to about 10%, from about 4% to about 8%, from about 4% to about 5%, from about 5% to about 15%, from about 5% to about 12%, from about 5% to about 10%, from about 5% to about 8%, from about 5% to about 6%, from about 6% to about 15%, from about 6% to about 12%, from about 6% to about 10%, from about 6% to about 8%, from about 6% to about 7%, from about 7% to about 15%, from about 7% to about 12%, from about 7% to about 10%, from about 7% to about 9%, from about 7% to about 8%, from about 8% to about 15%, from about 8% to about 10%, from about 8% to about 9%, from about 9% to about 15%, from about 9% to about 12%, from about 9% to about 10%, from about 10% to about 15%, from about 10% to about 12%, from about 12% to about 15%, from about 12% to about 14%, or from about 14% to about 15% by weight of the chewable composition. As another example, the hydrocolloid may be present in the chewable composition in an amount of about 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 1%. 5%, 12%, 12.5%, 13%, 13.5%, 14%, 14.5%, or about 15% by weight of the chewable composition.

The chewable composition further includes a primary ingredient. The primary ingredient may include one or more active pharmaceutical ingredients (APIs), dietary supplements, food substances, or combinations thereof. The secondary or tertiary ingredient may include one or more active pharmaceutical ingredients (APIs), dietary supplements, food substances, or combinations thereof. In some embodiments, the chewable composition may further include a second primary ingredient (i.e., a secondary ingredient) that is different from the primary ingredient. In some embodiments, the chewable composition may further include a third primary ingredient (i.e., a tertiary ingredient) that is different from the first primary ingredient and the second primary ingredient. The secondary or tertiary ingredient may include one or more active pharmaceutical ingredients (APIs), dietary supplements, food substances, or combinations thereof.

The primary ingredient may include an active pharmaceutical ingredient. Active pharmaceutical ingredients may include compounds that may undergo chemical change during the manufacture of the composition (e.g., via interaction with moisture, acidulants, buffering agents, sweeteners, gelling agents, or due to thermal effects) and be present in the final composition in a modified form intended to furnish an activity or effect. For example, the active pharmaceutical ingredient may include but is not limited to aspirin, ibuprofen, and/or guaifenesin. Aspirin may degrade in the presence of moisture. Ibuprofen and guaifenesin may react thermally because of their low melting point.

APIs may include analgesics, antibiotics, anticoagulants and antiplatelet agents, antidiabetic agents, antiemetics, antihistamines, anti-asthmatic medications, antiarrhythmics, anticholinergics, anticholinesterase inhibitors, anticonvulsants, antidepressants, antidiabetic agents, antihistamines, antihypertensives (ACE inhibitors, alpha blockers, angiotensin II receptor blockers, beta blockers, calcium channel blockers, central alpha agonists, diuretics, and the like), anti-inflammatory drugs, antipsychotics, antipyretics, appetite suppressants, bronchodilators, chemotherapy compounds, cholesterol level medications (bile acid sequestrants, cholesterol absorption inhibitor such as ezetimibe, fibrates, PCSK9 inhibitors, statins, angiotensin II receptor blockers, beta blockers, calcium channel blockers, central alpha agonists, and the like), diuretics, expectorants, hormonal medications, laxatives, sedatives, stimulants, vasodilators, anxiolytics, mood stabilizers, antipsychotics, antidepressants and antipsychotics, and the like.

In some embodiments, the active pharmaceutical ingredient may include antacids, laxatives, stomach relief medications, antiflatulents, antidiarrheals, antihistamines, antitussives, allergy medications, antibiotics, proteins, peptides, or combinations thereof.

Aspects of the present disclosure may be equally applicable to compositions where the primary ingredient is selected from the group comprising of anti-inflammatory actives, coronary dilators, cerebral dilators, peripheral vasodilators, anti-infectives, psychotropics, antimanics, stimulants, gastro-intestinal sedatives, antidiarrheal primary ingredients, anti-anginal drugs, vasodilators, anti-hypertensive drugs, vasoconstrictors and anti-migraine treatment actives, antibiotics, tranquilizers, antipsychotics, antitumor drugs, anticoagulants and antithrombotic drugs, hypnotics, sedatives, anti-emetics, antinauseants, anticonvulsants, neuromuscular drugs, hyper- and hypoglycemic agents, thyroid and antithyroid preparations, diuretics, antispasmodics, uterine relaxants, nutritional additives, anti-obesity drugs, anabolic drugs, erythropoietic drugs, antiasthmatics, expectorants, cough suppressants, mucolytics, anti-hyperuricemia drugs, antidepressants, and the like, and combinations of the foregoing.

In some embodiments, the API may include an acid-sensitive active ingredient, active with an acid-sensitive coating, and/or acid-sensitive complex.

In some aspects, the at least one acid-sensitive active ingredient includes antacids, laxatives, stomach relief medications, antidiarrheals, allergy medications, acid-labile antibiotics, antiflatulents, acid-sensitive proteins and peptides, alkaline active pharmaceutical ingredients (APIs), or combinations thereof. For example, the acid-sensitive ingredient may include calcium hydroxide, bismuth subsalicylate, magnesium hydroxide, aluminum hydroxide, other salts of calcium and magnesium, various carbonate and bicarbonate compounds, Calcium Carbonate, cetylpyridinium chloride (CET), hydrogen blockers such as famotidine and ranitidine, proton pump inhibitors (PPI) such as Omeprazole and Lansoprazole, diphenylmethylpiperazine antihistamines such as cetirizine, cyclizine, and hydroxzine, antiflatulents such as simethicone.

In some aspects, the at least one acid-sensitive coating includes enteric coatings and reverse enteric coatings. Suitable enteric coatings include but are not limited to cellulose acetate phthalate, methacrylic acid copolymers and hydroxypropyl methylcellulose phthalate. Reverse enteric coating materials are pH-responsive polymers that are applied to oral dosage forms to mask taste and prevent dissolution in the mouth but release the formulation in the stomach. Some examples of reverse enteric coating materials include Eudragit and Poly[(2-vinylpyridine)-co-(butyl methacrylate)] copolymers. In some aspects, the at least one acid-sensitive complex may be cyclodextrin or ion exchange resin-complexed actives. Examples of ion exchange resins include sodium polystyrene sulfonate, colestipol, and cholestyramine. In some embodiments, the at least one acid-sensitive active ingredient, active with an acid-sensitive coating, or acid-sensitive complex may be unstable at a pH in at least portion of the pH range from 3 to 5.5. In some embodiments, the acid-sensitive active ingredient, active with an acid-sensitive coating, or acid-sensitive complex may have a pH greater than 7 or maybe stable at a pH greater than 7, while being unstable below a pH of 5.5.

The amount of the at least one acid-sensitive active ingredient, active with an acid-sensitive coating, or acid-sensitive complex in the chewable composition may range from about 5% to about 70%. For example, the amount of the at least one acid-sensitive active ingredient, active with an acid-sensitive coating, or acid-sensitive complex may range from about 10% to about 65%, about 15% to about 60%, about 20% to about 55%, about 25% to about 50%, about 30% to about 45%, or about 35% to about 40%. In some embodiments, the at least one acid-sensitive active may constitute at least 7% of the chewable composition. Antacids, for example, may include any pharmaceutically acceptable antacids such as salts of calcium, magnesium, or aluminum. Examples of the salts of calcium, magnesium, or aluminum include but are not limited to calcium carbonate, magnesium carbonate, magnesium hydroxide, magnesium trisilicate, or aluminum hydroxide. The antacids may further include sodium bicarbonate, alginate, and the like. In some embodiments, the antacids may be present at a dose ranging from about 10 mg to about 2000 mg per chewable composition. For example, the dose may range from about 10 mg to about 1950 mg, about 50 mg to about 1900 mg, about 100 mg to about 1850 mg, about 150 mg to about 1950 mg, about 200 mg to about 1900 mg, about 250 mg to about 1850 mg, about 300 mg to about 1800 mg, about 350 mg to about 1750 mg, about 400 mg to about 1700 mg, about 450 mg to about 1650 mg, about 500 mg to about 1600 mg, about 550 mg to about 1550 mg, about 600 mg to about 1500 mg, about 650 mg to about 1450 mg, about 700 mg to about 1400 mg, about 750 mg to about 1350 mg, about 800 mg to about 1300 mg, about 850 mg to about 1250 mg, about 900 mg to about 1200 mg, about 950 mg to about 1150 mg, about 1000 mg to about 1100 mg per chewable composition.

In some embodiments, the primary ingredient is an active pharmaceutical ingredient comprising calcium carbonate, bismuth subsalicylate, dextromethorphan hydrobromide, doxylamine succinate, or any combination thereof.

In some embodiments, the chewable dosage product may include calcium carbonate, simethicone, magnesium hydroxide, magnesium citrate, docusate sodium, Dextromethorphan (DEX), doxylamine succinate (DOX), Diphenhydramine (DPH), cetirizine, loratadine, or a combination thereof.

In an example, the primary ingredient includes bismuth subsalicylate. The amount of bismuth subsalicylate in the chewable composition may range from about 3% to 10%. For example, the amount of bismuth subsalicylate may range from about 3% to about 10%, about 4% to about 9%, about 5% to about 8%, or about 6% to about 7%.

The chewable composition may have a therapeutically effective amount of an active pharmaceutical ingredient. The therapeutically effective amount may be at least 5 mg of an active pharmaceutical ingredient. In some embodiments, the therapeutically effective amount in the chewable composition may be at least 200 mg of an active pharmaceutical ingredient.

Primary ingredients may further include nutraceuticals or other dietary supplements and natural or synthetic aids for health and beauty. Primary ingredients may include nutrient supplementation including but not limited to vitamins and/or minerals for the entire spectrum of vitamins (e.g. A, B₁, B₂, B₃, B₅, B₆, B₁₂, C, D, E, K, niacin, folate or folic acid, and the like) and minerals such as iron, calcium, copper, magnesium, selenium, iodine, and zinc, as well as any other nutraceuticals and probiotics (e.g., Lactobacillus and Bifidobacterium). Further examples include but are not limited to omega-3 fatty acids, choline, antacids such as calcium carbonate or magnesium hydroxide, fiber supplements, herbal extracts such as echinacea for immune support or chamomile for calmness and sleep, or St. Johns Wort for mental wellbeing, essential amino acids or branched-chain amino acids that may support muscle health and recovery, digestive enzymes such as papain, bromelain, lactase, peptinase, and serrapeptase that may aid in food digestion, and/or other enzymes such as nattokinase may be used for a wide variety of health purposes; laxatives; sleep aids such as melatonin; antioxidants such as N-acetyl cysteine or glutathione.

Each of these compounds may be used in combination with other primary, secondary, or tertiary ingredient such as nutraceuticals or other APIs, with suitable coatings for each active when needed in order to be protected during manufacture and storage, and in order to reach their respective physiological targets by being released in the appropriate portions of the GI tract.

In referring to specific nutraceutical or pharmaceutical compounds, it is understood that the salts, chelates, and other related forms of the compound may be used, including derivatives thereof if the derivative is efficacious in a similar manner. Thus, for a chewable composition said to contain vitamin C, that vitamin may be understood to include ascorbic acid and various forms of ascorbic acid such as ascorbates (calcium or sodium ascorbate), ascorbyl palmitate, ascorbyl phosphates and salts (such as sodium, potassium, or magnesium ascorbyl phosphate), dehydroascorbic acid and salts, ascorbyl palmitate compounds, tetrahexyldecyl ascorbate, ascorbyl sulfates and salts, acylated ascorbic acid derivatives (such as 6-O-acyl-2-O-alpha-D-glucopyranosyl-L-ascorbic acids), 6-bromo-6-deoxy-L-ascorbic acid, and the like.

Primary ingredients may further include food ingredients that benefit from encapsulation to improve palatability, suppress undesirable taste attributes, reduce harsh chemical sensations, or otherwise enhance consumer acceptability. Food ingredients may include bitter compounds. Examples of bitter compounds include but are not limited to caffeine, green tea extract, guarana, cocoa polyphenols, ginseng extract, ashwagandha, and other botanical extracts known to produce bitterness or astringency. Examples of other food ingredients include vegetable concentrates and powders, bitter melon, cruciferous vegetables, mustard greens, fish, rhizomes such as ginger/galangal/turmeric, beetroot, cranberry, fermented foods such as kimchi, or berberine.

One or more primary ingredients present in the chewable composition may be distributed uniformly or nonuniformly, such as with a gradient in concentration with, for example, the center of the chewable composition having a higher concentration of an active ingredient than the outer portions of the chewable composition, or with a concentration gradient from top to bottom of the chewable composition. The difference in concentration across the gradient may be from about 20% to about 100% of the maximum concentration based on consideration of a concentration in unit volumes of 1% of the chewable composition.

The percent of the primary ingredient in the chewable composition may be from about 0.05% to about 35% by weight of the chewable composition. For example, the amount of the primary ingredient may be from about 0.05% to about 34%, from about 0.05% to about 30%, from about 0.05% to about 25%, from about 0.05% to about 20%, from about 0.05% to about 15%, from about 0.05% to about 10%, from about 0.05% to about 5%, from about 0.05% to about 1%, from about 0.1% to about 35%, from about 0.1% to about 33%, from about 0.1% to about 30%, from about 0.1% to about 25%, from about 0.1% to about 20%, from about 0.1% to about 15%, from about 0.1% to about 10%, from about 0.10% to about 5%, from about 0.10% to about 1%, from about 0.5% to about 35%, from about 0.5% to about 32%, from about 0.5% to about 30%, from about 0.5% to about 25%, from about 0.5% to about 20%, from about 0.5% to about 15%, from about 0.5% to about 10%, from about 0.5% to about 5%, from about 0.5% to about 1%, from about 1% to about 35%, from about 1% to about 30%, from about 1% to about 25%, from about 1% to about 20%, from about 1% to about 10%, from about 1% to about 5%, from about 1% to about 5%, from about 1% to about 2%, from about 2% to about 35%, from about 2% to about 30%, from about 2% to about 28%, from about 2% to about 25%, from about 2% to about 20%, from about 2% to about 15%, from about 2% to about 10%, from about 2% to about 5%, from about 2% to about 4%, from about 3% to about 35%, from about 3% to about 30%, from about 3% to about 25%, from about 3% to about 20%, from about 3% to about 15%, from about 3% to about 10%, from about 3% to about 5%, from about 5% to about 35%, from about 5% to about 30%, from about 5% to about 25%, from about 5% to about 20%, from about 5% to about 15%, from about 5% to about 10%, from about 5% to about 8%, from about 8% to about 25%, from about 8% to about 20%, from about 8% to about 18%, from about 8% to about 15%, from about 8% to about 10%, from about 10% to about 35%, from about 10% to about 30%, from about 10% to about 25%, from about 10% to about 20%, from about 10% to about 15%, from about 12% to about 35%, from about 12% to about 30%, from about 12% to about 25%, from about 12% to about 20%, from about 12% to about 15%, from about 15% to about 35%, from about 15% to about 30%, from about 15% to about 25%, from about 15% to about 20%, from about 20% to about 35%, from about 20% to about 30%, from about 25% to about 35%, from about 25% to about 30%, or from about 30% to about 35% by weight of the chewable composition. As another example, the primary ingredient may be present in the composition in an amount of about 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or about 35%.

The chewable composition further includes water. The amount of water in the chewable composition may range from about 5% to 35% by weight of the chewable composition. For example, the amount of water may range from about 6% to about 29%, about 7% to about 28%, about 8% to about 27%, about 9% to about 26%, about 10% to about 25%, about 11% to about 24%, about 12% to about 23%, about 13% to about 22%, about 14% to about 21%, about 15% to about 20%, about 16% to about 19%, or about 17% to about 18% by weight of the chewable composition. As another example, the amount of water in the chewable composition may be about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or about 35%. In some embodiments, the amount of water in the chewable composition may be about 12% to about 35% by weight of the chewable composition.

The chewable composition may further comprise insoluble solids. The insoluble solids may form at least part of the grained particles in the chewable composition or may contribute to the formation of the grained particles in the chewable composition, but are not required to form the grained particles. Non-limiting examples of insoluble solids include magnesium hydroxide and calcium carbonate. In some embodiments, the insoluble solids may also be the primary ingredient of the chewable composition.

The percent of the insoluble solids in the chewable composition may be from about 0.05% to about 35% by weight of the chewable composition. For example, the amount of the insoluble solids may be from about 0.05% to about 34%, from about 0.05% to about 30%, from about 0.05% to about 25%, from about 0.05% to about 20%, from about 0.05% to about 15%, from about 0.05% to about 10%, from about 0.05% to about 5%, from about 0.05% to about 1%, from about 0.1% to about 35%, from about 0.1% to about 33%, from about 0.1% to about 30%, from about 0.1% to about 25%, from about 0.1% to about 20%, from about 0.1% to about 15%, from about 0.1% to about 10%, from about 0.1% to about 5%, from about 0.1% to about 1%, from about 0.5% to about 35%, from about 0.5% to about 32%, from about 0.5% to about 30%, from about 0.5% to about 25%, from about 0.5% to about 20%, from about 0.5% to about 15%, from about 0.5% to about 10%, from about 0.5% to about 5%, from about 0.5% to about 1%, from about 1% to about 35%, from about 1% to about 30%, from about 1% to about 25%, from about 1% to about 20%, from about 1% to about 10%, from about 1% to about 5%, from about 1% to about 5%, from about 1% to about 2%, from about 2% to about 35%, from about 2% to about 30%, from about 2% to about 28%, from about 2% to about 25%, from about 2% to about 20%, from about 2% to about 15%, from about 2% to about 10%, from about 2% to about 5%, from about 2% to about 4%, from about 3% to about 35%, from about 3% to about 30%, from about 3% to about 25%, from about 3% to about 20%, from about 3% to about 15%, from about 3% to about 10%, from about 3% to about 5%, from about 5% to about 35%, from about 5% to about 30%, from about 5% to about 25%, from about 5% to about 20%, from about 5% to about 15%, from about 5% to about 10%, from about 5% to about 8%, from about 8% to about 25%, from about 8% to about 20%, from about 8% to about 18%, from about 8% to about 15%, from about 8% to about 10%, from about 10% to about 35%, from about 10% to about 30%, from about 10% to about 25%, from about 10% to about 20%, from about 10% to about 15%, from about 12% to about 35%, from about 12% to about 30%, from about 12% to about 25%, from about 12% to about 20%, from about 12% to about 15%, from about 15% to about 35%, from about 15% to about 30%, from about 15% to about 25%, from about 15% to about 20%, from about 20% to about 35%, from about 20% to about 30%, from about 25% to about 35%, from about 25% to about 30%, or from about 30% to about 35% by weight of the chewable composition. In an example, the insoluble solids include calcium carbonate. The amount of calcium carbonate in the chewable composition may range from about 0% to 25%. For example, the amount of calcium carbonate may range from about 0% to about 25%, about 5% to about 20%, or about 10% to about 15%.

The chewable composition may include a bulk matrix having a pH equal to or greater than 5. The bulk matrix may include one or more acid-sensitive active ingredients, which may be encapsulated with an acid-sensitive coating. The one or more acid-sensitive ingredients may be an acid-sensitive complex blended into or in contact with the chewable composition matrix.

The chewable composition may be adapted to properly deliver the primary ingredient to the human body. The chewable composition may be adapted to store and deliver the acid-sensitive active ingredient to the body of a user after ingestion.

In certain embodiments, the soft chewable composition may have a distinct melt-in-mouth texture, described herein as a “Fast Melt” texture, characterized by a very soft, pliable structure that begins to break down immediately upon mastication. The matrix may produce a gel-like, marshmallow-type consistency that may require minimal chewing and rapidly transitions from a lightly cohesive chew into a smooth, dissolving mass. The composition may maintain sufficient structural integrity for handling yet softens quickly in the oral cavity, providing a creamy, slightly chewy, and non-gritty mouthfeel. In some embodiments, the texture may be described as “melty,” “light,” “fluffy,” “smooth,” or “jelly-like,” with no chalky sensation and substantially no adhesion to the teeth. The chewable composition may dissolve quickly, leaving little to no residue or lingering aftertaste, and produces a short residence time in the mouth relative to conventional chewable antacids. This unique texture profile may allow the composition to be comfortably consumed by individuals with tooth sensitivity, by users who prefer non-chalky formats, and by populations that benefit from easy-to-chew, low-effort dosage forms.

In some embodiments, the chewable composition matrix may include emulsifiers such as lecithin, monoglycerides and diglycerides, oleates, Acacia gum, saponins, sucrose fatty acid esters, polysorbates, sodium lauryl sulfate, potassium laurate, bentonite, magnesium hydroxide, or glycerin fatty acid ester; surfactants such as wetting agents (e.g. sorbitan monolaurate), antifoaming agents (e.g. cetostearyl alcohol, lemon oil or other citrus oils, castor oil and other oils, or sorbitan trioleate); detergents (e.g. sucrose stearate); solubilizing agents (e.g. polysorbates such as polysorbate 80, propylene glycol, povidone or polyvinylpyrrolidone, cyclodextrins, glycerin or glycerol, polyethylene glycols or PEGs, and some alcohols such as ethanol, or polyethylene glycol 400 monostearate); buffering agents (e.g., phosphates, citrates, or lactates); stabilizing agents such as antioxidants (e.g., ascorbic acid, N-acetyl cysteine, propionic acid, sodium bisulfite, or sodium sulfite), chelating agents (e.g., tartaric acid, phytic acid, EDTA, malic acid, ascorbic acid, citric acid, amino acids, fumaric acid, or sodium edetate); glidants (e.g., silicon dioxide, stearic acid, magnesium stearate, calcium stearate, starches such as tapioca, pea or corn starch); colorants; sweeteners; processing aids such as vegetable oils and other lubricating agents, including stearic acid, glyceryl behenate, calcium stearate, magnesium stearate, zinc stearate, mineral and vegetable oils, benzoic acid, or polyethylene glycol; thickeners and suspending agents (e.g. alginates, xanthan gum, guar gum, carboxymethylcellulose, methylcellulose, ethyl cellulose, or Psyllium); synthetic polymers (e.g. carbomers or polyvinyl pyrrolidone), clays (e.g. bentonite, montmorillonite, magnesium aluminum silicate, or hectorite); preservatives (e.g., benzoic acid, benzalkonium chloride, benzethonium chloride, sodium benzoate, or potassium sorbate and sorbic acid), opaquing agents (e.g., titanium dioxide); perfuming agents (e.g., essential oils), glazing agents (e.g., cocoa wax, vegetable oil, beeswax, or carnauba wax), and flavoring agents.

In one aspect, an improved elevated-pH chewable composition may have a chewable composition matrix with one or more of carrageenan, agar, or derivatives thereof, wherein at least 50% by weight of the hydrocolloid contains one or more of the group consisting of carrageenan and agar, and the hydrocolloid may have less than 20% pectin and less than 20% gelatin.

Other matrix compositions may include variants of the following: (1) chewable compositions containing carrageenan; sweetening agents such as sucrose, erythritol, invert syrup and glycerol; a polyvalent metal salt, such as calcium chloride; an edible acid or salt, such as potassium citrate or sodium triphosphate, and about 5% to 15% water; (2) from 5% to 30% water, from 5% to 15% of a hydrocolloid such as kappa carrageenan dispersed in the presence of water having 0.05 M or greater of potassium or calcium ions, agar, and/or gelatin, 10% to 55% sweetener, and 2% to 20% of active ingredients selected from plant extracts, fiber, vitamins, minerals, and/or active pharmaceutical ingredients (APIs).

The composition of the chewable composition matrix will generally differ from the recipe used to create the aqueous matrix solution due to changes during manufacturing, particularly due to the loss of water as significant amounts of water may be removed during heating. For example, a gelatin-based matrix may be prepared by heating an aqueous solution of gelatin and glycerin for a time sufficient to remove from about 10% to about 80% of the initial moisture content of the aqueous solution.

In some embodiments, the chewable dosage product includes water, corn syrup, sucrose, carrageenan, bismuth subsalicylate, and calcium carbonate. In some embodiments, the chewable dosage product includes water, corn syrup, sucrose, carrageenan, bismuth subsalicylate, calcium carbonate, and optionally one or more of coconut oil, lecithin, flavors, and/or colors. The amount of water may range from about 10% to about 20% by weight of the chewable composition. For example, the amount of water may range from about 11% to about 19%, about 12% to about 18%, about 13% to about 17%, or about 14% to about 16% by weight of the chewable composition. The amount of corn syrup may range from about 15% to about 50% by weight of the chewable composition. For example, the amount of corn syrup may range from about 20% to about 45%, about 25% to about 40%, about 30% to about 35%, or about 32% to about 34% by weight of the chewable composition. The amount of sucrose may range from about 15% to about 50% by weight of the chewable composition. For example, the amount of sucrose may range from about 20% to about 45%, about 25% to about 40%, about 30% to about 35%, or about 32% to about 34% by weight of the chewable composition. The amount of carrageenan may range from about 0.5% to about 2.0% by weight of the chewable composition. For example, the amount of carrageenan may range from about 0.6% to about 1.9%, about 0.7% to about 1.8%, about 0.8% to about 1.7%, about 0.9% to about 1.6%, about 1.0% to about 1.5%, about 1.1% to about 1.4%, or about 1.2% to about 1.3% by weight of the chewable composition. The amount of bismuth subsalicylate may range from about 3% to about 10%. For example, the amount of bismuth subsalicylate may range from about 4% to about 9%, about 5% to about 8%, or about 6% to about 7% by weight of the chewable composition. The amount of calcium carbonate may range from about 2% to about 25% by weight of the chewable composition. For example, the amount of calcium carbonate may range from about 5% to about 20% by weight of the chewable composition, about 10% to about 15%, or about 12% to about 14% by weight of the chewable composition. The amount of coconut oil may range from about 0% to about 5% by weight of the chewable composition. For example, the amount of coconut oil may range from about 0.1% to about 4.5%, about 0.5% to about 4.0%, about 1% to about 3.5%, about 1.5% to about 3.0%, or about 2.0% to about 2.5% by weight of the chewable composition. The amount of lecithin may range from about 0% to about 2% by weight of the chewable composition. For example, the amount of lecithin may range from about 0.1% to about 1.75%, about 0.5% to about 1.5%, about 0.75% to about 1.25%, about 1.0% to about 1.2%, or about 1.1% to about 1.15%. The amount of flavors may range from about 0% to about 2%. For example, the amount of flavors may range from about 0.1% to about 1.75%, about 0.5% to about 1.5%, about 0.75% to about 1.25%, about 1.0% to about 1.2%, or about 1.1% to about 1.15%. The amount of colorants may range from about 0% to about 1% by weight of the chewable composition. For example, the amount of colorants may range from about 0.1% to about 0.9%, about 0.2% to about 0.8%, about 0.3% to about 0.7%, about 0.4% to about 0.6%, or about 0.5% to about 0.55% by weight of the chewable composition.

In another aspect, an improved chewable composition is described in which a high-pH chewable composition matrix is provided that is adapted to be compatible with an acid-sensitive active ingredient, an active ingredient with an acid-sensitive coating, or an acid-sensitive complex, such that they are not damaged by contact with the chewable composition matrix and can withstand prolonged storage without significant loss of activity or other unfavorable interactions with the ingredients of the chewable composition. Such materials may be incorporated into a chewable composition without the need for coating the active ingredients with an encapsulant to cover and protect the active ingredients. This encapsulated active may be beneficial when the desired loading of an active ingredient is relatively high such as from about 20 mg/g to about 600 mg/g of the chewable composition matrix. For example, the loading may be greater than 20 mg/g of chewable composition matrix, or more than 30 mg/g, 50 mg/g, 75 mg/g, 100 mg/g, 125 mg/g, 150 mg/g, 175 mg/g or 200 mg/g, such as from 20 to 500 mg/g, or from 50 to 600 mg/g, or when the intended loading of the active ingredient is more than 5% of the mass of the chewable composition matrix, such as more than 7%, 10%, 15%, or 20% of the mass of the chewable composition matrix, such as from 5% to 50% or from 10% to 70%. In such cases, for example, adding a protective coating or encapsulant would greatly increase the occupied volume or mass and could compromise the chewable composition in several ways.

The chewable composition is provided in which the matrix may have a pH above 5.5, such as from 5.5 to 9, 5.6 to 8.9, 5.7 to 8.8, 5.8 to 8.7, 5.9 to 8.6, 6.0 to 8.5, 6.1 to 8.4, 6.2 to 8.3, 6.3 to 8.2, 6.4 to 8.1, 6.5 to 8.0, 6.6 to 7.9, 6.5 to 7.8, 6.6 to 7.7 or 6.7 to 7.6. Such a pH level may be useful in providing certain taste benefits for selected flavorings and may be useful in combination with some nutraceuticals or other primary ingredients that may not need encapsulation for protection at that pH. But encapsulated ingredients that target the small or large intestines may have a primary coating that releases the active at a pH above 5 or above 4.5, for example, and thus would be in danger or premature release if embedded in a matrix with an elevated pH. This damage may be mitigated by adding a secondary coating that dissolves or weakens at low pH, such as a pH less than 3.5, 3.0, or 2.5, allowing the secondary coating to remain intact when exposed to the aqueous matrix solution and then to the gelled matrix, but able to disintegrate or weaken in the stomach, allowing the primary coating to be exposed there but to remain intact until it passes beyond the stomach to the desired region(s) of the GI tract after the stomach where pH is more elevated. For example, for a product intended to release melatonin after the stomach as a sleep aid, particles of melatonin may be coated in a spray dryer or fluidized bed using a primary coating designed to release the active at a pH above 4.5 (e.g., from about 5 to 6 for the small intestine, or from about 6 to 7.5 for the large intestine), such as a resistant starch, a shellac, zein, cellulose acetate phthalate (CAP), hydroxypropylmethyl cellulose phthalate (HPMCP), or polyvinyl acetate phthalate (PVAP). Such coatings may be resistant to pH levels below 4.5 but dissolve at pH levels above 5.5. In addition to that primary coating, there may be a secondary coating on top of the primary coating designed to weaken or dissolve in the low pH environment of the stomach. The secondary coating protects the primary coating and its active from the elevated pH environment of the matrix solution and the gelled matrix during storage.

The chewable composition may deliver two or more functional benefits. The two or more functional benefits include but are not limited to allergy relief, relief from constipation, relief from stomach pain or discomfort due to excess stomach acid or acid reflux, and the like.

The chewable composition of any of the preceding aspects or variants thereof may be adapted to relieve from one or more of the maladies including but not limited to allergies, constipation, stomach pain, diarrhea, flatulence, and discomfort due to excess stomach acid or acid reflux. In some embodiments, the chewable composition of any of the preceding aspects or variants thereof, the at least one acid-sensitive active ingredient, active with an acid-sensitive coating, or acid-sensitive complex may provide an allergy-relief benefit, a laxative function, an antacid function, or a combination thereof.

Direct measurement of the pH of a chewable composition matrix may be made using a pH meter with a suitable electrode adapted for measurement of semi-solids or soft solids. In particular, a conical electrode probe with a glass body refillable single-junction electrode probe with a triple ceramic junction in the outer reference cell and a conical pH sensing portion made with low temperature glass, adapted such that 40 to 50 μl/hr of electrolyte can flow. For example, the HI1612D pH electrode from Hanna Instruments may be used. The chewable composition should be conditioned for 48 hours at 50% relative humidity and 23° C. before the measurement is made, and then the electrode should be pressed against a flat portion of the chewable composition to make full contract of the conical tip with the chewable composition, and contact should be maintained for 60 seconds to obtain a reading. A second measurement is then made by cutting the chewable composition in half to expose an interior portion of the chewable composition matrix and repeating the process for another measurement after 60 seconds of full contact with the conical tip. In both measurements, longer times may be needed if the measurement is not stable (i.e., a change of more than 0.2 units of pH over 30 seconds). The two measurements are then averaged and reported as the pH of the chewable composition. Five chewable compositions are measured in this way and the average is reported as the average direct pH.

An indirect pH measurement may also be made by taking between a 5-10 gram sample of the chewable composition matrix and dissolving it in about 50 mL to about 100 mL of water at 40° C. under agitation for three minutes, decanting immediately to remove any solids, then cooling to 23° C. and measuring the resulting pH with a standard pH meter. This is done once for each of five chewable compositions and the average reading is then reported as the average indirect pH. In some aspects, the average direct measurement differs from the average indirect pH by less than 0.5 units, and in some aspects, the average direct measurement is greater than the average indirect pH by 0.3 to 0.7 units, or at least 0.2 units.

The chewable composition meets key characteristics of performance. The key characteristics of performance include but are not limited to palatability, stability, release profile, and/or the assay of the active. In some embodiments, the key characteristics of performance may be related to palatability. Palatability means the chewable composition possesses a substantially non-bitter taste. This may be achieved through the addition of flavor enhancers, bitter blockers, sweeteners, or other flavor masking agents, or through encapsulation or complexation of the active ingredient.

In another aspect, the key characteristics of performance may be related to stability. Stability means the hardness or robustness of the chewable composition to withstand the rigors of manufacturing, packaging, shipping, and distribution, as well as to be easily chewed by the intended patient population. This may include limiting syneresis to an acceptable level. It may also include the acid-sensitive active ingredient, active with an acid-sensitive coating, or acid-sensitive complex not being damaged by contact with the acid-sensitive chewable composition matrix and being able to withstand prolonged storage without significant loss of activity or other unfavorable interactions with the ingredients of the acid-sensitive chewable composition. A significant loss of activity may be defined as any loss in assay that places the final product out of spec as defined in the final project quality spec. This may also include having and maintaining textural characteristics comparable to that of a traditional low-pH chewable composition.

In another aspect, the key characteristics of performance include release profile. Release profile means the dissolution profile of the chewable composition and the ability to properly release/deliver the active in the targeted region. Target region refers to an area of the GI tract. The release profile may include one or more of extended release, delayed release, immediate release, pulsatile release, biphasic or multiphasic release, responsive release (e.g., responsive to a pH), and chronotherapeutic release.

A suitable release profile may be when no more than about 80% of the API may be released within about 6 hours in vitro as measured in a USP approved Type 2 paddle apparatus at 50 rpm or 100 rpm at a constant temperature of 37±0.5° C. A suitable release profile may be when no more than about 75% of the API may be released within about 6 hours in vitro as measured in a USP approved Type 2 paddle apparatus at 50 rpm or 100 rpm at a constant temperature of 37±0.5° C. A suitable release profile may be when no more than about 70% of the API may be released within about 6 hours in vitro as measured in a USP approved Type 2 paddle apparatus at 50 rpm or 100 rpm at a constant temperature of 37±0.5° C.

In another aspect, the key characteristics of performance include performance of the primary ingredient, particularly when the primary ingredient is an active pharmaceutical ingredient. Performance of the active pharmaceutical ingredient means the active pharmaceutical ingredient maintains intended bioavailability and efficacy as measured through either quality control tests and clinical tests including but not limited to acid neutralizing capacity, anti-foaming, dissolution, or isolated organ bath system.

In a related example, the encapsulated melatonin may be a first active and a second active may be probiotics, which may be encapsulated in a similar manner with a primary and secondary coating to withstand an alkaline matrix but to also ensure that the active ingredient with its primary coating passes through the stomach to be released in the small or large intestine. The probiotics may comprise a different primary coating that is more compatible with the probiotics, such as a cellulosic primary material or a lipids coating (e.g., ceresin wax, cocoa butter, beeswax, cetyl palmitate, or rice bran wax) prior to the acid-resistant coating, followed by an acid-sensitive coating.

### Encapsulation Methods and Materials

In some embodiments, the primary ingredient, such as an acid-sensitive active pharmaceutical ingredient, may be protected in an encapsulating complex having an internal medium into which the primary ingredient is embedded. The encapsulating complex includes the primary ingredient. In some embodiments, the internal medium may be compatible with the primary ingredient and may provide protection from one or more challenges that the primary ingredient may face when combined with a chewable composition. The encapsulating complex may be in the form of a pellet embedded within or attached onto the chewable composition matrix, or a plurality of relatively small sections of active ingredients surrounded by an internal medium, or may be provided as coating attached to a portion of the chewable composition matrix, such as a top coat, or a coating that largely encases the entire chewable composition matrix. Such a coating may be relatively thin, such as less than 2 mm, or about 1 mm or less, or may be relatively thick such as greater than 2 mm, greater than 3 mm, or greater than 4 mm, such as from 2 mm to 20 mm in thickness or from 2.5 mm to 8 mm. In some aspects, the mass of the encapsulating complex may be from about 25% to about 80% or more of the mass of the chewable composition. For example, the mass may be about 30% to about 75%, about 35% to about 70%, about 40% to about 65%, about 45% to about 60%, or about 55% to about 57% of the mass of the chewable composition.

In some aspects, the encapsulating complex contains one or more lipids such as stearic acid, myristic acid, arachidic acid, or other fatty acid or mixture of fatty acids having a fatty acid with 14 or more carbons or 16 or more carbons, or includes palm oil, palm kernel oil, coconut oil, cocoa butter, shea butter, mango seed oil, babassu oil, illipe butter, sal butter, kokum butter, milk fat, butter, cream, or hydrogenated forms of any of the listed lipids. The amount of such lipids may be at least 10%, 20%, 30%, 40%, or 50% by weight relative to the mass of the encapsulating complex or the mass of the internal medium of the encapsulating complex. In some aspects, the internal medium may comprise at least 10 wt % of chocolate, a tropical fat, or oil such as cocoa butter or coconut oil, and an animal fat such as milk fat or butter, while also containing at least 10% by weight of a sweetener such as sugar.

In some aspects, the encapsulating complex may include lipids, sweeteners, and flavorings, and in some aspects, the flavorings may include one or more chocolate flavorings such as cacao powder or liquor, carob, fruit extracts such as date preserves. In some cases, the encapsulating complex comprises chocolate. In some aspects, the active ingredients comprise probiotics that have been encapsulated for flavor masking and optionally with a release-regulating coating layer to control release to a suitable region in the GI tract such as the intestines.

In some aspects, protective lipids for an active ingredient particulate are combined by soaking the molten lipid into the pores of the active ingredient via capillary suction and/or by vacuum suction. The lipid treated particles may still be relatively free flowing and capable of further treatments such as encapsulation in a fluidized bed or spray coating operation. Lipid-treated particles may then be further encased in an internal medium that may comprise the same or other lipids, or be substantially lipid free, or the lipid-treated particles may be coated with another encapsulant such as shellac, cellulose derivatives, or other polymers to form an encapsulating complex in which lipids form a protective internal medium in contact with the active ingredient and further protected in the other encapsulant. In a related aspect, dry oil powders may be prepared in which the active ingredient may be provided in an oil-water emulsion with stabilizing materials such as proteins, coacervates, nano-cellulose or other fibrous material, followed by removal of the water to provide an oil-rich composition (e.g., over 50% oil by weight, or over 60, 70%, 80%, 90%, or 95% oil) that nevertheless may, in some aspects, be relatively free flowing and capable of further encapsulation. In some aspects, the dry oil powder containing the active ingredient may be directly added to a chewable composition matrix or may be further encapsulated, such as in an encapsulating complex such as a relatively large encapsulating complex with an effective diameter of between about 2 mm or greater or about 4 mm or greater.

The sensitive primary ingredient may comprise probiotics or other temperature-sensitive, shear-sensitive, moisture-sensitive, or acid-sensitive materials that may be rendered less effective if exposed to the environment of conventional chewable composition manufacturing processes. Individual particles or clusters of the sensitive primary ingredient may be encapsulated by any known method (coating, complexation, or inclusion in a hot melt) to provide a release-regulating coating layer for delayed or extended release, or to otherwise help ensure that the primary ingredient is properly released in the body; alternatively, encapsulation of particles or clusters may be provided for other purposes such as preventing or mitigating release of unpalatable flavors or unwanted aromas into the chewable composition matrix that would be unpleasant during or prior to ingestion. The encapsulating complex may be formed as a macroscopic pellet of material in a 3D shape such as a sphere, ellipsoid, cylinder, pyramid, cube, parallelepiped, torus, disk, rectangular prism, cone, tetrahedron, octahedron, dodecahedron, icosahedron, capsule, ovoid, hemisphere, frustum, or more complex shapes such as 3D stars, or flowers.

An improved approach to encapsulation to address one or more of the gaps in current chewable compositions and manufacturing employs the use of an encapsulating complex embedded in or attached to a chewable composition, wherein the encapsulating complex comprises the primary ingredient embedded within an internal medium. The internal medium may then be further encapsulated with an encapsulant on the surface of the internal medium if desired. For example, in some cases, the internal medium may be a meltable material such as a lipid that prevents moisture from damaging the primary ingredient therein, and the internal medium may further have a shell of shellac or other durable material that may protect the internal medium from high temperatures that would otherwise cause the internal medium to melt and disperse, but with the restraint of a more thermally stable shell or outer coating, the internal medium and the primary ingredient can maintain integrity and stay in place even when the internal medium is temporarily softened or melted. The internal medium and its optional shell may also provide a degree of thermal insulation to protect the primary ingredients from elevated temperatures during manufacturing.

In general, the encapsulating complex may further include one or more coatings to further protect the internal medium and the primary ingredient from contact with a heated aqueous matrix solution used to form the matrix of a chewable composition, or from contact with the matrix of the chewable composition after cooling has begun, or to reduce diffusion or migration of materials into or out of the internal medium, such as water into the primary ingredient or flavors or odors from the primary ingredient to the matrix of the chewable composition.

In some aspects, the encapsulating complex is intended to break up during chewing of the chewable composition, releasing the internal medium in the environment of the mouth. Thus, the internal medium may have a flavor, texture, and mouthfeel that can contribute to a positive user experience during ingestion (e.g., the internal medium may include chocolate or chocolate-like components, a flavored gelatin or pectin matrix, or a flavored syrup), and may include characteristics that are distinctly different than the flavor and mouthfeel of the matrix that encases or is attached to the encapsulating complex. For example, the matrix of the chewable composition may have a mint flavor, and the internal medium of the encapsulating complex may have a chocolate cream that, when released, yields a pleasant chocolate mint combination, while still allowing a flavor-masking coating on an encapsulated primary ingredient in the encapsulating complex to remain intact and prevent exposure to a bitter or unpalatable flavor of the primary ingredient.

In some aspects, the primary ingredient comprises sensitive material such as bacterial probiotics that might be rendered inactive if they were placed directly into the hot aqueous matrix solution for the chewable composition during manufacturing, with temperature, shear, moisture level, and pH being among the possible challenges that could jeopardize the activity or function of the primary ingredient. An approach to protect sensitive materials has been developed in which the primary ingredient is first embedded in the internal medium of an encapsulating complex, which in turn may be coated with a resistant materials such as a shellac or other polymer, and wherein the encapsulating complex is not blended into the aqueous matrix solution prior as materials are being blended and heated to form a hot aqueous matrix solution, but is only contacted with the aqueous matrix solution after its properties have changed substantially from the peak levels of the challenge or challenges of concern. Thus, an encapsulating complex having temperature-sensitive materials may be combined with the aqueous matrix solution only after the aqueous matrix solution has begun cooling from its peak temperature. Combining the encapsulating complex into the hot aqueous matrix solution thus may be done when the aqueous matrix solution is at a temperature substantially lower than the peak temperature achieved during cooking of the aqueous matrix solution, such as at least 5° C., 10° C., 15° C., 20° C., 25° C., 30° C., 35° C., 40° C., or 45° C. less than the peak temperature, such as from 5° C. to 65° C. less than peak temperature, 10° C. to 50° C. less than peak temperature. Peak temperature may, for example, be at a boiling point of the aqueous matrix solution after a substantial amount of the initial moisture has evaporated, such as at least 10%, 20%, 30%, 40%, 50%, or 60% of the initial moisture, with a peak temperature of at least 90° C., 95° C., 100° C., 105° C., 110° C., 115° C., 120° C., and 125° C., such as from 110° C. to 130° C. or from 90° C. to 120° C.

In some aspects, the encapsulating complex is inserted into a matrix solution in a mold by injection or other means, and may be inserted in a manner that displaces part of the matrix solution or may be inserted into a void in the matrix, a depression on the matrix, or attached to surface of the matrix. In some aspects, a mold may be partially filled or completely with an aqueous matrix solution or slurry, followed by addition of one or more encapsulating complexes.

One or more primary ingredients within the encapsulating complex may be provided with coatings or barrier materials independent of the internal medium and any coating it may have. Such materials may be release-regulating coating layers designed to control the timing of release of the primary ingredient once ingested (e.g., immediate release or extended release), or maybe flavor-masking materials. Thus, in some aspects, one or more tailored materials are applied to microencapsulate one or more primary ingredients to mask unpleasant flavors or aromas, and may also protect the one or more primary ingredients from challenges faced during manufacturing and storage, while also providing, supporting, or being compatible with the intended release profile for the one or more primary ingredients.

The release-regulating coating layer may be adapted to govern the release profile after ingestion without substantial interference from the flavor masking coating layer or other protective layers.

The encapsulating complex or any component therein may be provided with an additional protective layer to prevent premature opening of the encapsulating complex or to protect the encapsulating complex from harmful interaction with matrix of the chewable composition such as adverse interactions with the moisture, pH, or other chemical agents in the matrix or in other encapsulated materials therein, or to mitigate oxidation or other challenges from the environment during manufacturing and storage. Thus, in some aspects, a chewable composition is described having a matrix and at least one encapsulating complex embedded within or attached to the matrix, wherein the encapsulating complex comprises at least one primary ingredient disposed within an internal medium adapted to protect the at least one primary ingredient from one or more challenges such as pH, oxidation, moisture, elevated temperature, and wherein the internal medium is in turn provided with an outer coating or shell to further protect the internal medium from one or more challenges such as elevated temperature.

The release-regulating coating layer may be provided as the first layer applied to a primary ingredient, such that the materials of the release-regulating coating layer are in contact with the primary ingredient or are at least closer to the primary ingredient than a flavor masking coating layer. In such aspects, the release-regulating coating layer could be the primary coating layer on the primary ingredient with a further outer secondary coating layer, the flavor masking coating layer, which is applied to provide added masking efficacy beyond whatever masking may be provided by the release-regulating coating layer.

While in some aspects, multiple layers may be concentric, with clear boundaries between the materials of each layer, in other embodiments, the materials may be interdigitated or interspersed for at least part of the coating thickness. This interdigitation or interspersed state need not be construed as miscibility, but generally rather as a physical intermingling of the distinct materials. Such layers may be applied at substantially the same time, as in a spray drying system or fluidized bed system with nozzle delivering droplets of both coating materials simultaneously, or with alternating bursts of differing materials being applied, or in multiple cycles as one coating material at a time is applied to build a lamellar or other complex structure.

In a related aspect, the protective function for each of the one or more coatings may be protecting its respective primary ingredient or any internal coating layers from at least one of the group consisting of: (1) a maximum exposed temperature Tₘₐₓ encountered during blending of the one or more one primary ingredients with the aqueous phase, (2) a maximum shear level τₘₐₓ experienced during blending, (3) the moisture level of the aqueous phase or maximum activity level aₘₐₓ of the water in the aqueous phase during manufacturing and/or during shelf life (storage after manufacturing), (4) adverse reactions with other ingredients in the chewable composition, (5) oxidation, and (6) unwanted damage to (e.g., etching, erosion, fracture, or dissolution of) a coating layer intended to control release of the primary ingredient in the GI tract. Further, any one or more of the coatings may also provide a masking function relative to the odor or flavor of a primary ingredient.

In some aspects, two or more types of encapsulating complex may be present with distinct coating formulations, such as two or more types of outer coatings, that provide two or more different protective functions, or different degrees of protection relative to the various challenges faced during manufacturing and storage. For example, a first encapsulating complex may have an outer coating designed to resist elevated shear (e.g., to protect internal materials that are fragile and might break or be released prematurely when subject to the shear stresses of mixing) more effectively than the coatings on other encapsulating complexes, while a second encapsulating complex may have a coating designed to resist elevated moisture (e.g., to protect a moisture-sensitive coating on an primary ingredient embedded in the internal medium) for extended release once in the stomach or intestines.

In one aspect, a chewable composition is described having a first primary ingredient with a primary coating such as an enteric coating designed to reach a targeted region of the GI tract in order to deliver the primary ingredient at a suitable region therein (e.g., probiotics to the small intestines and colon, iron to the stomach, a bitter agent coated in order to mask the flavor while in the mouth), while also being coated with a suitable secondary coating to improve the stability of the first primary ingredient, and further being embedded in an internal medium which may be a meltable material such as a lipid or polymer that is solid or semi-solid at 25° C. but has a melting point from 28° C. to 80° C., 30° C. to 70° C., 35° C. to 65°, 40° C. to 60° C., or 45° C. to 55° C. and may be further protected with a shell or coating that is stable at higher temperatures such at least about 5° C., about 10° C., or about 15° C. above the melting point of the internal medium.

In one aspect, one or more coating layers on an encapsulated primary ingredient in or on the matrix of a chewable composition is designed to prevent migration of water into the primary ingredient, while also limiting the migration of the primary ingredient into the water of the matrix or into the matrix itself. Maintaining the primary ingredient in a relatively dry state segregated from the matrix during storage and from the aqueous solution used to create the matrix during manufacturing may be achieved to various degrees with the methods, processes, and compositions described herein. Such “waterproofing,” though believed to be rarely considered in prior work, has been found in our investigations to be a useful consideration. Thus, in some aspects, a water-resistant coating is provided as a layer of an encapsulated primary ingredient, wherein the water-resistant coating is adapted to withstand the moisture level or water activity of the aqueous solution during manufacturing and also to withstand the moisture level or water activity of the resulting matrix after manufacturing and during storage over the shelf life of the chewable composition.

In one aspect, stability of a primary ingredient in or on a chewable composition matrix is enhanced by coating the active ingredient with a moisture resistant material that prevents direct contact with the aqueous matrix solution during manufacturing and resists migration of water into the active ingredient during storage while the active ingredient is in or on the matrix of a chewable composition. Such water-resistant materials may include shellac, such as shellac that is also coated with or combined with a hydrophobic material such as a wax, a polyolefin such as polyethylene or polypropylene. Also to be considered are acrylate polymers (e.g., poly-ethylacrylate, poly-methylmethacrylate), Eudragit coatings, ethylcellulose, polyolefins, zein, waxes and fatty acids, latexes such as those made from ethylcellulose and related polymers, combinations of nanocellulose with any of the polymers mentioned herein, including coated nanocellulose, methacrylate aminoester copolymers, or methacrylate ester copolymers. Such coatings may also be associated with, contain, or be combined with oxygen scavengers, antioxidants, swellable materials such as hydrogels that can swell in response to high moisture content to preclude further migration of moisture through the hydrogel, further hydrophobic agents, or crosslinkers.

Various polymers may be selected to construct a coating for encapsulation of ingredients. For coatings adapted to provide masking effect relative to a flavor or odor that may be unpleasant, compounds that may be considered include polyacrylate-based polymers such as Eudragit materials. For example, Eudragit E is a cationic polymer based on dimethylaminoethyl methacrylate, butyl methacrylate, and methyl methacrylate. It is soluble in gastric fluid as well as in weakly acidic buffer solutions, up to pH~5. When serving as an outer layer in microencapsulation, it forms swellable, permeable, and insoluble films at pH 5 or higher (as in saliva, with a pH from about 6.7-7.6, yet dissolves rapidly by forming salts at a pH less than 5 such as in gastric fluids with a pH from about 1.0 to 1.7.

Hydroxypropyl methylcellulose (HPMC) may also be used and can form a gel-like matrix that can help control the release of the primary ingredient in the GI tract while also reducing the risk of unpleasant flavors or odors. Polyvinyl alcohol (PVA) may be used for its film forming capabilities that can control the release of the primary ingredient while also being able to plasticize several film-forming encapsulants. Alginate such as sodium alginate or calcium alginate is a natural polymer with potential to control the release of some primary ingredients and help mask flavors. Gelatin can also be used in masking flavor and can also aid in controlled release of a primary ingredient, as can chitosan, a natural polymer derived from chitin. Film-forming salts of chitosan (e.g., chloride, lactate or gluconate) may also be considered for flavor and aroma masking.

Such coatings may be applied in one or multiple layers, and may include lipids such as glycerides and waxes that may have a melting point or melting range of at least 20° C. such as from 20° C. to 150° C., from 30° C. to 150° C., from 40° C. to 150° C., from 30° C. to 110° C., or from 35° C. to 90° C., or, in some aspects, may have a melting point or range such that the coating will soften in the mouth. Such waxes, for example, may include synthetic and natural waxes such as animal waxes, vegetable waxes, or petroleum waxes, including waxes such as beeswax, lanolin, bayberry, candelilla, sunflower, carnauba, paraffin, microcrystalline petrolatum and carbowax alone or in mixtures. Alternatively or in addition, the coatings may include various polymers such as proteins, polypetptides or poly-amino acids such as polyglutamic acid, polyaspartic acid, and polylysine; polyacrylamide; poly N-acylhydroxypropine esters; polylactic acid; polyglycolic acid; polylactic-co-glycolic acid; polysebacic acid; polyfumaric acid; polymers formed from hydroxyethylmethacrylate; polymers formed from ethylene bismethacrylate; carboxymethylcellulose and its derivatives; gum arabic; agarose; alginate such as sodium or potassium alginate; polyphosphate; heparin; gelatin; guaran; copolymers of sebacic acid and fumaric acid; copolymers of biscarboxyphenoxypropane and sebacic acid; poly(carboxyphenoxyacetic) acid; poly(carboxyphenoxyvaleric) acid; poly-ε-caprolactone and related polyesters; poly-ε-caprolactone-co-δ-valerolactone; poly-ε-caprolactone-co-lactic acid; hyaluronic acid; chitin; chitosan; dextran; dextrin, carboxy-dextran, diethylaminoethyl dextran, aminoethyl dextran and dextran sulfate; collagen; albumin; fibrinogen; and other naturally occurring polymers, such as collagen, starch, amylose, carboxymethyl amylose, hydroxyethyl amylose, hydroxyethyl amylose, and cellulose, carboxylmethyl cellulose, hydroxyethyl cellulose; agarose, pectic acid, alginic acid, gum arabic, galactomannan, levan, hyaluronic acid; polyalkylene glycols such as polyethylene glycol, polypropylene glycol, polybutylene glycol and mixtures thereof.

Synthetic and natural waxes useful as coatings for the ingredients include animal waxes, vegetable waxes, petroleum waxes and the like. Specific examples of waxes are beeswax, lanolin, bayberry, candelilla, carnauba, paraffin, microcrystalline petrolatum and carbowax. Mixtures of these waxes are also useful. and so forth, as well as derivatives or mixtures thereof.

Encapsulants may also include nanocarriers, including nano-delivery systems that carry drugs on the nanoscale and may provide improved drug stability, controlled release, or targeted delivery. Such nanocarriers may include: (1) Vesicular systems that involve vesicles, or small, enclosed sacs that can carry drugs, which may include liposomes, niosomes, and catanionic vesicles, for example, including phospholipid systems such as those containing lecithin or other phospholipids that may help form vesicles; (2) Colloidal drug delivery systems, such are mixtures in which one substance is dispersed throughout another at a colloidal scale, which may include liposomes, niosomes, or micelles; (3) Bilayer systems in which a bilayer of lipids or surfactants that form a sphere that can enclose a primary ingredient; (4) Self-assembled structures such as micelles and vesicles, that are sometimes referred to as self-assembled or self-organizing systems; (5) Surfactant-based systems, which refers to systems that utilize surfactants to improve drug delivery. These may include micelles, niosomes, and catanionic vesicles; or (6) Lipid-based delivery systems may, for example, include liposomes and solid lipid nanoparticles and nanostructured lipid carriers. Liposomes may be created from cholesterol and natural non-toxic phospholipids to encapsulate both hydrophilic and lipophilic drugs, protect the drug from degradation, and control the release of the drug. Depending on their design, liposomes can potentially deliver a drug to various parts of the gastrointestinal (GI) tract, not just the stomach. Niosomes likewise are vesicles employing non-ionic surfactant vesicles that, like liposomes, can encapsulate both hydrophilic and lipophilic drugs. Related structures employing cationic and anionic surfactants may be considered, including catanionic vesicles. The surface charge of cationic vesicles can also be tuned by adjusting the ratio of cationic to anionic surfactant, which can influence the vesicles' interaction with biological membranes and their release behavior.

Micelles, which are formed from surfactants in solution, can also be used for drug delivery. Unlike vesicles, which have an aqueous core surrounded by a lipid bilayer, micelles have a hydrophobic core surrounded by a hydrophilic shell. This makes them particularly suitable for the encapsulation and delivery of lipophilic drugs.

By way of example, encapsulation may be achieved by coating a primary ingredient with coating agents. Coating agents include but are not limited to polysaccharides or polymers, including chitosan, sodium alginate and other alginate salts, xanthan, curdlan, guar gum and other polysaccharides (particularly ionically crosslinkable polysaccharides), as well as Eudragit (polymethylmethacrylate polymers), as encapsulating agents. Polysaccharides or polymers may be used as coating agents that may be sprayed on to ingredients to create a barrier between the primary ingredient and its environment.

Hydrogel coatings on ingredients may be used to enhance control of the release of ingredients and enhance the stability of the ingredients. For example, hydrogels can swell when in contact with an aqueous environment and thereby modify the diffusion paths for the primary ingredient, and stimuli-responsive hydrogels in particular such as poly(methacrylic acid) nanogels can swell in response to a change in conditions such as a rise in pH, which can make them useful in controlled release applications. Initiated chemical vapor deposition may be used to form a film that can encase ingredients in the hydrogel coating without the use of a solvent that might threaten the stability of the primary ingredient. Such material may be adapted to prevent diffusion of a primary ingredient when in a low pH environment such as chewable composition as described herein, but when the pH increases such as in the GI tract after the stomach, the hydrogel can change to allow greatly increased diffusion. A crosslinker may be varied in concentration in order to adjust the hydrogel performance.

In some aspects, a hydrogel material may be used for components of the chewable composition such as an encapsulating complex encasing one or more ingredients with a secondary matrix within the complex surrounding the primary ingredient. Primary ingredients may be encapsulated with or otherwise combined with one or more hydrogels via a wide variety of methods, including spray drying and vapor-phase deposition methods such as initiated chemical vapor deposition (iCVD), plasma enhanced chemical vapor deposition (PE-CVD), and atomic layer deposition (ALD).

Chitosan, alginates such as sodium alginate, xanthan, curdlan, guar gum and other polysaccharides (particularly ionically crosslinkable polysaccharides) may be considered as hydrocolloids. Cellulose acetate phthalate (CAP) and other cellulose derivatives may be considered, as well as cellulose acetate trimellitate (CAT), polyvinyl acetate phthalate (PVAP), polyvinylacetate phthalate, HPMCP, poloyethyene glycols, and the like.

By way of example, starch and other polysaccharides may be considered as gelling agents for thickening. Starches may be fabricated into porous starch, microgels, molecular aggregates, starch granule aggregates, and other forms of various porosity, diffusivity, solubility, and enzymatic resistance, properties that may be adapted to enhance the stability of one or more primary ingredients. Such systems may be used to encapsulate, protect, and deliver many ingredients or other ingredients. Starch-based coatings may be applied by spray drying, extrusion, freeze drying, sonication using ultrasound or ultrasonic homogenization, ultra-high pressure, coagulation, fluidized bed coating, molecular inclusion, chemical, and enzymic methods. For example, a starch combined with oleic acid or other lipids may be combined and coated onto a particle to provide a delayed release of controlled release mechanism. In general, controlled release of ingredients may be achieved with starch-based microcapsules (or nanocapsules) that encapsulate the ingredients. Such encapsulants can help provide bioavailability, resistance to enzymes, protection of probiotics, masking of bitterness or other bad flavors, and may be tailored to reach the small intestines, colon, or stomach. A variety of starch-based microcapsule wall materials, may be considered, such as cyclodextrins and related crown compounds, amylose, octenylsuccinic anhydride (OSA)-modified starch, and numerous other modified starches such as cationic starch, anionic starch, oxidized starch, cross-linked starch, acid-hydrolyzed starch, amylolytic starch, or debranching starch. Starches combined with lipids may be considered for a variety of water-resistant layers.

Starch-based microcapsule delivery systems often employ starch that has been modified by physical, chemical or enzymatic methods. Such physical methods may include (1) microwave treatment which may be carried out with the starch in the presence of lipids such as linoleic acid, oleic acid, stearic acid, ethyl cis-9-octadecenoate, or ethyl cis,cis-9,12-octadecadienoate and methyl octadecenoate; (2) high pressure homogenization, wherein high shear, impact and cavitation can break down starch granules as high-pressure liquid passes the gaps of a homogenizer valve, resulting in dissolving of amylose that can then be compounded with other molecules to form a complex; (3) ultrasonic treatment, in which the cavitation produced by ultrasonic waves in water can result in high shear that can break down starch granules and assist in preparing a starch-based encapsulant; (4) ultra-high pressure treatment that can modify hydrogen bonding in starch and lead to improved combinations with other materials resulting in hydrogels; (5) high-speed shearing; and/or (6) freeze drying.

Chemical methods for modifying starch may include (1) esterification that may, for example, be used to provide OSA-modified starch, which may further be combined with gum arabic or other agents to yield a useful encapsulant mixture; (2) oxidation; (3) acid hydrolysis; and (4) cross-linking (e.g., sodium trimetaphosphate/sodiumtripolyphosphate cross-linking, which may be done, for example at elevated pH such as 10 or higher or 11 or higher in the presence of sodium sulfate).

Enzymatic methods may include production of amylolytic starch using enzymes such as α-amylase, β-amylase, and glucosidase, which may be useful in producing porous starch. Debranching starch can also be produced, such as through treatment of starch usually treated with starch debranching enzymes such as pullulanase and isoamylase. Cyclodextrin containing 6-12 D-glucopyranose units may also be considered a type of enzymatically modified starch since it is a cyclic oligosaccharide that may be produced from amylose in the presence of cyclodextrin glucosyltransferase found in Bacillus bacteria.

Modified starches can also include cationic and anionic starches, whose charge characteristics may be helpful in encapsulating specific compounds. Also to be considered is resistant starch. Resistant starch generally can resist the hydrolysis from the enzymes in the stomach and the duodenum and can then be degraded by microbiota in the colon. Resistant starch has four broad classes, Types 1, 2, 3, and 4. Type 1 may be naturally occurring starches that resist digestion, including those found in legumes and milled grains. Type 2 has non-gelatinized granules that may be slowly digested in the small intestine but largely reach the large intestine and are found in green bananas, raw potatoes, or high amylose corn. Type 3 is cooked in water until fully hydrated and is then cooled. Type 4 includes chemically modified starches that may be difficult to digest.

For delivery of a primary ingredient after the stomach, various enteric coatings may be considered that can withstand the low pH in the stomach and then may release their encapsulated ingredients in the following higher pH region of the small or large intestines. Such materials may include shellac, zein, methyl acrylate-methacrylic acid copolymers, cellulose acetate phthalate (CAP), hydroxypropyl methyl cellulose phthalate, hydroxypropyl methyl cellulose acetate succinate, cellulose acetate succinate, sodium alginate, polyvinyl acetate phthalate (PVAP), methyl methacrylate-methacrylic acid copolymers, or cellulose acetate trimellitate.

Shellac in various forms may be useful in enhancing stability of various primary ingredients and may, for example, improve the water-proofing of a primary ingredient or protect lipids and other agents to collectively improve the stability of the encapsulated primary ingredient during manufacturing and storage. Shellac may be provided in solution form such as in ethanol or other solvents, including alkaline aqueous solutions such as those comprising ammonia or ammonium salts in which the solvent(s) can then be removed in spray drying or drying with fluidized bed application or application by other means. When ammonium salts were used to prepare the shellac solution, after drying the salts can subsequently be heated to release ammonia as a gas that can then be separated from the encapsulated primary ingredient.

The shellac may be neat shellac or shellac in combination with other materials apart from the solvent, such as alginate (e.g., alginate with calcium ions), whey protein or other proteins such as zein, cellulosic or other polysaccharide materials including nanofibers such as nano cellulose, or nanospun fibers. Such added materials can affect the porosity, diffusivity, or pH sensitivity, of the shellac to best fit a desired release profile for a primary ingredient. In some cases, an agent such as sorbic acid is combined with shellac that can promote disintegration when in the GI tract. The ratio of sorbic acid or other additive to the shellac on a solvent-free basis may be from 0.3% to 15%, from 0.5% to 10%, or from 1% to 8%. Similar principles apply for any additives relative to dry coating materials for primary, secondary, or other coatings.

Shellac may be used over one or more coating layers that are susceptible to elevated temperature to hold them in place until cooled. Thus, a lipid layer may comprise a first or second layer on a primary ingredient and a shellac layer may be part of an outer layer that helps protect the internal materials when at elevated temperature, such that they are held in place rather than separating from the primary ingredient when molten in a relatively high-temperature matrix solution.

Shellac tends to be a weak acid that has a pH-dependent dissolution profile. Dissolution tends to be low at low pH, where it normally may be effective in maintaining integrity and preventing release of ingredients in stomach acids. Dissolution may proceed more rapidly at a pH of about 7, such as at 7.3 or above. In some embodiments, shellac is selected that has a predetermined acid value, such as an acid value of at least 60, 65, 70, or 75. Higher acid values may be useful in some aspects when increased stability during storage is needed or increased resistance to stomach acids is desired. However, in some aspects, a lower acid value level may be useful in achieving a desired release profile or in terms of computability with the materials it contacts. Thus, in some aspects, a shellac is applied as a coating layer for a primary ingredient or other portions of a chewable composition, including an outer layer of the entire chewable composition or an amount of a gelled material, wherein the acid value is less than 70, 65, 60, 55, or 50, or from 40 to 70, 40 to 65, 40 to 60, 40 to 55, or 30 to 50. Acid value is measured according to the European Pharmacopoeia (Ph. Eur.), with ground shellac dissolved in a 1:1 mixture of diethyl ether and ethanol and titrated with 0.1 M potassium hydroxide solution.

Whether shellac solution is applied in a water-ethanol solution, an alkaline (e.g., ammonia) solution, or in another solvent system, it may be combined with additional agents that may acts as pore formers, plasticizers, or strength modifiers, including, for example, one or more of hydroxypropyl methylcellulose (HPMC), triethyl citrate (TEC), adipic acid, alginic acid, benzoic acid, citric acid, fumaric acid, sorbic acid, gelatin such as gelatin type B, agar, gums such as xanthan gum, lactose, proteins such as zein or casein, salts of any of the aforementioned acids such as sodium citrate or sodium alginate, a starch, cyclodextrin, or maltodextrose.

With respect to any one of the encapsulated materials in a chewable composition, the encapsulated ingredients may individually be designed to function as: (1) Extended-release products. These may allow, for example, a significant (e.g., twofold or greater) reduction in dosage frequency of a primary ingredient compared to being provided as an immediate-release (conventional) product. Extended-release dosage forms may include controlled-release, sustained-release, and long-acting primary ingredients; (2) Delayed-release products. A dosage form which may include enteric-coated primary ingredients, wherein the product releases a discrete portion or portions of a primary ingredient at a time well after administration (e.g., after swallowing, such as at least 2 minutes after swallowing, or at least 10, 30, 50, or 100 minutes after swallowing). An initial portion may be released promptly after administration; (3) Immediate release products which release the primary ingredient immediately upon being ingested or shortly thereafter (e.g., within less than 2 minutes, thereby making the primary ingredient available in the mouth, throat, and/or stomach); (4) Targeted-release primary ingredients that release a primary ingredient at or near the intended physiologic site of action and may include delayed or extended-release dosage forms; (5) Biphasic or multiphasic release, wherein the ingredient is released in two or more phases, such as an initial immediate release phase followed by a sustained release phase. This may be useful for drugs that require both a quick onset of action and sustained therapeutic effect; (6) Chronotherapeutic or pulsatile release, wherein an ingredient is released in response to rhythms of the body including circadian rhythms or is released in pulses that may be related to rhythms of the body; and/or (7) Responsive release. Here the ingredient is released in response to a specific trigger, such as a change in pH, temperature, or the presence of a specific molecule.

Encapsulation via coacervation may be accomplished in a variety of ways. A modified emulsion may be prepared in which particles or molecules of a primary ingredient are encased by coating materials, often formed by interaction of oppositely charged colloids or other interactions driven by changes in parameters such as temperature, pH (e.g., by lowering the pH of the emulsion below the isoelectric point of a protein or other macromolecule), or composition. Water may then be removed, if desired, from the coacervate. Collected particles may be dried and then further treated via other encapsulation methods.

For example, protein-based methods may be used with proteins such as gelatin, whey, casein, collagen, soy protein, lentil protein, pea protein, zein and other plant proteins. Colloidal protein having a charge (e.g., a positive charge) may be combined with an aqueous solution, suspension, emulsion, colloid, or slurry having molecules or particles of a primary ingredient, and then an oppositely changed (e.g., negatively charged) colloid such as a polymer including polysaccharides, such as at least one or the group consisting of carboxymethylcellulose, sodium hexametaphosphate, gum arabic, agar gum, pectin, gellan gum, xanthan gum, or gum tragacanth, may be combined with the mixture. The interaction of the oppositely charged materials may yield a coacervate that encapsulates the primary ingredient. During or after coacervation, an enzyme may be used to cross-link the protein. For a primary ingredient that is not a particulate but a liquid such as an oil (e.g., fish oil, avocado oil, jojoba oil, or essential oils), an oil-water emulsion may be formed and the protein-based method of encapsulation may be used to encapsulate the oil. The water may then be removed, if desired.

Charged materials that may be used to form a coacervate also include tannic acid, gum arabic, pectin, and chitosan. Materials that may crosslink polymers in a coacervate or otherwise harden the coacervate include tannic acid, tannins, polyphenols such as those from plants, transglutaminase, genipin, hydroxycinnamic acid, citric acid, phytic acid, ferulic acid, or tannic acids.

Other additives and excipients that may be helpful in coacervate production and in ensuring desired product quality may include surfactants such as Tween surfactants, natural surfactants, ionic and anionic surfactants; emulsifiers such as lecithin, oleates and polysorbates; plasticizers such as glycerin or sorbitol; cellulosic materials such as nanocellulose, microfibrillated cellulose (MFC), ethyl cellulose and other cellulose derivatives, and carboxymethylcellulose; antioxidants such as ascorbate salts or ascorbic acid; acids or bases or buffering agents to modify pH including citric acid, citrates, or calcium carbonate; chelants; and phosphates such as monophosphates or polyphosphates such as sodium hexametaphosphate, triphosphate salts, or diphosphate salts.

When the coacervate is formed, water may be removed by: (1) draining or straining, which may remove 20-60%, for example, of the water as free supernatant water; (2) pressing with a filter press, drum press, flatbed press, at elevated pressure such as from 5 bar to 100 bar or from 5 to 50 bar or, in some aspects, less 15, 10, or 5 bar; (3) centrifugation applying, for example, from 5,000 to 20,000 g may be used to remove more water, but will compact the coacervate, which may not be desired in some aspects; (4) vacuum filtration; (5) thermal treatment such as the application of hot air (e.g., spray drying), steam, radiant heat, or microwaves to heat the coacervate to a temperature that allows significant evaporation to occur, such as from 40° C. to 130° C., 40° C. to 120° C., 30° C. to 110° C., 35° C. to 97° C., 35° C. to 97° C., 40° C. to 85° C., 40° C. to 90° C., 40° C. to 70° C., or 35° C. to 65° C. or 35° C. to 60° C. (vacuum may also be applied); (6) freeze drying.

In some aspects, such water removal methods may be used to yield a relatively dry material having under 20% moisture such as from 1 wt % to 20 wt %, 2 wt % to 19 wt %, 3 wt % to 18 wt %, 4 wt % to 17 wt %, 5 wt % to 16 wt %, 6 wt % to 15 wt %, 7 wt % to 14 wt %, 8 wt % to 13 wt %, 9 wt % to 12 wt %, or 10 wt % to about 11 wt %. However, in some aspects, the moisture after moderate draining or pressing may not be removed but may be provided as part of the coacervate as it goes on to chewable composition manufacturing, where it may be incorporated into or attached to the chewable composition matrix by one of several ways: (1) blending into the hot aqueous matrix solution prior to pouring the aqueous matrix solution into molds, whereby a substantial amount of the moisture may be removed by evaporation (e.g., 5% of more, 10% or more, or 20%, 30%, 50%, or 60% or more), (2) blending with a lipid or other material such as a chocolate, viscous cream, or frosting, for example, suitable to serve as a medium in a encapsulating complex (which may be further coated or remain without an additional coating) to hold the coacervate and be placed within or on a chewable composition matrix, either by blending the encapsulating complex into the aqueous matrix solution, adding to a layer of aqueous matrix solution in a mold after the aqueous matrix solution has begun to cool, embedding it within the aqueous matrix solution in a mold or within a partially cooled or gelled chewable composition matrix, or applying or attaching the encapsulating complex to a surface of the chewable composition matrix; (3) attaching it to a surface of the chewable composition matrix or a chewable composition having a chewable composition matrix.

In some aspects, a solid-in-oil-in-water (S-O-W) emulsion may be used to provide drugs, probiotics, proteins, and other agents in encapsulated form for enhanced stability. A solid is added to an oil phase, which forms a solid-oil dispersion, that is then combined with water to form an emulsion. With the aid of further stabilizing agents, if desired, such as cellulose derivatives, nanocellulose, micro clays or other mineral particulates such as bentonite or magnesium oxide, the emulsion may be stabilized for partial removal of water, or may be used as is.

Flavor masking methods for flavor or aroma masking may include approaches such as microencapsulation, complexation, and hot melt. Microencapsulation methods can include chemical polymerization, physicochemical microencapsulation (e.g., using a film-forming composition to encase the primary ingredient), and mechanical microencapsulation. Chemical polymerization can involve a monomer in liquid that polymerizes at the interface of a primary ingredient to encapsulate it. This can occur via coacervation, in which the solubility of a polymer may be decreased by addition of a salt or other material, a change in pH or temperature, causing the polymer to precipitate on and around particles of the primary ingredient. Another approach is ionotropic gelation in which a polymer solution also containing the primary ingredient is formed into droplets which are added to a solution containing ions that can cross-link the biopolymer, causing it to form a gel.

Mechanical encapsulation typically involves applying a liquid or slurry of a coating material to particles of the primary ingredient and then removing a solvent to leave a solid or gelled coating on the primary ingredient. This may be done using fluidized bed coating, solvent evaporation/extraction, or spray drying.

Complexation may include the use of host molecules that can form inclusion complexes with guest molecules, effectively encapsulating them and altering their physical and chemical properties. This may be used to mask unpleasant tastes or odors, enhance stability, and control the release of primary ingredient. Useful agents for complexation can include cyclodextrins such as alpha-, beta, and gamma-cyclodextrins as well as derivatives of cyclodextrins such as hydroxypropyl-beta-cyclodextrin or triacetyl-beta-cyclodextrin. Other complexing agents include cucurbiturils such as cucurbit[n]uril or CB[n], where n is the number of glycoluril units that can range from 5 to 12, but typically is 6, 7, or 8. Cucurbiturils derivatives may also be considered such as hydroxypropyl cucurbit[n]uril or methylated cucurbit[n]uril. In some aspects, depending on the nature of the primary ingredient or the aromas of concern, chelants can also be useful as complexing agents, such as citric acid or citrates (e.g., sodium, potassium), EDTA, phytic acid, and phosphates such as polyphosphates, including sodium hexametaphosphate.

In some aspects, the treatment or microencapsulation applied to a primary ingredient may not be a solid, impervious coating but may include a complexing agent or a hydrogel.

Hot melt methods, also known as hot melt extrusion (HME), involves combining a primary ingredient with a meltable binder that melts at a relatively low temperature at which the primary ingredient is not melted or damaged. Such materials may include, for example, lipids such as fats or waxes and polymers such as methyl and ethyl methacrylate anionic based copolymer (e.g., Eudragit L100 and Eudragit L100-55). The binder used in the present invention is a meltable material that is pharmaceutically acceptable. Suitable binders may include, but are not limited to, polymers such as polyethylene glycol (PEG), polyvinylpyrrolidone (PVP), and hydroxypropyl methylcellulose (HPMC), or lipids such as glyceryl monostearate or glyceryl behenate. The primary ingredient may be any pharmaceutical compound that is stable under the processing conditions and may be dispersed within the binder.

A slurry or paste may be made and extruded into particles that, when cooled, comprise a solid phase in which the primary ingredient is embedded. The particles of the complex may then be further milled, refined, or classified to provide the right particle size range for a chewable composition. Hot melt extrusion materials may be further coated by other means, such as spray drying to impart a relatively temperature resistant coating on the complex provided by hot melt extrusion. A plasticizer may enhance the processing and in turn result in better taste masking performance. In some cases, ionic interaction between the meltable material and the primary ingredient can lead to enhanced taste suppression.

One method for forming a hot melt extrusion is twin-screw extrusion. Industrial-scale hot melt extrusion typically involves the use of a twin-screw extruder. The twin-screw extruder includes a feeding zone, a compression zone, and a metering zone. Additional equipment may include a feed hopper for introducing the matrix material and primary ingredient into the extruder, a heating system for melting the matrix material, and a cooling system for solidifying the extruded product.

In some aspects, primary ingredients may be provided in solid or powder form and may be further processed in grinding or attrition devices such as a cone mill, pin mill, disk refiner, granulator, powder mill, colloid mill, or cone blender. Suitable particle size ranges may be selected from the milled materials through sieving, wire sifting, aerodynamic sorting, and/or centrifugal sorting. The particles may then be entrained in an air or other fluid stream (including liquids and slurries) or otherwise conveyed into an encapsulating system to be given one or more coatings or other treatments.

Any suitable encapsulation method may be used. Encapsulation methods include but are not limited to: (1) Fluidized bed coating. Fluidized bed coating may include Wurster coating or fluid bed microencapsulation, in which particles in a fluidized bed are carried by an air flow to create a cyclic movement of material with one or more spray nozzles at the bottom of the fluidized bed of particles (or elsewhere), coating particles with an atomized material to create a core-shell structure; (2) Spray drying: In this method, a solution or suspension of the drug and a coating material is sprayed into a hot chamber. The solvent evaporates, leaving behind small particles of the drug coated with the material. The resulting powder can then be incorporated into the chewable composition formulation. Spray drying may include partly drying a composition in a spray drying tower, wherein the composition includes a suspension and an emulsion of a primary ingredient and an aqueous solution of a coating material at an air temperature of about 70° C. to 200° C., or from 90° C. to 180° C., or from 80° C. to 160° C., to form partly dried particles (e.g., having a semi-solid skin), and further drying the particles at an air temperature between 0° C. to 70° C. such as from 30° C. to 60° C. to yield substantially dry particles with coating layers; (3) Coacervation. This may include the separation of a solution into two liquid phases, one of which forms a coating around the drug particles. The coating is then hardened to form the microcapsules; (4) In-situ polymerization. A primary ingredient may be coated with a coating material that polymerizes directly on the surface of the primary ingredient (e.g., particles). Polymerization may be driven by UV light, by an initiator such as a peroxide (e.g., a hydrogen peroxide spray in the fluidized air stream as the products are being treated or transported), by temperature; (5) Solvent evaporation: In this method, the primary and a coating material are dissolved in a common solvent, which is then evaporated to form microcapsules; (6) Extrusion may be used in processes such as alginate encapsulation, which is commonly achieved by nozzle extrusion and external gelation. Using this method, primary ingredient-containing alginate droplets formed at the tip of nozzles fall into a solution containing divalent cations that cause ionotropic alginate gelation as they diffuse into the droplets. Alginate may be used alone or in combination with other agents such as gelatin, pigment particles, silica materials, cyclodextrin, or a shellac coating, to improve its flavor masking capability or for other effects. The use of cyclodextrin, for example, may assist in both flavor and odor masking; (7) Hot melt extrusion (HME) may be used to encapsulate primary ingredients within a meltable binder. The binder is generally a pharmaceutically acceptable meltable material that may include methyl and ethyl methacrylate anionic based copolymer (e.g., Eudragit L100 and Eudragit L100-55), polyethylene glycol (PEG), polyvinylpyrrolidone (PVP), and hydroxypropyl methylcellulose (HPMC), or lipids such as waxes, solid or semi-solid fats, glyceryl monostearate or glyceryl behenate. The primary ingredient may be any pharmaceutical compound that is stable under the processing conditions and may be dispersed within the binder. A plasticizer may enhance the processing and in turn result in better taste masking performance. In some cases, ionic interaction between the meltable material and the primary ingredient can lead to enhanced taste suppression. The extruder may be a twin screw extruder or other types such as gear pump extruders, multi-screw extruders, single-screw extruders, or ram extruders. The binder and the primary ingredient may be fed into the feed hopper of an extruder, heating the materials to melt the binder and form a uniform dispersion or bend with the primary ingredient, extruding the mixture through the die of the extruder to form a thin strand, cooling the extruded strand to solidify the binder and encapsulate the primary ingredient, and cutting, grinding, or milling the solidified strand into particles of the desired size range, which may then be further refined or classified as desired. The encapsulated primary ingredient from hot melt extrusion may be further coated by other means, such as spray drying, to impart a relatively temperature resistant coating on the complex provided by hot melt extrusion; or (8) Electrospinning to apply fine fibers such as nanofibers to cover all or part of the surface of a particle with a suitable material such as a polysaccharide or polymer. Electrospinning as a method for encapsulation may include electrohydrodynamic atomization of the primary ingredient during the electrospinning of fibers which may be nanofibers, or otherwise combining a primary ingredient with electrospun fibers by coating, entanglement, electrostatic attraction, adhesion, coating, or embedding. Electrospun materials tend to have large surface area and 3D continuous structure that may be manipulated to create broad ranges of filament widths, mesh geometry, fabric density and porosity. Electrospinning approaches may include, by way of example, airblowing-assisted, multi-nozzle, pressurized gyration, needleless, corona, and centrifugal spinning techniques. Melt spinning may be done at lower temperatures than is typical for spray drying or other techniques, including at room temperature or slightly elevated temperatures, and often avoid the temperature ranges that may be harmful for some primary ingredients. From this point of view, it has been thought that the electrospinning might be also conducted for the efficient encapsulation of essential oils into nanofibrous webs.

One or more of the above encapsulation methods may be applied sequentially to add multiple layers of coating materials to primary ingredient particles. For example, in a Wurster coater, particles treated with a first encapsulant may be dried and then circulated in the same or a different fluidized bed and treated with a second encapsulant, and this may be repeated as many times as desired to apply multiple layers to particles of primary ingredients. As a further example, a first coating may be applied with one method such as a spray dryer, and a second layer may be applied by a different method such as a fluidized bed, and a third layer may be applied by electrospinning or, for example, by solvent evaporation to apply a material such as shellac From a Solution in Ethanol or Other Solvents.

### Chewable Composition Manufacturing

Further provided herein are methods of producing a chewable composition having a Fast Melt texture. The methods generally comprise combining water, a hydrocolloid, and one or more bulking agents to form a gel matrix; combining the gel matrix with a primary ingredient to form a mixture; heating the mixture to a temperature of at least 70° C., forming the mixture into chewable units; and allowing the chewable units to age for a period of 2 to 4 weeks under ambient conditions to form the chewable composition. The chewable composition may be any chewable composition described herein.

In some embodiments, the method may generally comprise combining water, a hydrocolloid, and one or more bulking agents to form a gel matrix; heating the gel matrix to a temperature of at least 70° C., combining the gel matrix with a primary ingredient to form a mixture; forming the mixture into chewable units; and allowing the chewable units to age for a period of 2 to 4 weeks under ambient conditions to form the chewable composition. The chewable composition may be any chewable composition described herein.

Combining the water, hydrocolloid, and the bulking agent(s) may be accomplished by adding the hydrocolloid and the bulking agent(s) to a vessel and mixing the contents. All ingredients may be added to a vessel before mixing begins, or the ingredients may be added sequentially as mixing is conducted, or the ingredients may be added simultaneously as mixing is conducted. The mixing apparatus may be equipped with mixing, heating, and cooling capabilities. Mixing apparatuses known in the art may be used to mix the ingredients to form a homogeneous mixture; for example, a stand mixer, a paddle mixer, ribbon blender, planetary mixer, high shear mixer, or any other mixing apparatus known in the art. The mixing time may take place for a period from about 15 seconds to about 30 minutes. The mixing may be high shear mixing or low shear mixing. The ingredients may be provided in solid, liquid, or slurry form and combined with water.

The combining may be conducted at ambient conditions or at an elevated temperature. For example, the mixing may be conducted at a temperature from about 20° C. to about 65° C., such as from about 20° C. to about 30° C., about 20° C. to about 40° C., about 20° C. to about 50° C., about 20° C. to about 60° C., about 20° C. to about 65° C., about 30° C. to about 65° C., about 40° C. to about 65° C., about 50° C. to about 65° C., about 30° C. to about 50° C., or about 40° C. to about 60° C. As another example, the temperature may be about 20° C., 25° C., 30° C., 35° C., 40° C., 45° C., 50° C., 55° C., 60° C., or about 65° C.

Once the gel matrix is formed, the gel matrix is combined with a primary ingredient to form a mixture. The primary ingredient may be added as mixing continues or mixing may be briefly stopped while the primary ingredient is added before resuming. After the primary ingredient is added, the mixing time continues for a period from about 30 seconds to about 30 minutes. The mixing may be high shear mixing or low shear mixing.

After the mixture is formed, the mixture is heated to a temperature of at least 70° C. The mixing may continue during this step. For example, the mixture may be heated to a temperature of at least 70° C., at least 80° C., at least 90° C., at least 100° C., or at least 110° C. In some embodiments, the mixture is heated to a temperature from about 85° C. to about 120° C., such as from about 85° C. to about 90° C., about 85° C. to about 100° C., about 85° C. to about 110° C., about 85° C. to about 120° C., about 90° C. to about 120° C., about 100° C. to about 120° C., about 110° C. to about 120° C., or about 90° C. to about 110° C. As another example, the mixture may be heated to a temperature of about 85° C., 90° C., 95° C., 100° C., 105° C., 110° C., 115° C., or about 120° C. Once heated, the mixture may be held at the heated temperature for a period of up to about one hour. Mixing may continue during this time to ensure the mixture is evenly heated and homogeneous. The heating may be accomplished using apparatuses known in the art. For example, a jacketed vessel may be used to heat the homogeneous mixture.

Once the heating step is complete, the mixture is formed into chewable units. This step may be accomplished by pouring the mixture into molds, which conform to a desired shape and size of the chewable composition. The homogeneous mixture may be deposited into the molds directly from the vessel in which the homogeneous mixture was formed, or aliquots of the homogeneous mixture may be removed from the vessel sequentially and deposited into separate molds. The molds may be silicone molds or another food-safe or pharmaceutical-grade material.

Once deposited into a mold, the mixture is cooled to below the gel temperature of the hydrocolloid. Methods for determining the gel temperature of the hydrocolloid are known in the art, and those having ordinary skill in the art are capable of determining the gel temperature of the hydrocolloid chosen. The cooling may be accomplished in ambient conditions, or the molds holding the mixture may be placed in a cooling apparatus (e.g., a refrigerator or freezer) to expedite the cooling process or cool the mixture below ambient temperature.

In some embodiments, the mixture may be deposited into a starch molding machine such as a Mogul. The starch employed helps prevent the chewable units from sticking to the chewable composition molds, and may help hold the chewable units in place during the drying, cooling, and setting processes, and may absorb moisture from the candies to help give them the desired texture. The chewable units are prepared by filling molds in the Mogul device, wherein the molds have starch applied before receiving the flowable material. After being formed and cooled in the molds, the starch on the surface of the chewable units can reduce tackiness and modify the surface feel and mouth feel as the starch absorbs moisture and more fully bonds with the matrix. In one aspect, hot air may be further applied to prepare the surface of the chewable compositions to interact with the starch or other materials added to the surface.

Once cooled, the mixture sets to form chewable units. The chewable units can then be removed (e.g., by inverting trays to dump chewable compositions onto a moving wire or vibrating metal screen that can act as a sieve) to remove excess starch and expose the chewable compositions to various surface treatments (sprayed with vegetable oil, sprinkled with sugars, tartness agents such as tartaric or citric acid, waxes such as bees wax or carnauba wax, or other solids). At this point, a coating may be applied to the chewable units. The coating may be a sugar coating or other commonly used coating ingredient. Methods for coating the chewable units are generally known in the art, such as hard panning, soft panning, powder panning, perforated-drum film coating, glazing and polishing, Wurster coating, fluid-bed coating, spouted-bed coating, dip coating, and the like.

The chewable units are then aged for a period from about 2 weeks to about 4 weeks to form the chewable composition having a Fast Melt texture. The aging period may be accomplished either in the facility where the chewable units are formed, or the chewable units may be packaged and transported for commercial use during the aging period. Notably, aging may also occur after chewable units have been packed into bottles. Importantly, the aging period is completed prior to use or consumption by a consumer.

The conditions of the aging step are not particularly limited, so long as the conditions are not so extreme (e.g., excessively high temperature or humidity) such that they cause the chewable composition to decompose or otherwise be damaged.

In another aspect, a chewable composition is prepared by mixing water at elevated pH, gelatin, pectin, carrageenan and/or agar, sweeteners such as sugar and/or glucose syrup, or, for sucrose-free chewable compositions, polyols and/or polydextrose, then adding colorant, flavorings and aromas, buffers, to form a mixture capable of gelling, then adding one or more kinds of encapsulated or unencapsulated actives and other functional ingredients while the mixture at a predetermined blending temperature that may be between 1° C. and less than 100° C., such between 5° C. and 95° C., 10° C. and 90° C., 15° C. and 85° C., 20° C. and 80° C., 25° C. and 75° C., 30° C. and 70° C., 35° C. and 70° C., 40° C. and 65° C., 45° C. and 60° C., or 50° C. and 55° C., and adding pH control agents to achieve a predetermined pH, then depositing the blended mixture on molds that may optionally have a particulate base such as starch or other polysaccharides. The filled molds may then be dried and removed from the mold.

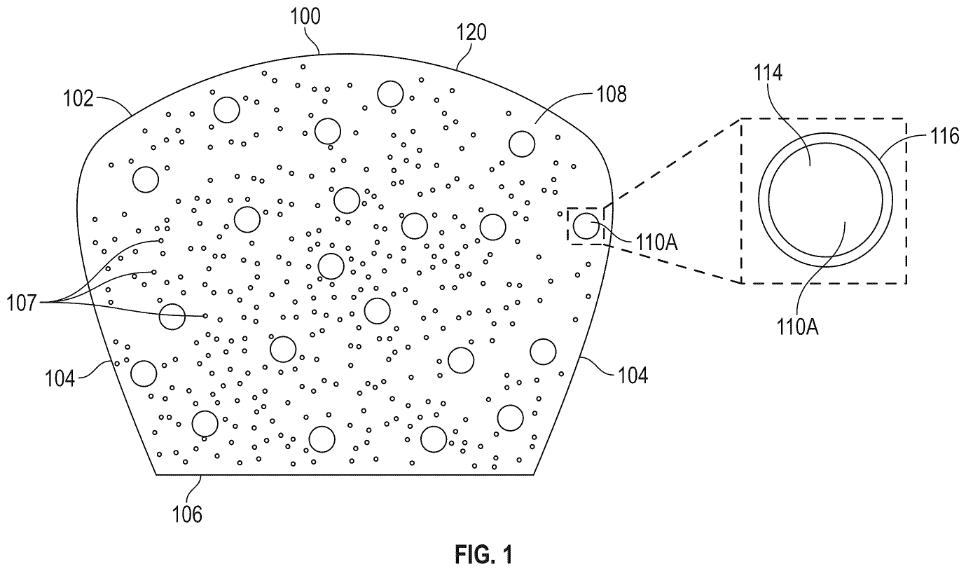

FIG. 1 depicts a cross-section of a chewable composition 100 having a chewable composition matrix 108 with an outer surface 102 with a top surface 120, side surfaces 104, and a bottom surface 106. The chewable composition matrix 108 has an elevated pH such as 6.5 or higher, or 7.5 or higher. In the interior of the chewable composition matrix 108 is a first primary ingredient 107 shown as dispersed particles relatively uniformly distributed in the chewable composition matrix 108. The first primary ingredient 107 may be an acid-sensitive ingredient such as a calcium salt, present as a particulate as depicted, or as a solute distributed at a molecular level (not shown) within the chewable composition matrix 108. An optional acid-sensitive encapsulated second primary ingredient 110 is also shown, depicted as a plurality of similar coated spheres. A characteristic acid-sensitive encapsulated second primary ingredient 110A is shown with an enlarged view, revealing the second primary ingredient 114 and its first coating layer 116, which may be a multilayer or multicomponent coating including polymers, lipids, nanofibers, shellac, and the like.

In some embodiments, a hydrocolloid having from 40% to 100% carrageenan, such as a mixture of kappa, lambda, and iota carrageenans, and up to 60% of other hydrocolloids such gelatin A, gelatin B, fish gelatin, porcine gelatin, agar, alginate, pectin, or gum Arabic may be prepared by dispersing the hydrocolloids into water having a pH above 3 and hydration-inhibiting ions with an effective concentration of a compound such as magnesium citrate, calcium gluconate, potassium ascorbate, and/or sodium hexametaphosphate. An acid-sensitive calcium supplement or antacid may be present in a concentration of from 60 mg/g to 300 mg/g of chewable composition matrix. A second active ingredient may be present such as simethicone at a dose of from 50 mg/g to 100 mg/g of chewable composition matrix. Tween surfactant and lecithin as an emulsifier may be present. The chewable composition may be adapted to deliver both an antacid benefit and a reduction in flatulence or stomach discomfort. The pH of the chewable composition matrix in the absence of the acid-sensitive calcium supplement or antacid may be 3 to 11 or 10.5, and the direct pH of the finished product when cut open and tested with a suitable flat electrode may be from 6.8 to 11. The resulting product meets key characteristics of performance.

In some embodiments, the chewable composition may include water, sugar, corn syrup, carrageenan, and sanding sugar. The chewable composition may further include glycerine, a high-intensity sweetener, taste-masking materials, soy lecithin, coconut oil, insoluble solids, flavor (such as salt), an active pharmaceutical ingredient, color, or any combination thereof.

The chewable composition may include water in an amount ranging from about 15% to about 20% by weight of the chewable composition. The chewable composition may include sugar in an amount ranging from about 30% to about 52% by weight of the chewable composition. The chewable composition may include corn syrup in an amount ranging from about 9% to about 26% by weight of the chewable composition. The chewable composition may include carrageenan in an amount ranging from about 0.75% to about 1.0% by weight of the chewable composition. The chewable composition may include glycerine in an amount ranging from about 0% to about 5% by weight of the chewable composition. The chewable composition may additionally include sanding sugar (e.g., external sugar coating) in an amount ranging from about 2% to about 5% by weight of the chewable composition. The chewable composition may include a high-intensity sweetener in an amount ranging from about 0% to about 0.02% by weight of the chewable composition. The chewable composition may include taste-masking materials in an amount ranging from about 0% to about 2% by weight of the chewable composition. The chewable composition may include emulsifiers such as soy lecithin in an amount ranging from about 0% to about 0.2% by weight of the chewable composition. The chewable composition may include coconut oil in an amount ranging from about 0% to about 4% by weight of the chewable composition. The chewable composition may include insoluble solids in an amount ranging from about 0% to about 20% by weight of the chewable composition. The chewable composition may include flavor in an amount ranging from about 0% to about 0.8% by weight of the chewable composition. The chewable composition may include active pharmaceutical ingredient (API), such as calcium carbonate, in an amount ranging from about 0% to about 20% by weight of the chewable composition. The chewable composition may include color in an amount ranging from about 0% to about 1% by weight of the chewable composition.

The chewable composition may include water in an amount ranging from about 12% to about 35% by weight of the chewable composition. The chewable composition may include sugar in an amount ranging from about 20% to about 60% by weight of the chewable composition. The chewable composition may include corn syrup in an amount ranging from about 5% to about 40% by weight of the chewable composition. The chewable composition may include carrageenan in an amount ranging from about 0.5% to about 4.0% by weight of the chewable composition. The chewable composition may include glycerine in an amount ranging from about 0% to about 10% by weight of the chewable composition. The chewable composition may additionally include sanding sugar (e.g., external sugar coating) in an amount ranging from about 2% to about 5% by weight of the chewable composition. The chewable composition may include a high-intensity sweetener in an amount ranging from about 0% to about 0.5% by weight of the chewable composition. The chewable composition may include taste-masking materials in an amount ranging from about 0% to about 2% by weight of the chewable composition. The chewable composition may include soy lecithin in an amount ranging from about 0% to about 1% by weight of the chewable composition. The chewable composition may include coconut oil in an amount ranging from about 0% to about 10% by weight of the chewable composition. The chewable composition may include insoluble solids in an amount ranging from about 0% to about 35% by weight of the chewable composition. The chewable composition may include flavor in an amount ranging from about 0% to about 5% by weight of the chewable composition. The chewable composition may include active pharmaceutical ingredient (API), such as calcium carbonate, in an amount ranging from about 0% to about 35% by weight of the chewable composition. The chewable composition may include color in an amount ranging from about 0% to about 5% by weight of the chewable composition. The chewable composition may include flavor masking agents in an amount ranging from about 0% to about 0.18% by weight of the chewable composition.

### Terms and Definitions

When introducing elements of aspects of the invention or the embodiments thereof, the articles “a,” “an,” “the,” and “said” are intended to mean that there are one or more of the elements, and thus may include plural referents unless the context clearly dictates otherwise. The terms “comprising,” “including,” and “having” are intended to be inclusive and mean that there may be additional elements other than the listed elements.

As used herein, the word “exemplary” means serving as an example, instance, or illustration. The aspects described herein are not limiting but rather are exemplary only. It should be understood that the described aspects are not necessarily to be construed as preferred or advantageous over other aspects. Unless otherwise indicated, no aspect of any invention described herein should be assumed to have the same advantages or features had by any other aspect.

As used herein, the term “acid-sensitive” refers to substances that are increasingly soluble or reactive at low pH thus leading to instability and release of unpalatable taste on exposure to low pH matrix in a gummy during manufacturing processes.

As used herein, the term “palatability” means the overall appreciation of an oral medicinal product in relation to its smell, taste, aftertaste, and feeling in the mouth. It is a measure of how pleasant or acceptable a medicine is to patients, based on its smell, taste, and overall sensory experience. Palatability refers to the overall acceptability of the chewable composition to the consumer, with a particular emphasis on its sensory attributes. This encompasses not only the taste, which prioritizes the minimization or masking of bitter flavors, but also extends to other sensory experiences such as odor, aftertaste, texture, and mouthfeel. Improving palatability may be achieved through the addition of flavor enhancers, bitter blockers, sweeteners, or other flavor masking agents, or through encapsulation or complexation of the active ingredient.

As used herein, the term “stability” refers to the amount of change a product's physical or chemical or sensorial attributes go through over a defined period of time and at set environmental conditions. The product is said to be “stable” if the physical, chemical, and sensorial changes are within a quantitative range deemed acceptable as decided by product developers.

As used herein, “rapid disintegration” or “rapidly disintegrating” means achieving substantially complete disintegration under masticated conditions in a time of less than 120 seconds, less than 50 seconds, or less than 10 seconds. Rapid disintegration may be used to determine dissolvability of the chewable composition. Substantially complete disintegration can mean de-aggregation, dispersion, or dissolution of the core to an extent that no portion of the core remains as a solid monolith compact having a size that is greater than 50%, greater than 10%, greater than 5%, or greater than 2% of the size of the original core.

As used herein, “chewable” refers to a final product exhibiting a hardness level that allows for chewing without irritation or negative impact to the consumer. A product having an acceptable hardness that poses no difficulty for the consumer to utilize his/her teeth to break the initial fully intact product.

As used herein, “suitable release profile” refers to, but are not limited to instant release profile or immediate release profile, sustained release profile or extended release profile, and delayed release profile. The release profile is affected by the drug and/or drug formulation to be administered (e.g., coatings, specific excipients, and other drug product attributes), the concentration of drug in the formulation, the medium for which the release or dissolution of the product takes place, and a variety of other factors that are appreciated by those of ordinary skill in the art.

As used herein, an “instant release profile” or an “immediate release profile” refers to a release profile wherein no less than about 80% of the API is released in 60 minutes in vitro as measured in a USP approved Type 2 paddle apparatus at 50 rpm or 100 rpm at a constant temperature of 37±0.5° C.

As used herein, a “sustained release profile” or an “extended release profile” refers to a release profile wherein no more than about 80% of the API is released within about 6 hours in vitro as measured in a USP approved Type 2 paddle apparatus at 50 rpm or 100 rpm at a constant temperature of 37±0.5° C.

As used herein, a “delayed release profile” refers to a release profile wherein, dependent upon the number of units tested, the average cannot exceed 10%, and no individual unit can exceed 25% of the API released during the acid stage, performed using 0.1 N HCL for 2 hours, as tested in vitro and measured in a USP approved apparatus at a constant temperature of 37±0.5° C. After completion of the acid stage, each unit should have, at least, 75% of the initial API dose remaining and available for release in a USP buffer media, pH 4.5-7.5. During the buffered stage, a multipoint dissolution profile should be obtained using adequate sampling times such as, but not limited to, 15, 30, 45, 60, and 120 minutes resulting in the release of no less than 80% of the initial, pre acid stage, API dose at a constant temperature of 37±0.5° C.

As used herein, “solid sweeteners” refer to crystalline or powdered substances used to impart sweetness to chewable compositions. They may be added in their dry/solid state and may also contribute to the texture of the formulation. Examples include but are not limited to natural sugars (like sucrose, glucose, fructose etc.), sugar alcohols (sorbitol, xylitol, mannitol, erythritol), or high-intensity sweeteners in solid form (stevia, granulated or powdered aspartame). Solid sweeteners enhance palatability by masking the bitter or unpleasant taste of primary ingredients, provide structural support to the dosage form, may contribute to mouthfeel and disintegration properties during chewing, and help maintain low moisture content which is important for stability.

As used herein, “liquid sweeteners” refer to syrups, solutions or viscous liquids used to add sweetness. Liquid sweeteners are typically incorporated in a dissolved or semi-liquid form. Examples include but are not limited to natural syrups (honey, agave syrup, maple syrup, molasses), artificial liquid sweeteners (liquid sorbitol, liquid sucralose, glycerol-based sweeteners), or syrups containing sugars (glucose syrup, corn syrup). Liquid sweeteners enhance sweetness while adding some moisture which can improve the chewability and texture of the product, may act as a plasticizer thereby helping the chewable composition remain soft & pliable and assist in achieving uniform sweetness by distributing evenly throughout the formulation.

As used herein, “functional ingredients” refer to minerals, vitamins, nutraceutical agents, or other supplements. The supplements may include derivatives, salts, and/or mixtures of the foregoing.

As used herein, “active ingredient” refers to a substance that may function as a functional ingredient, an active pharmaceutical ingredient, or a combination thereof.

As used herein, “stable” in the context of the chewable composition refers to the ability of the dosage form to maintain its physical, chemical, and microbiological properties over time under specified storage conditions. A stable chewable composition ensures that the product remains safe for consumption, and the sensory qualities (e.g., taste, appearance) are preserved until the product reaches its expiration date. Key factors contributing to stability in chewable compositions include chemical stability, physical stability, microbiological stability, taste & sensory stability and packaging stability.

As used herein, “acid-sensitive ingredient” refers to an active ingredient, coating, or encapsulated material that significantly changes its physiochemical properties when at pH<5 relative to pH>5.

As used herein, “pH responsive” refers to a substance that undergoes a noticeable change in behavior, structure, or properties in response to changes in pH (the measure of how acidic or basic a solution is). These changes may include alterations in solubility, color, shape, charge, or molecular interactions.

As used herein, “efficacy” refers to the ability of a product, such as chewable composition, to produce the desired therapeutic effect under ideal or controlled conditions. In the context of pharmaceuticals or nutraceuticals, efficacy indicates how well the active ingredient performs its intended function in treating or preventing a condition. The efficacy of a chewable composition depends on several factors including bioavailability, release profile, formulation design, and patient compliance.

As used herein, “bioavailability” refers to the extent to which the active ingredient is absorbed and becomes available at the site of action. Bioavailability refers to the proportion of a substance (such as the chewable dosage) that enters the bloodstream when it is administered and is made available for use or storage by the body. In simpler terms, it is a measure of how much of a substance reaches its site of action or target in the body, typically the bloodstream, and how efficiently it can have its intended effect.

As used herein, the term “reducing sugar” refers to a carbohydrate that possesses a free or potentially free aldehyde group or ketone group at its anomeric carbon and is therefore capable of acting as a reducing agent under typical analytical or formulation conditions. A reducing sugar may exist in equilibrium between its cyclic form and an open-chain form containing a reactive carbonyl functional group. Reducing sugars are capable of reducing mild oxidizing agents and typically yield a positive response in common analytical assays such as the Benedict's test, Fehling's test, or Tollen's test. Exemplary reducing sugars include, but are not limited to, glucose, fructose, galactose, ribose, mannose, lactose, maltose, and cellobiose.

As used herein, the term “non-reducing sugar” refers to a carbohydrate in which the anomeric carbon atoms are involved in glycosidic linkages such that no free aldehyde or ketone functional group is available to participate in redox reactions. Because the anomeric carbons are fully engaged in glycosidic bonding, non-reducing sugars are unable to convert to an open-chain form containing a reactive carbonyl moiety and therefore do not act as reducing agents. Non-reducing sugars typically do not respond in Benedict's, Fehling's, or Tollen's assays. Exemplary non-reducing sugars include, but are not limited to, sucrose, trehalose, and certain fully glycosylated oligosaccharides.

As used herein, “graining thickness” is the measured thickness of the crystalline phase that develops within the chewable gel matrix over time due to sugar recrystallization and moisture migration. Graining occurs when non-reducing sugars (and in some cases, reducing sugars) begin to form microcrystalline domains within the gel structure, typically at the outer surface or within specific inner-phase regions. Graining thickness is quantified using calipers to measure the depth of the visibly grained layer in bisected pieces. A thicker-grained layer generally correlates with increased firmness (hardness) and decreased cohesiveness, as the crystalline network provides structural reinforcement and accelerates structural fracture during mastication.

As used herein, the term “grained particles” refers to discrete solid domains formed within the bulk matrix of a chewable composition during aging, wherein the domains are dispersed throughout an otherwise continuous matrix and contribute to localized rigidity and fracture behavior that promotes fast-melt texture.

As used herein, the term “gummy” refers to a broad confectionery product type comprised of a hydrocolloid gelling agent, such as pectin, carrageenan, gelatin, etc., a boiled mixture of mixed carbohydrates, and about 12%-24% by weight of moisture. A gummy is typically soft and chewy, but can have a wide range of textures deriving from the specific nature of the formulation and manufacturing process.

As used herein, the term “pastille” refers to a type of gummy that is prepared using gum Arabic. In a pastille, the gum Arabic typically comprises up to 50% by weight of the solid composition, and produces a hard, short, somewhat malleable texture. The use of gelatin may sometimes be employed to create a softer, longer texture. Pastilles are normally prepared from gum Arabic alone which is the major ingredient constituting some 50% of all total solid matter that is present. The texture produced is hard and short, but malleable. If the level of gum Arabic is reduced to produce a softer eating product then another gelling agent such as gelatine will be required.

As used herein, the terms “chewable gel” or “chewable jelly” refer to a type of gummy typically comprising a hydrocolloid that can also be used to form a jelly or jam. This is typically pectin, carrageenan, or agar. The resulting product is non-flowable with a defined shape.

As used herein, the term “chewable bar” refers to a further defined type of gummy or chewable gel that has been formed into a regular geometric shape, typically a disc or rectangular prism.

As used herein, the term “soft chewable tablet” refers to tablets that are typically made by a molding or extrusion process, frequently with more than 10% by weight water to help maintain a pliable, soft product. Furthermore, a soft chewable tablet comprises attributes of a chewable tablet drug dosage form, which should be easy to chew, palatable, of appropriate size and shape, and able to disintegrate readily to facilitate dissolution.

As used herein, the term “panned chew” refers to a confectionery product type comprising a hard coating surrounding a chewy center. The coating may typically be made via hard panning, which involves the use of sequential application of layers of saturated engrossing syrups. The chewy center typically has low moisture with or without the inclusion of fat.

As used herein, the term “soft chew” refers to a confectionery product type comprising low moisture (less than 10%) and a syrup bulking-ingredient base. Most ingredients are low-moisture, which allows the chew to be made with or without heating. The resulting product is typically soft and pliable but may have a range of textures deriving from the specific nature of the formulation and manufacturing process. As an example, a soft chew could be extruded in order to produce a defined shape and size prior to packaging.

As used herein, the term “primary ingredient” refers to an active pharmaceutical ingredient, a dietary supplement, a food, and any combination thereof.

As used herein, the term “active pharmaceutical ingredients” or “active ingredient(s)” or “drug” refers to any therapeutically active agent. The term includes any therapeutic active agent indicated for oral administration, such as NSAIDs, antibiotics, anticoagulants and antiplatelet agents, antidiabetic agents, antiemetics, antihistamines, anti-asthmatic medications, antiarrhythmics, anticholinergics, anticholinesterase inhibitors, anticonvulsants, antidepressants, antidiabetic agents, antihistamines, antihypertensives (ACE inhibitors, alpha blockers, angiotensin II receptor blockers, beta blockers, calcium channel blockers, central alpha agonists, diuretics, and the like), anti-inflammatory drugs, antipsychotics, antipyretics, appetite suppressants, bronchodilators, chemotherapy compounds, cholesterol level medications (bile acid sequestrants, cholesterol absorption inhibitor such as ezetimibe, fibrates, PCSK9 inhibitors, statins, angiotensin II receptor blockers, beta blockers, calcium channel blockers, central alpha agonists, and the like), diuretics, expectorants, hormonal medications, laxatives, sedatives, stimulants, vasodilators, anxiolytics, mood stabilizers, antipsychotics, antidepressants and antipsychotics, and the like. The term may refer to more than one therapeutic agent, wherein exemplary combinations of therapeutic agents include a combination of two or more. The term also includes but is not limited to, minerals, vitamins, nutraceutical agents, and other supplements; including derivatives, salts (and the like), and/or mixtures of the foregoing.

As used herein, the term “hydrocolloid” refers to a hydrophilic polymeric substance, typically of natural, semi-synthetic, or synthetic origin, that disperses in water to form a viscous solution, gel, or film. Hydrocolloids include but are not limited to polysaccharides (e.g., pectin, alginates, xanthan gum, cellulose derivatives) and proteins (e.g., gelatin), and they may function as thickeners, stabilizers, film formers, or gelling agents depending on the formulation context.

Having described aspects of the invention in detail, it will be apparent that modifications and variations are possible without departing from the scope of aspects of the invention as defined in the appended claims. As various changes could be made in the above compositions, products, and methods without departing from the scope of aspects of the invention, it is intended that all matter contained in the above description shall be interpreted as illustrative and not in a limiting sense.

While the foregoing description makes reference to particular illustrative embodiments, these examples should not be construed as limitations. The inventive system, methods, and products may be adapted for other uses or provided in other forms not explicitly listed above, and may be modified in numerous ways within the spirit of the present disclosure. Thus, the present invention is not limited to the disclosed embodiments but is to be accorded the widest scope consistent with the claims below.

### EXAMPLES

### Example 1: Preparation of Fast Melt Compositions

Antacid Fast Melt compositions were formulated with calcium carbonate as the active antacid ingredient/insoluble solid. The compositions were strawberry and mixed-berry flavored. The chewable compositions were prepared as high-pH chewable gel compositions exhibiting a soft, melty texture and rapid in-mouth dissolution characteristics, achieved through the controlled incorporation of hydrocolloids (such as carrageenan) and flavoring systems. Table 1 provides the composition of the Fast Melt chewable compositions prepared. Calcium carbonate was used as the insoluble solid/API.

TABLE 1

| | Fast Melt composition | Fast Melt composition |
|---|---|---|
| Ingredient | A | B |
| Water | 10-25% | 10-25% |
| Sugar | 10-70% | 10-70% |
| Corn Syrup | 10-40% | 10-40% |
| Carrageenan | 0.5-4% | 0.5-4% |
| Emulsifier | 0-2% | 0-2% |
| Oil | 0-8% | 0-8% |
| Insoluble Solids/ API | 0-20% | 0-20% |
| Flavor | 0-2% | 0-2% |
| Color | 0-0.2% | 0-0.2% |
| Coating | 2-6% | 2-6% |

The final dosage units were disc-shaped Fast Melt chewable compositions designed to dissolve quickly with minimal mastication. Participants in sensory studies described the prototypes as “soft,” “melty,” “smooth,” and exhibiting a “marshmallow-like” texture.

### Sensory Evaluation and Focus Group Testing

Study Design: Three consumer focus groups (n=30 total) were conducted. Each group contained ten adult participants who were regular users and purchasers of over-the-counter antacid products. Groups were segmented into (1) chewy-form antacid users, (2) chalky-tablet users, and (3) mixed chewy/chalky users.

Participants sampled the chewable compositions and provided feedback on taste, texture, appearance, mouthfeel, dosage-form terminology, perceived efficacy, user preference, and perceived premium value.

### Consumer Feedback on Texture, Chewability, and Mouthfeel

Across all groups, participants consistently described the chewable compositions as: “Soft,” “very soft,” “slightly chewy,” “jelly-like,” “smooth,” and “marshmallow-like.”; “Melty,” “melts in your mouth,” “quickly dissolving,” and “over and done with quickly.”; and easy to chew and swallow, with little to no sticking to the teeth.

These findings demonstrate that the claimed chewable compositions may be classified as Fast Melt compositions. The Fast Melt compositions produce a rapid-dissolution mouthfeel with minimal mastication requirements, a key differentiator from both chalky tablets and traditional chewy-bite antacid products.

### Example 2: Comprehensive Texture Characterization of the Disclosed Chewable Compositions

### Sensory Characterization of Fast Melt Compositions Using Descriptive Analysis (DA)

A sensory texture evaluation was performed to characterize the various compositions. Fast Melt compositions comprise a sugar or sugar-alcohol base combined with one or more hydrocolloids and are designed to disintegrate rapidly in the mouth and disperse easily upon chewing. The primary objectives of the study were to: (i) generate sensory specifications defining the Fast Melt texture profile; (ii) quantitatively measure Fast Melt texture parameters using Descriptive Analysis (DA); and (iii) statistically differentiate Fast Melt compositions from commercial food, supplement, gummy, and over-the-counter (OTC) chewable products.

### Study Design

DA-trained sensory panelists evaluated inventive Fast Melt compositions and multiple commercially available benchmarks including chewable antacids, quick-dissolve tablets, OTC gummies, and confectionery gummies. The study utilized the following DA texture lexicon parameters: rubberiness during chew (15-point scale; target specification 1-3); dissolvability (15-point scale; target specification >10); and toothpull (15-point scale; target specification NMT 2.5).

Panelists were trained according to Spectrum Sensory Methodologies using calibration standards and numeric anchors for texture intensity.

Three Fast Melt compositions were evaluated: Fast Melt 1, Fast Melt 2, and Fast Melt 3. The samples evaluated are listed in Table 2.

TABLE 2

| | | | | 3 |
|---|---|---|---|---|
| | | | Product/ | Digit |
| Form | | Category | API per piece serving | Code |
| Fast melt | 1 | Fast melt | Fast Melt 1 | 406 |
| | 2 | Fast melt | Fast Melt 2 | 670 |
| | 3 | Fast melt | Fast Melt 3 | 342 |
| Alternative | 4 | Chewable Tablet | Rolaids Original Mint: | 543 |
| Forms | | | Calcium Carbonate | |
| | | | 675 mg, Magnesium | |
| | | | Hydroxide 135 mg. | |
| | 5 | RapidMelts | Zicam RapidMelts QDT | 393 |
| | 6 | Chewable Tablet | Tums Chewy Bites- | 296 |
| | | | Antacids-750 mg, | |
| | | | Assorted Berry | |
| | 7 | Soft Chew | Tums Chewy Delights- | 702 |
| | | | 1177 mg, Very Cherry | |
| Gummy | 8 | Pectin | Natures Made Calcium | 134 |
| Supplement | | | Gummies 500 mg | |
| | 9 | Agar | Spring Valley Magnesium | 748 |
| | | | Citrate Gummies-165 mg | |
| | 10 | Pectin | Natures Made Vitamin | 701 |
| | | | C Gummies- 500 mg | |
| | 11 | Sugar free | Natures Made D | 564 |
| | | Pectin/Maltitol | Gummies 50 mcg | |
| | 12 | Pectin and | Nature's Bounty Hair Skin | 188 |
| | | Gelatin | Nails Advanced: Vitamin A | |
| | | | 75 mcg, Vitamin C 9 mg, | |
| | | | Vitamin E 4.05 mg, Biotin | |
| | | | 3000 mcg | |
| | 13 | Gelatin | HEB Omega- | 578 |
| | | | 3 Gummies- 70 mg | |
| | 14 | Sugar | Metamucil Fiber Gummies- | 984 |
| | | free Inulin | 5 g | |
| | | and Pectin | | |
| Candy/Food | 15 | Starch and | Life Savers Gummies, 5 | 635 |
| | | Gelatin | Assorted Flavors | |
| | 16 | Starch | Swedish Fish | 247 |
| | 17 | Soft Panning | Jelly Belly Jelly Beans, | 971 |
| | | | Assorted Flavors | |

Comparators included Rolaids®, Zicam® RapidMelts, TUMS® Chewy Bites, multiple Nature's Made® gummy products, and confectionery gummies such as Life Savers®, Swedish Fish®, and Jelly Belly®.

### Rubberiness Evaluation

Rubberiness of the compositions in Table 2 was quantified using a trained sensory Descriptive Analysis (DA) procedure designed to evaluate the elastic resistance of the product during mastication. For this assessment, “rubberiness” is defined as the amount of resistance felt against the molars when the sample is compressed and released during chew-down, corresponding to the degree of resilience exhibited by the sample. Lower values indicate little to no resilience, while higher values indicate substantial elastic rebound.

### Test Method

Each sample was evaluated according to the following standardized procedure: 1) A single unit of the chewable composition was placed between the molar teeth; 2) The panelist chewed the sample 4-6 times over a period of approximately 3-5 seconds; 3) Beginning with the third chew, the panelist evaluated the perceived elastic resistance; and 4) A final rubberiness score was recorded immediately after the last chew.

All evaluations were conducted by trained sensory panelists operating under synchronized timing and standard palate-cleansing procedures.

### Calibration and Scale Anchors

To ensure consistent scoring across panelists, rubberiness values were calibrated using reference foods assigned to fixed numerical intensities on a 15-point sensory scale. Representative standards are shown in Table 3.

TABLE 3

| Scale | | | Sample | |
|---|---|---|---|---|
| Value | Reference | Details | Size | |
| 1 | Marshmallow | Jet-Puffed, Kraft, regular size | 1 | piece* |
| 2.5 | String cheese | Food Lion Low Moisture Part- | 1 | bite† |
| | | Skim Mozzarella String Cheese | | |
| 3.5-4.5 | Turkey sausage | Jimmy Dean Fully Cooked | 1 | bite‡ |
| | | Turkey Sausage Patty | | |
| 9 | Gummy candy | Lifesavers Gummies, any flavor | 1 | piece |
| 13 | Gummy bears | Haribo Gold, any flavor | 1 | piece |

*Marshmallow reference pieces were bitten with the incisors to obtain a portion appropriately sized for placement between the molars.
†String cheese was served cold, cut into segments approximately 2 cm in length.
‡Turkey sausage bites were prepared in a microwave according to package instructions and served warm.

### Results

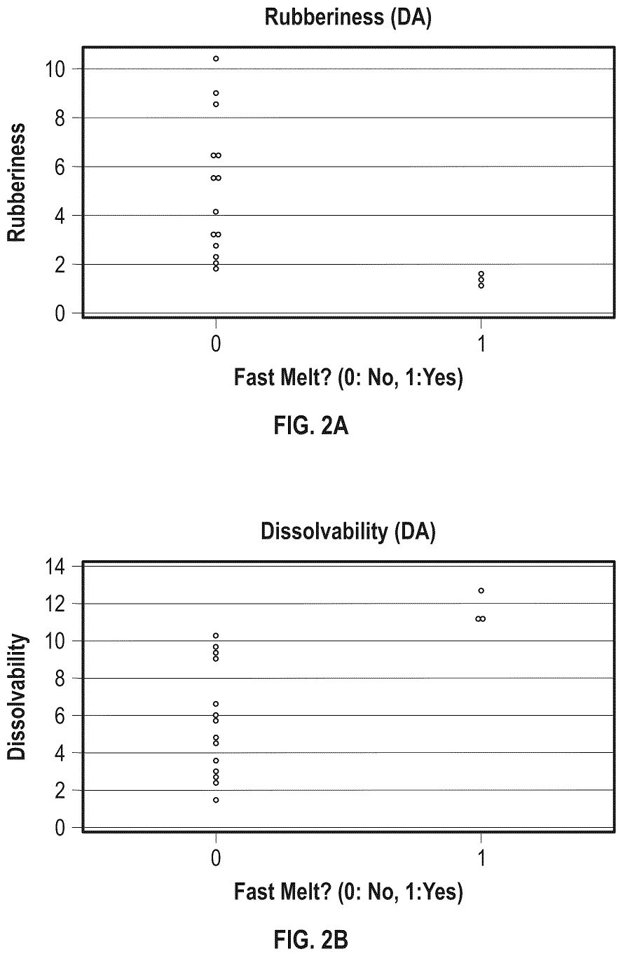

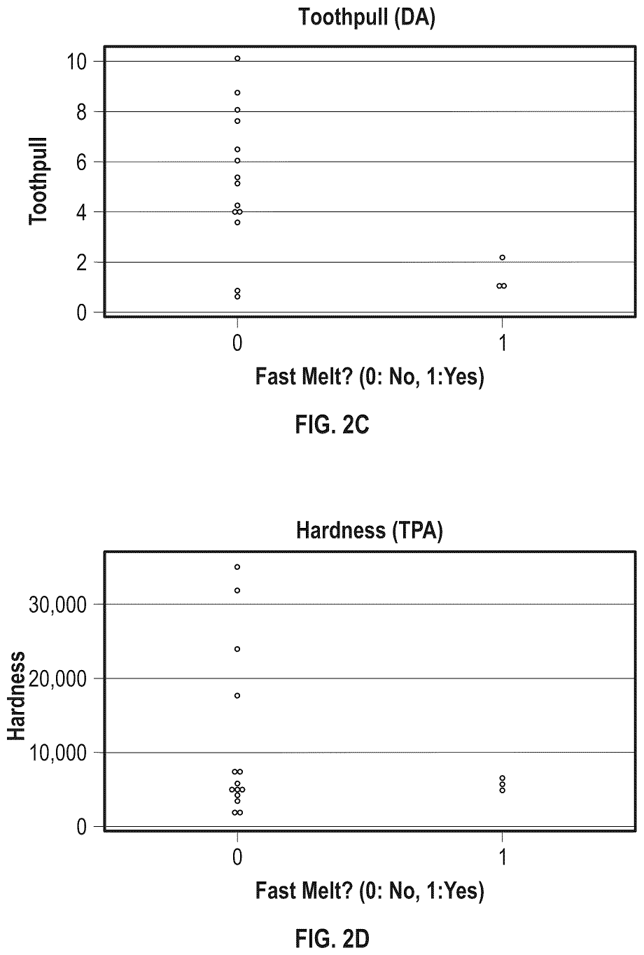

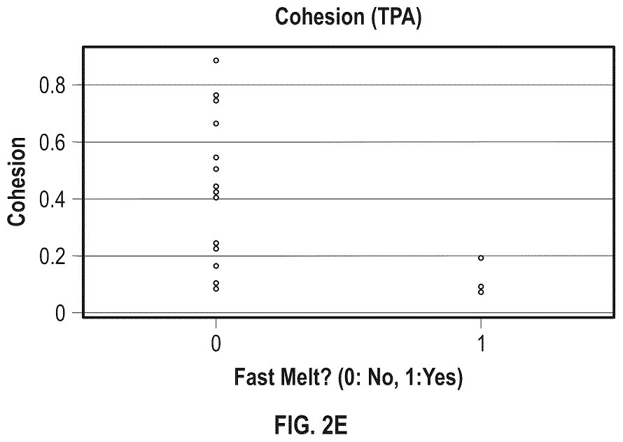

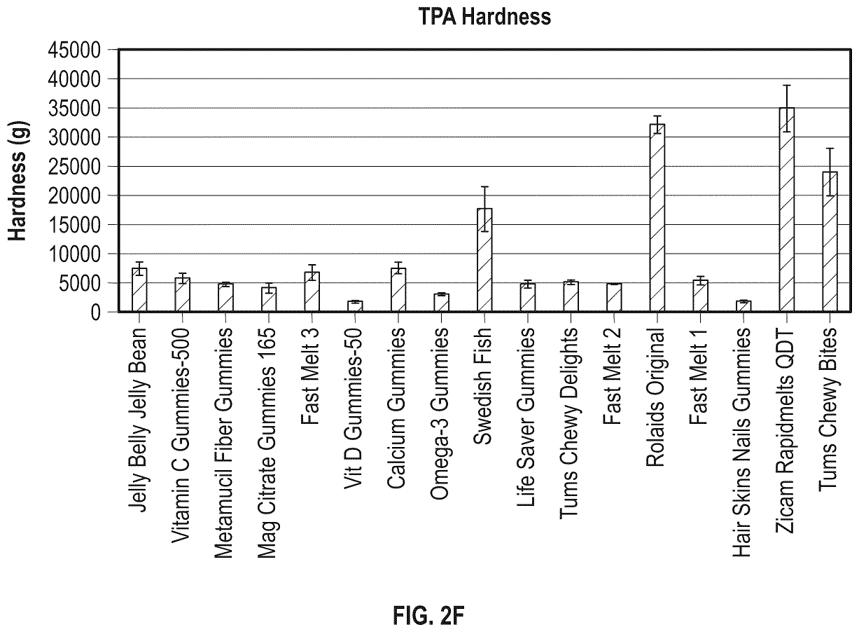

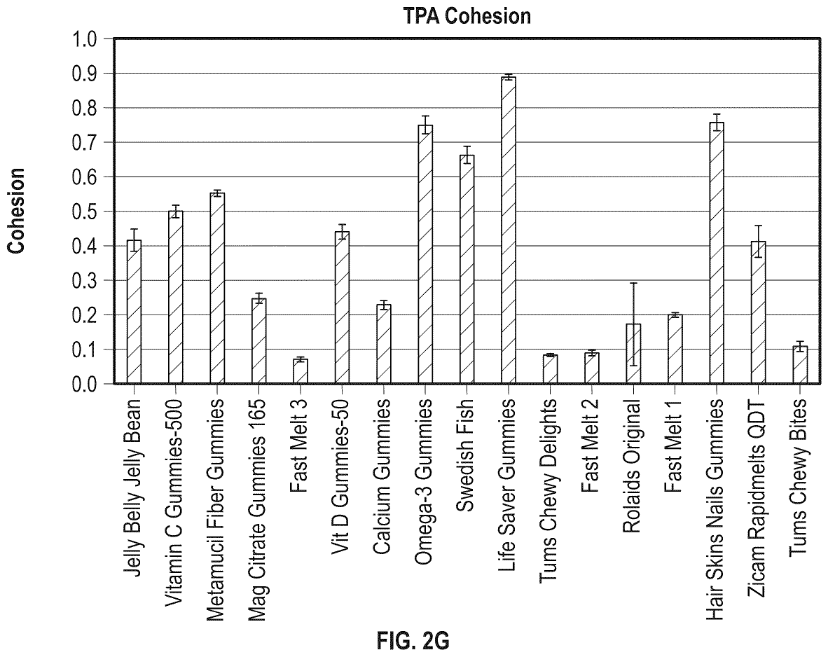

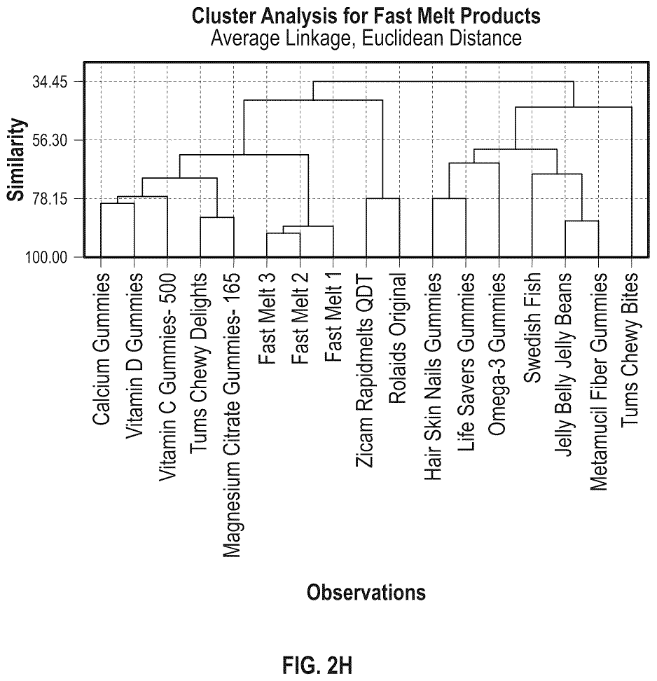

As shown in FIG. 2A and Table 4, Fast Melt compositions exhibited the lowest mean rubberiness among nearly all tested products, indicating minimal resilience and confirming their soft, melt-in-mouth texture relative to conventional gummy candies or gel-based chewable supplements. Fast Melt compositions were significantly lower in rubberiness compared to most commercial gummies. The commercial gummies typically demonstrated substantially higher elasticity.

TABLE 4
Rubberiness

| Name | Fast Melt | Rubberiness |
|---|---|---|
| Fast Melt 2 | Yes | 1.1 |
| Fast Melt 1 | Yes | 1.4 |
| Fast Melt 3 | Yes | 1.5 |
| Vitamin D Gummies | No | 1.8 |
| Calcium Gummies | No | 2.0 |
| Zicam Rapidmelts QDT | No | 2.3 |
| Magnesium Citrate Gummies- 165 | No | 2.8 |
| Vitamin C Gummies- 500 | No | 3.1 |
| Tums Chewy Delights | No | 3.3 |
| Rolaids Original | No | 4.2 |
| Metamucil Fiber Gummies | No | 5.5 |
| Swedish Fish | No | 5.6 |
| Hair Skin Nails Gummies | No | 6.4 |
| Jelly Belly Jelly Beans | No | 6.5 |
| Tums Chewy Bites | No | 8.6 |
| Life Savers Gummies | No | 9.0 |
| Omega-3 Gummies | No | 10.3 |

Despite being low-rubberiness products, Fast Melt compositions retained a slight gummy resilience, consistent with their hydrocolloid matrix. Fast Melt rubberiness values overlapped statistically with: Nature's Made® Vitamin D Gummies (564), Nature's Made® Tricalcium Phosphate Gummies (134), and Zicam® Quick Dissolve Tablets (393).

This demonstrates that Fast Melt compositions deliver minimal chew resistance while retaining a soft, cohesive mouthfeel that differentiates them from conventional gummies.

### Dissolvability Evaluation

Dissolvability of the compositions in Table 2 was evaluated using a trained sensory Descriptive Analysis (DA) method designed to quantify the rate at which a sample breaks down and transitions into a bolus during mastication. For purposes of this assessment, dissolvability is defined as the rate at which the sample dissolves during chew-down, with lower values indicating slow disintegration and higher values indicating rapid breakdown.

### Test Method

Each sample was assessed using the following standardized procedure: 1) A unit of the chewable composition was placed between the molar teeth; 2) The panelist chewed the sample at a consistent rate until the sample transitioned from a formed structure into a bolus or paste-like mass (phase change); and 3) The time and number of chews required for this transformation were used to assign a dissolvability score, with longer transformation times corresponding to lower scores and faster breakdown corresponding to higher scores.

The procedure mirrors comparison calibration using potato chips, in which a sample is chewed until it loses its solid structure and becomes a cohesive mass. All evaluations were conducted in synchronized sessions with palate cleansers provided to maintain panel accuracy.

### Calibration and Scale Anchors

To ensure uniform scoring across panelists, dissolvability was calibrated using reference foods assigned specific numeric intensities on a 15-point sensory scale. Representative standards are shown in Table 5.

TABLE 5

| Scale | | | Sample | |
|---|---|---|---|---|
| Value | Reference | Details | Size | |
| 2 | Wheat cereal | Wheaties, General Mills | 2 | flakes |
| 4 | Baked potato | Baked Lay's Original Potato | 1 | chip |
| | chips | Crisps | | |
| 6 | Cheez Doodles | Wise Cheez Doodles or equivalent | 1 | piece |
| | (crunchy) | | | |
| 10 | Puffed corn | Kix Original | 3 | pieces |
| | cereal | | | |
| 12 | Puffed Cheez | Wise Cheez Doodles Baked Puffs | 1 | piece |
| | Doodles | or equivalent | | |
| 14 | Cotton candy | Any brand | 1 | bite |

Results: As shown in FIG. 2B and Table 6, Fast Melt composition demonstrated the highest dissolvability means among all products tested. Fast Melt compositions dissolved very quickly in the mouth, transitioned to a swallowable bolus with minimal chewing, and significantly outperformed conventional gummies, which showed much lower dissolvability values. Using ANOVA and post-hoc multiple comparison, it was determined that Fast Melt compositions were statistically similar to: Zicam® Quick Dissolve Tablets and Nature's Made® Sugar-Free Pectin Vitamin D Gummies.

TABLE 6
Dissolvability

| Name for Patent | Fast Melt | Dissolvability |
|---|---|---|
| Fast Melt 1 | Yes | 11.3 |
| Fast Melt 2 | Yes | 12.7 |
| Fast Melt 3 | Yes | 11.2 |
| Calcium Gummies | No | 9.0 |
| Hair Skin Nails Gummies | No | 3.5 |
| Swedish Fish | No | 2.4 |
| Tums Chewy Bites | No | 2.6 |
| Zicam Rapidmelts QDT | No | 10.4 |
| Rolaids Original | No | 9.3 |
| Vitamin D Gummies | No | 9.7 |
| Omega-3 Gummies | No | 3.1 |
| Life Savers Gummies | No | 1.5 |
| Vitamin C Gummies- 500 | No | 6.7 |
| Tums Chewy Delights | No | 4.4 |
| Magnesium Citrate Gummies- 165 | No | 6.0 |
| Jelly Belly Jelly Beans | No | 4.7 |
| Metamucil Fiber Gummies | No | 5.6 |

This performance confirmed that Fast Melt compositions achieve the intended rapid oral disintegration, supporting their classification as Fast Melt hydrocolloid dosage forms. These results confirm the Fast Melting properties of the compositions and distinguish them from conventional gummies, chewy gels, and traditional chewable tablets, which require more extensive mastication before breakdown.

### Toothpull Evaluation

To quantify the adhesion characteristics of the chewable compositions described herein, toothpull was evaluated using a trained sensory Descriptive Analysis (DA) procedure. For purposes of this method, toothpull is defined as the force required to separate the upper and lower teeth during chew-down, corresponding to the degree of stickiness or adhesion exhibited by the sample. Lower values indicate little to no sticking between the teeth, whereas higher values reflect substantial resistance due to adhesive mass formation.

### Test Method

Toothpull was assessed according to the following standardized procedure: 1) A single unit of the chewable composition was placed between the molar teeth; 2) The panelist chewed the sample five times within approximately 10 seconds, ensuring consistent timing across evaluations; 3) During these chews, the panelist evaluated the force required to pull the teeth apart when the sample compressed and stretched between the occlusal surfaces; and 4) A final toothpull score was recorded immediately after the chew sequence.

All testing sessions were synchronized across panelists, and palate cleansers were provided to prevent carryover effects between samples.

### Calibration and Scale Anchors

Panelists calibrated their evaluations against a set of reference materials assigned fixed numeric values on a 15-point toothpull intensity scale. Representative calibration standards are shown in Table 7.

TABLE 7

| Scale | | | Sample | |
|---|---|---|---|---|
| Value | Reference | Details | Size | |
| 1 | Granola bar | Quaker ® Chewy Granola Bars | ⅓ | bar |
| | | (Dark Chocolate Chunk) | | |
| 4 | Gummy candy | Lifesavers Gummies | 1 | piece |
| 10 | Gummy candy | Swedish Fish (original) | 1 | piece |
| 14 | Gummy candy | JujyFruits (any flavor) | 1 | piece |

These reference values anchor the sensory scale from low adhesion (granola bar) to moderate adhesion (Lifesavers), high adhesion (Swedish Fish), and very high adhesion (JujyFruits). Panelists used these anchors to ensure reproducible scoring across evaluations.

### Results

As shown in FIG. 2C and Table 8, all commercial gummy comparators displayed significantly higher toothpull than 2.5. Fast Melt compositions exhibited very low toothpull, with mean values between 1 and 2. This indicates minimal adhesion and little to no force required to separate the teeth during chewing. This low toothpull performance distinguishes the Fast Melt compositions from typical pectin, gelatin, and starch-based gummy products, which exhibit substantially higher toothpull values under identical testing conditions. This is a noticeable advantage compared to typical gummy and chewable products. Fast Melt compositions overlapped with Zicam® Quick Dissolve Tablet, Rolaids® Original Chewable Tablets, and Nature's Made® Pectin Vitamin C. Only hard chewable tablets showed lower toothpull values, but those products were significantly firmer than Fast Melt compositions.

TABLE 8
Toothpull

| Name for Patent | Fast Melt | Toothpull |
|---|---|---|
| Rolaids Original | No | 0.6 |
| Zicam Rapidmelts QDT | No | 0.8 |
| Fast Melt 2 | Yes | 1.0 |
| Fast Melt 3 | Yes | 1.1 |
| Fast Melt 1 | Yes | 2.1 |
| Vitamin C Gummies- 500 | No | 3.6 |
| Calcium Gummies | No | 3.9 |
| Life Savers Gummies | No | 4.0 |
| Hair Skin Nails Gummies | No | 4.3 |
| Tums Chewy Bites | No | 5.2 |
| Vitamin D Gummies | No | 5.3 |
| Tums Chewy Delights | No | 5.9 |
| Magnesium Citrate Gummies- 165 | No | 6.6 |
| Metamucil Fiber Gummies | No | 7.6 |
| Jelly Belly Jelly Beans | No | 8.1 |
| Omega-3 Gummies | No | 8.7 |
| Swedish Fish | No | 10.0 |

These results show a unique combination of low adhesion and rapid breakdown, distinguishing Fast Melt compositions from standard gummies, chewables, and soft-chew tablets.

### Sensory-Driven Differentiation of Fast Melt Compositions

Across all measured parameters, Fast Melt compositions demonstrated a distinct and statistically separable texture profile: 1) low rubberiness (1-3 range): substantially lower elasticity than most gummy products and slight gummy resilience preserved for consumer familiarity; 2) high dissolvability (>10): fast, efficient mouth breakdown, rapid transition to bolus mass, and distinct from traditional gummies and chewable tablets; and 3) low toothpull (<2.5): non-sticky chew, minimal residue on teeth, and advantageous versus commercial gummies and soft chews.

These results collectively confirm that Fast Melt compositions offer a unique combination of rapid disintegration, low chew resistance, and low adhesion, properties not found together in conventional gummies, chewable tablets, or typical quick-dissolve formats.

### Measurement of Hardness by Texture Profile Analysis (TPA)

Hardness of the compositions in Table 2 was quantified using a Texture Profile Analysis (TPA) method designed to simulate the initial biting action applied to the product during oral consumption. For purposes of this testing method, hardness is defined as the maximum peak force required to compress the sample during the first compression cycle, reflecting the initial firmness and resistance to deformation of the chewable composition.

### Instrumentation and Setup

Hardness measurements were performed using a TA.XT Plus Texture Analyzer (Stable Micro Systems) equipped with a TA-4 acrylic disk probe, a 50-pound load cell, and a TA-90 metal platform for sample placement.

The sample was positioned on the platform, and the probe was programmed to compress the sample to 50% strain, generating a force-time curve. The maximum force recorded during the first compression was identified as the hardness value. The probe and platform were cleaned with alcohol between tests to prevent sample residue interference.

### Test Procedure

A single chewable unit was placed at the center of the TA-90 platform. The TA-4 probe compressed the sample to 50% deformation at a controlled speed. The analyzer recorded a force-time curve for the duration of the test.

Hardness was defined as the maximum peak force during the first compression cycle.

Additional TPA parameters (cohesiveness, adhesiveness, resilience, springiness, chewiness) were calculated but hardness was used as the primary metric for differentiating fast-melt products.

Replicate testing was performed for each sample, and mean values were calculated for comparison across product types.

### Calibration and Interpretation

In TPA methodology, hardness is not calibrated to sensory scale anchors but is instead objectively reported in grams of force (g). Hardness values are typically: very high for compressed chewable tablets, moderate-high for dense gummy confections, low-moderate for certain pectin or mixed-gel gummies. Hardness values are relatively high for Fast Melts.

### Results

As seen in FIGS. 2D and 2F and Table 9, in Fast Melt compositions, hardness values averaged below 10,000 grams. These values contrasted sharply with the higher hardness observed for comparative products such as Rolaids chewable tablets and Zicam RapidMelts, while partially overlapping with certain pectin or agar gummies, which exhibited similar cohesiveness but distinct differences in dissolvability and rubberiness.

TABLE 9
Hardness

| Name for Patent | Fast Melt | Hardness |
|---|---|---|
| Zicam Rapidmelts QDT | No | 34927 |
| Rolaids Original | No | 32147 |
| Tums Chewy Bites | No | 24011 |
| Swedish Fish | No | 17661 |
| Calcium Gummies | No | 7557 |
| Jelly Belly Jelly Beans | No | 7425 |
| Fast Melt 3 | Yes | 6749 |
| Vitamin C Gummies- 500 | No | 5759 |
| Fast Melt 1 | Yes | 5367 |
| Tums Chewy Delights | No | 5083 |
| Fast Melt 2 | Yes | 4802 |
| Metamucil Fiber Gummies | No | 4763 |
| Life Savers Gummies | No | 4747 |
| Magnesium Citrate Gummies- 165 | No | 4087 |
| Omega-3 Gummies | No | 3024 |
| Hair Skin Nails Gummies | No | 1772 |
| Vitamin D Gummies | No | 1740 |

### Measurement of Cohesiveness by Texture Profile Analysis (TPA)

The cohesiveness of the compositions described in Table 2 was evaluated using a Texture Profile Analysis (TPA) method designed to quantify the internal structural integrity of the sample when subjected to repeated deformation. For purposes of this analysis, cohesiveness is defined as the ratio of the positive force area generated during the second compression cycle to the positive force area generated during the first compression cycle, reflecting the extent to which the sample retains its structure after an initial deformation.

### Instrumentation and Testing Setup

Cohesiveness measurements were performed using the same instrument set up at the hardness measurement. The analyzer was programmed to conduct a double-compression test, which simulates two sequential bite cycles. During each test, the probe compressed the sample to 50% strain, generating a force-time curve from which the necessary areas under the curve were calculated. The probe and platform were cleaned between samples to ensure measurement accuracy.

### Test Procedure

A single chewable unit was placed on the TA-90 platform. The TA-4 probe compressed the product to 50% of its original height at a fixed crosshead speed, generating the first compression curve. The probe then returned to its starting position and immediately applied a second compression, generating the second compression curve. The Texture Analyzer software calculated the positive force area under the first compression curve and the positive force area under the second compression curve.

Cohesiveness was calculated as: Cohesiveness=(Area2/Area1) where Area2 is the positive force area of the second compression and Area1 is that of the first compression. The test was repeated ten total times for each sample, and mean values were reported.

### Results

Cohesiveness values range from 0 to 1, with lower values indicating a product that breaks down easily after the first compression (i.e., low structural integrity), and higher values indicating a more resilient, gummy-like structure.

As seen in FIGS. 2E and 2G and Table 10, Fast Melt products exhibit cohesiveness values between 0.05 and 0.3, distinguishing them from conventional gummies, which typically exhibit significantly higher cohesiveness due to stronger gel networks. Across multiple test samples, including Fast Melt 1, Fast Melt 2, and Fast Melt 3, measured cohesiveness values were consistently within the range of 0.05-0.2, confirming that the compositions rapidly break down upon repeated deformation and do not exhibit the structural resilience associated with pectin-, gelatin-, or agar-based gummy products. Some market gummies exhibited similar hardness values but significantly higher cohesiveness, underscoring the utility of cohesiveness as a differentiating parameter.

TABLE 10
Cohesion

| Name for Patent | Fast Melt | Cohesion |
|---|---|---|
| Fast Melt 3 | Yes | 0.07 |
| Tums Chewy Delights | No | 0.08 |
| Fast Melt 2 | Yes | 0.09 |
| Tums Chewy Bites | No | 0.11 |
| Rolaids Original | No | 0.17 |
| Fast Melt 1 | Yes | 0.20 |
| Calcium Gummies | No | 0.23 |
| Magnesium Citrate | No | 0.25 |
| Gummies- 165 | | |
| Zicam Rapidmelts QDT | No | 0.41 |
| Jelly Belly Jelly Beans | No | 0.42 |
| Vitamin D Gummies | No | 0.44 |
| Vitamin C Gummies- 500 | No | 0.50 |
| Metamucil Fiber Gummies | No | 0.55 |
| Swedish Fish | No | 0.66 |
| Omega-3 Gummies | No | 0.75 |
| Hair Skin Nails Gummies | No | 0.76 |
| Life Savers Gummies | No | 0.89 |

FIG. 2H is a graph representing a hierarchical cluster analysis of the data collected in Example 2.

The dendrogram shows that the Fast Melt compositions (Fast Melt 1, Fast Melt 2, and Fast Melt 3) cluster tightly together, indicating that they share a high degree of similarity in the measured texture parameters (dissolvability, rubberiness, toothpull, and cohesiveness). These Fast Melt compositions form a distinct branch separate from most confectionery gummies (e.g., Calcium Gummies, Vitamin D Gummies, Vitamin C Gummies, Hair Skin Nails Gummies, Omega-3 Gummies) and from sticky or elastic candy-type products such as Swedish Fish and Jelly Belly Jelly Beans. This separation reflects the substantially different texture profile of fast-melt products compared to typical gummy compositions.

The dendrogram also shows that Fast Melt compositions are more similar to each other and to quick-dissolve or soft chew products like Zicam RapidMelts ODT and Rolaids Original chewable tablets, than they are to traditional gummy confections. Certain products such as Magnesium Citrate Gummies and Tums Chewy Delights form intermediate clusters. This indicates partial similarity but not identical texture characteristics to Fast Melt products.

Overall, the cluster analysis visually demonstrates that Fast Melt compositions form their own unique texture category, clearly separated from standard gummy supplements, jelly confections, and many chewable antacid forms. This supports the conclusion that Fast Melt compositions possess distinguishing structural and sensory characteristics that differentiate them from other commercially available chewable or gummy products.

### Example 3: Texture and Stability Development of Fast Melt 1 Over Three Months

A 3-gram Fast Melt 1 composition (Table 11) was prepared to evaluate the evolution of texture, water activity, and disintegration properties over a three-month period. The objective of this study was to characterize how Fast Melt gels develop their characteristic texture over time and to determine the time point at which the product meets the proposed Fast Melt specifications for hardness and cohesiveness. Testing was performed at predefined intervals, including Time 0, Day 2, Week 1, Month 1, Month 2, and Month 3.

TABLE 11

| | Acceptable range (% by weight of total |
|---|---|
| Ingredient Name | composition) |
| Water | 13-20% |
| Sugar | 45-65% |
| Corn Syrup | 10-25% |
| Glycerin | 2-10% |
| Taste masking | 0-10% |
| Carrageenan | 0.5-2.5% |
| Emulsifier | 0-1% |
| Insoluble Solids | 0-15% |
| Flavor | 0-5% |
| Active Pharmaceutical | 0% |
| Ingredients | |
| Color | 0-1% |
| Coating | 0-10% |

### Water Activity Assessment

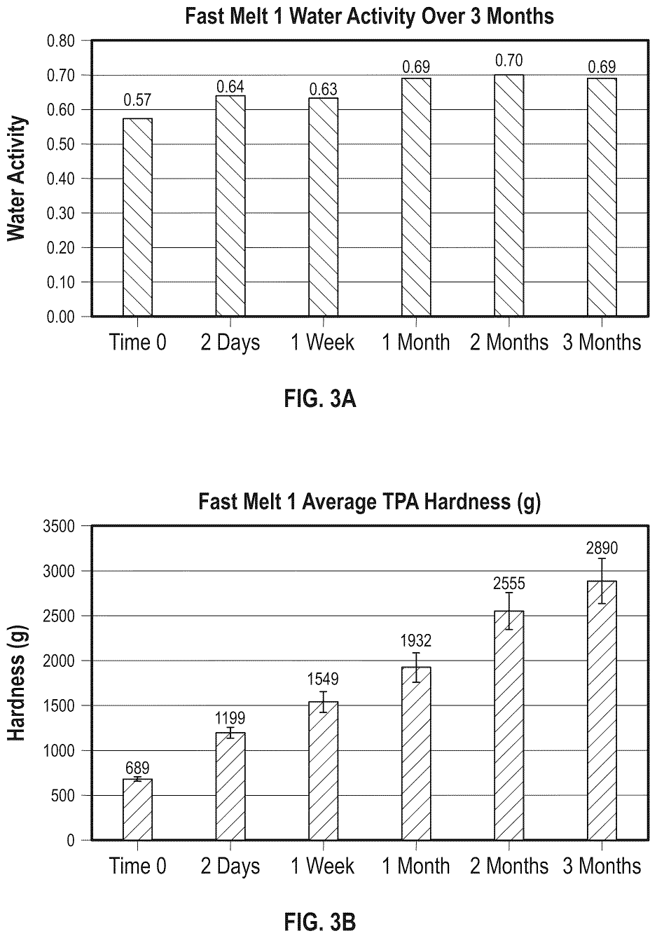

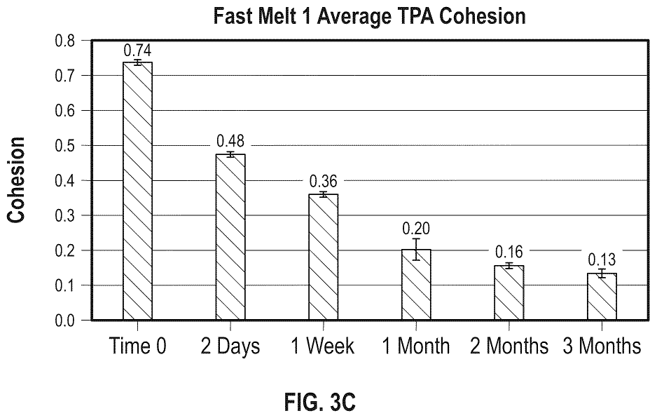

Water activity was measured at each time point to help characterize product safety and to assess changes related to crystallization and moisture migration. As shown in FIG. 3A, at the one-month time point, the water activity remained below the target of 0.8 established for high-pH gel systems. Continued monitoring confirmed that the water activity remained within acceptable ranges throughout the study.

### Texture Profile Analysis (TPA)

### Hardness

Hardness was measured using Texture Profile Analysis (TPA) with a double-compression method. As shown in FIG. 3B, the hardness of the Fast Melt composition continued to increase over the first month, indicating ongoing surface crystallization and structural development. Despite this increase, the hardness remained below 10,000 g, placing the material within the Fast Melt hardness range specified for the invention. This demonstrates that the product maintains the firm exterior required for handling while preserving a soft internal matrix conducive to fast melt behavior.

### Cohesiveness

Cohesiveness values decreased steadily during the first month. As shown in FIG. 3C, by month 1, cohesiveness had dropped below 0.3, meeting the proposed Fast Melt specification of 0.05-0.3. This decline in cohesiveness reflects the progressive weakening of internal structural bonds, which facilitates rapid breakdown during mastication and contributes to the soft, collapsible texture characteristic of Fast Melt compositions.

### Disintegration Testing

Disintegration was evaluated at month 1 using USP<701> methodology with purified water as the test solvent with pieces being cut in half. The 3-gram Fast Melt dissolved in 18 minutes and 20 seconds, demonstrating a faster disintegrating profile relative to standard gummy formulations. This performance indicates that the Fast Melt gel base is suitable for use in OTC drug actives requiring dissolution or disintegration within compendial specifications.

### Overall Texture Development

Over the course of three months, the Fast Melt exhibited: increasing hardness (but consistently <10,000 g), decreasing cohesiveness (≤0.3 by month 1), stable water activity below the threshold (≤0.8), and acceptable disintegration performance suitable for Fast Melt applications.

Collectively, these findings confirm that Fast Melt gels undergo a maturation process that yields their characteristic texture approximately one month after manufacture. The resulting product meets the defined Fast Melt criteria for hardness, cohesiveness, and functional disintegration.

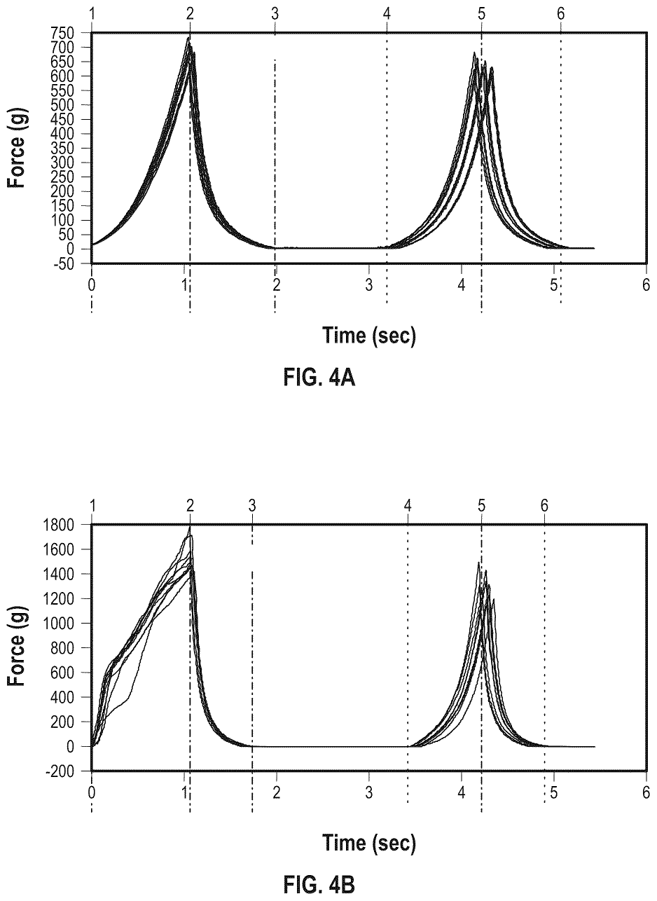

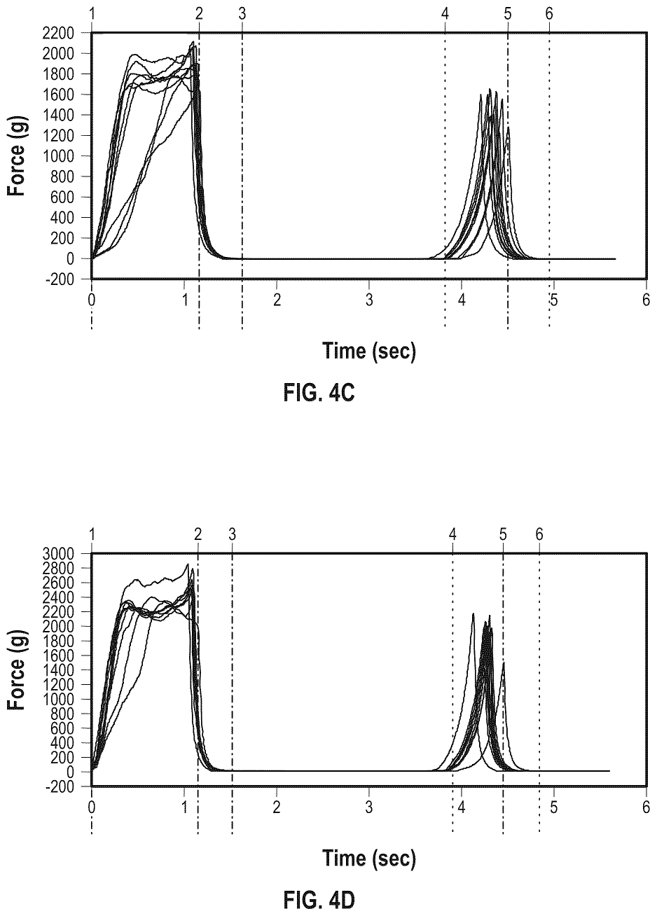

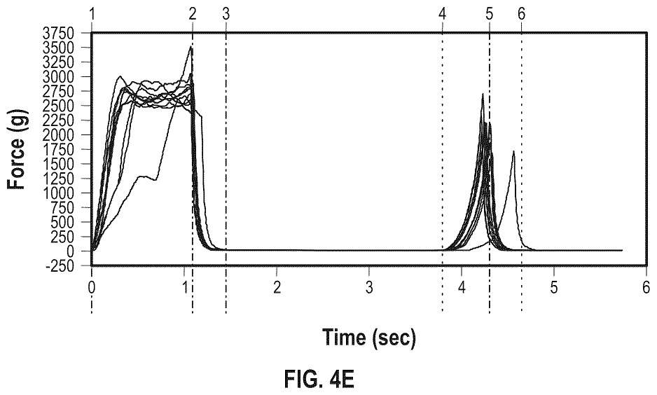

The laboratory-produced 3-gram Fast Melt 1 sample was evaluated to characterize the evolution of its mechanical texture profile over a three-month period. Texture Profile Analysis (TPA) was performed at time 0 (T0), week 1 (W1), month 1 (M1), month 2 (M2), and month 3 (M3). Each evaluation generated force-time compression curves reflecting hardness, fracture behavior, graining development, and changes in cohesiveness and resilience. Representative TPA curves for each time point are shown in FIGS. 4A-4E.

### T0: Initial Gel-Like Texture

As shown in FIG. 4A, at time 0, the Fast Melt sample exhibited texture curves characteristic of newly formed hydrocolloid gels. The force-time curves displayed symmetrical peaks of similar shape and height across replicates, indicating minimal graining, high cohesiveness, high elasticity/resilience, and low fracture incidence. The lack of fracture peaks demonstrated that, immediately after manufacture, the matrix behaved as a uniform viscoelastic gel with limited crystalline structure development.

### W1: Early Development of Fracture Points

As shown in FIG. 4B, by week 1, the TPA curves began to diverge, and the first signs of structural evolution were observed. The curves showed the appearance of fracture points within the first compression cycle, a slight reduction in resilience, evidenced by an increased area of the first half of the first compression cycle compared to the area of the second half of the first compression cycle, and increased peak hardness. These changes indicate the beginning of controlled crystallization in the outer phase of the sample and partial weakening of internal gel structure.

### M1: Formation of Pronounced Fracture Behavior and Graining

As shown in FIG. 4C, at month 1, the mechanical behavior of the Fast Melt composition had transitioned significantly. The TPA curves displayed pronounced fracture points during the first compression, a substantial decrease in cohesiveness, indicated by minimal recovery during the second compression, and an increase in the peak hardness. These features reflect the formation of a partially crystalline matrix that fractures easily upon biting, which is characteristic of Fast Melt texture.

### M2: Stabilization of Hardness and Continued Reduction in Cohesiveness

As shown in FIG. 4D, by month 2, the fracture-peak signatures became sharper and more consistent across replicates. The curves demonstrated stabilized hardness values, sustained low cohesiveness (≤0.3), a mature grained structure, and persistent fragmentation behavior during compression. The sample maintained Fast Melt characteristics while showing improved sample texture variability.

### M3: Mature Fast Melt Texture

As shown in FIG. 4E, at month 3, the force-time curves indicated a fully matured Fast Melt profile. Key features included: distinct and repeatable fracture peaks, significantly reduced cohesiveness compared to T0, stable hardness values within the Fast Melt specification, and well-developed graining across the entire outer phase of the sample.

These results confirm that the Fast Melt texture fully develops after aging for approximately 2-4 weeks, with continued refinement through month 3.

### CONCLUSION

This example demonstrated that Fast Melt chewable gels undergo a predictable transformation from an initially cohesive gel (T0) to a brittle, grained, rapidly fracturing structure (M1-M3). The evolution of texture, confirmed by increased fracture-peak visibility, reduced cohesiveness, and increased peak hardness, correlated with the formation of the patented Fast Melt texture profile.

### Rheological Characterization of Fast Melt Compositions

Rheological measurements were performed using the Anton Paar MCR-92 Compact Rheometer. Rheology provides a combined assessment of viscous and elastic (solid-like) responses. This helps to distinguish true Fast Melt textures from gummy-type viscoelastic liquids.

A diverse set of commercial and inventive products was selected to evaluate rheological differences between Fast Melt compositions and conventional gummy systems. Table 12 lists the samples and their corresponding hydrocolloid systems. The comparators included starch-based gummies, gelatin-based gummies, pectin gels, agar gels, and carrageenan-based Fast Melt compositions. This range of hydrocolloid matrices enabled direct comparison of viscoelastic properties, thixotropic recovery, and sensory dissolvability across distinct gel structures.

TABLE 12

| # | Sample Name | Hydrocolloid |
|---|---|---|
| 1 | Lifesavers Gummies, any flavor | Starch/Gelatin |
| 2 | Swedish Fish, original | Starch |
| 3 | JujyFruits Candy, any flavor | Starch |
| 4 | Annie's Bunny Fruit Flavored Snacks | Pectin |
| 5 | Spring Valley Adult Gummy 250 mg Magnesium | Agar |
| 6 | Spring Valley Adult Gummy 250 mg Vitamin C | Pectin |
| 7 | HEB 70 mg Omega-3 | Gelatin |
| 8 | Haribo Gummy Bears | Gelatin |
| 9 | Fast Melt 4 | Carrageenan |
| 10 | Fast Melt 2 | Carrageenan |
| 11 | Non-Fast Melt 1 | Carrageenan |
| 12 | Non-Fast Melt 2 | Carrageenan |
| 13 | Nature Made Calcium 500 mg | Pectin |

### Amplitude Sweep Testing

Amplitude sweep testing is a rheological method in which a sample is subjected to oscillatory deformation while the strain amplitude is gradually increased at a constant oscillation frequency. During this test, the instrument measures two key moduli: storage modulus (G′) the elastic or “solid-like” component; and loss modulus (G″) the viscous or “liquid-like” component. The purpose of the test is to determine the Linear Viscoelastic Range (LVER) which is the region where the material's structure remains intact and both G′ and G″ remain constant. As strain increases beyond the LVER, the material's structure begins to collapse, causing G′ and G″ to decrease or cross over.

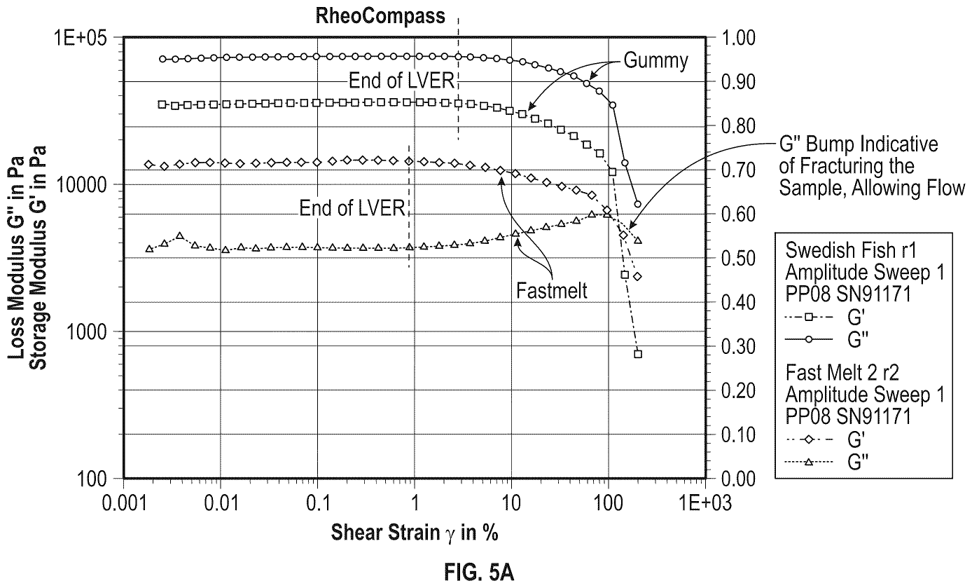

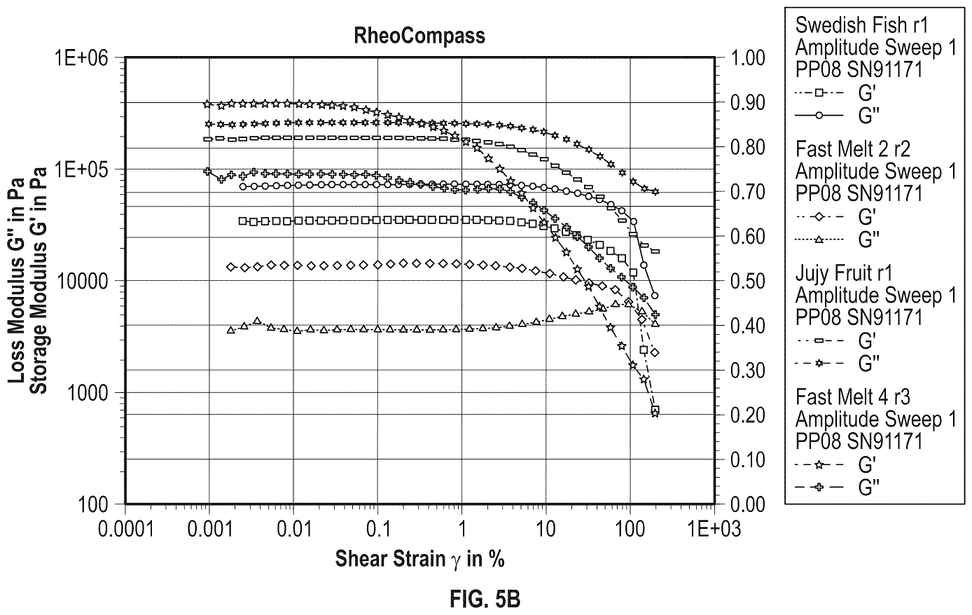

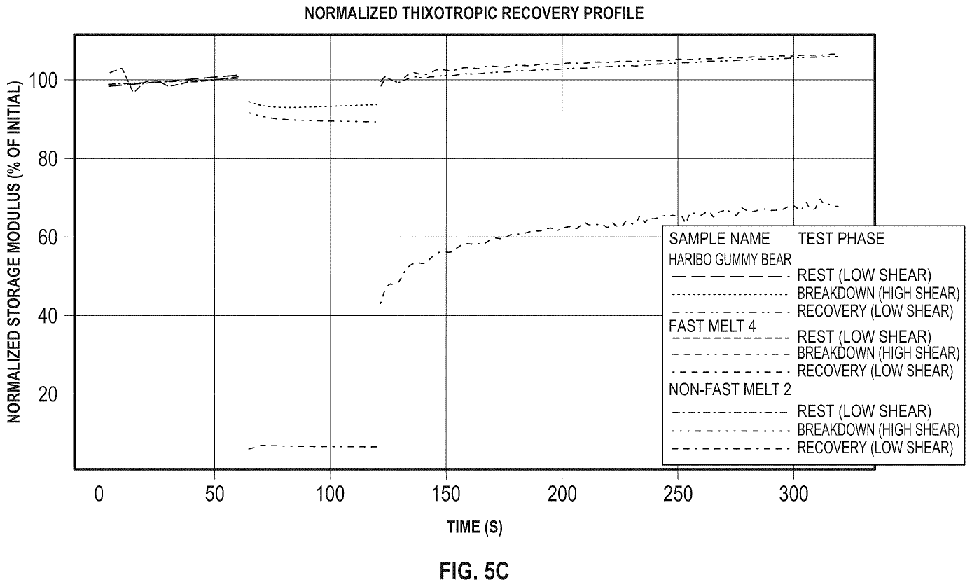

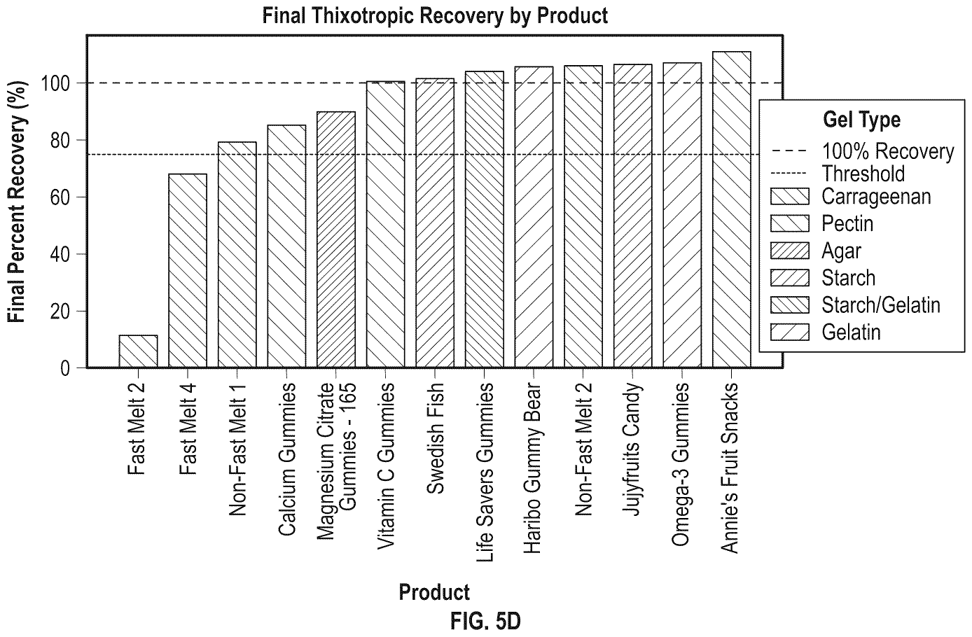

Amplitude sweeps (0.001-200% strain, 10 rad/s, 20° C.) were conducted to determine each product's Linear Viscoelastic Range (LVER). As seen in FIGS. 5A and 5B, Fast Melt compositions showed a solid-dominant profile (G′>G″) within the LVER and rapid structure breakdown once strain exceeded the LVER. This behavior mirrors the amplitude-sweep profiles, where Fast Melt products cluster in the “solid-dominant” region and show earlier structural breakdown than starch- or gelatin-based gummies. Thixotropy is a time-dependent reversible reduction in structure or modulus under shear, followed by partial or complete structure recovery once the shear is removed. Thixotropy is a time-dependent rheological property in which a material becomes less viscous or less structured when subjected to shear (such as stirring, chewing, or mechanical stress) and then gradually rebuilds its structure when the shear is removed. Thixotropy describes a material's time-dependent structural breakdown under shear and its ability to rebuild that structure once the shear is removed. Percent recovery is the quantitative measurement used to evaluate the rebuilding process.

Thixotropy was evaluated using the three-interval test (10 rad/s; low-high-low amplitude): interval 1: Low amplitude (within LVER) measures structure at rest; interval 2: high amplitude (10% strain) disrupts structure; and interval 3: low amplitude—measures recovery.

As shown in FIG. 5C, Fast Melt compositions exhibited low structural recovery (~200 s endpoint), whereas gummies displayed near-complete recovery by the end of Interval 3. This separation is clearly shown in the plot labeled “Normalized Thixotropic Recovery Profile,” where Fast Melt samples maintain significantly lower percent recovery curves than gummy comparators (Haribo, Non-Fast Melt 2)

As seen in FIG. 5D, final percent recovery for each product demonstrated separation between Fast Melt compositions (<~75% recovery) and gummy systems (>~75% recovery), independent of gel agent type. This metric reliably distinguishes true Fast Melt compositions from Non-Fast Melt compositions.

### Example 4: Effects of Ratio of Non-Reducing Sugars to Reducing Sugars, Percent of Insoluble Solids, and Time on Fast Melt Texture and Water Activity

A study was conducted to evaluate how the ratio of non-reducing sugars to reducing sugars, percent of insoluble solids (e.g. calcium carbonate), average molecular weight of reducing sugars used (represented in the study as Dextrose Equivalence), and time affect the water-activity and texture of Fast Melt chewable compositions. The compositions tested are provided in Table 13. A full-factorial design of experiments (DOE) was conducted to systematically characterize how these variables influence hardness, cohesiveness, water activity, and graining over a three-month aging period.

TABLE 13

| | Ranges (% by weight of |
|---|---|
| Ingredient Name | composition) |
| Water | 10%-25% |
| Sugar | 20%-60% |
| Corn Syrup | 15%-50% |
| Carrageenan | 0.5%-4% |
| Emulsifier | 0%-2% |
| Oil | 0%-6% |
| Insoluble Solids | 0%-20% |
| Flavor | 0%-1% |
| Active Pharmaceutical | 0%-20% |
| Ingredients | |
| Color | 0%-1% |
| Coating | 2%-5% |

### Study Design and Experimental Factors

Fast Melt compositions tested had the composition listed in Table 13 were made using a confectionery gel base comprising non-reducing sugar (sucrose), reducing sugars (corn syrup of differing average molecular weight), and carrageenan. The DOE incorporated the following independent variables: non-reducing to reducing sugar ratio in the syrup phase, ranging from 40:60 to 70:30, average chain length of corn syrup used, ranging from approximately 43 DE to approximately 63 DE, and calcium carbonate levels from 0-6%. Testing was conducted over time at time 0, 1 month, 2 months, and 3 months. Controlled variables included carrageenan composition, moisture, emulsifier and oil composition, fixed API concentration for other active insoluble solids present in the formulations, and flavor system.

The responses measured included: water activity (Aw), hardness, cohesiveness, graining thickness, and inner-phase solids content.

### Hardness

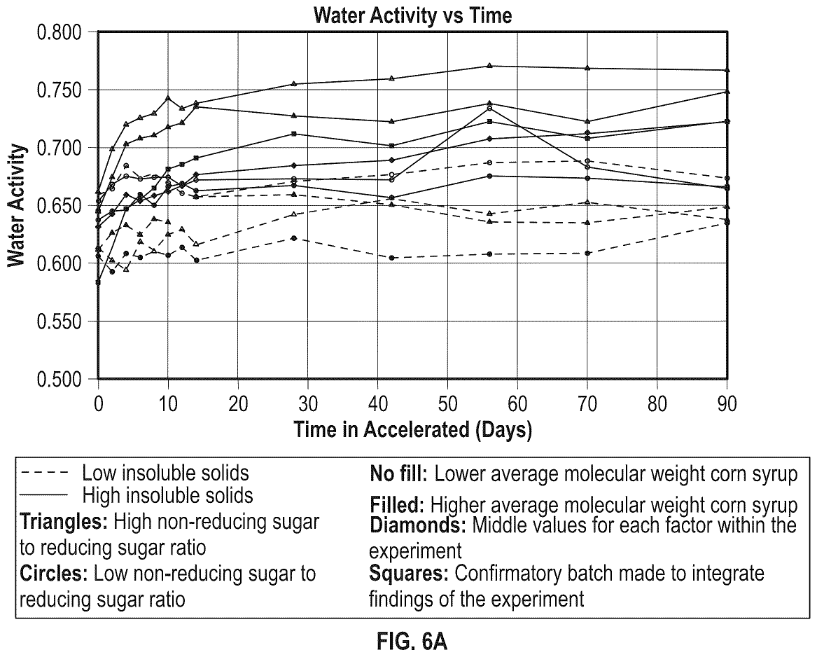

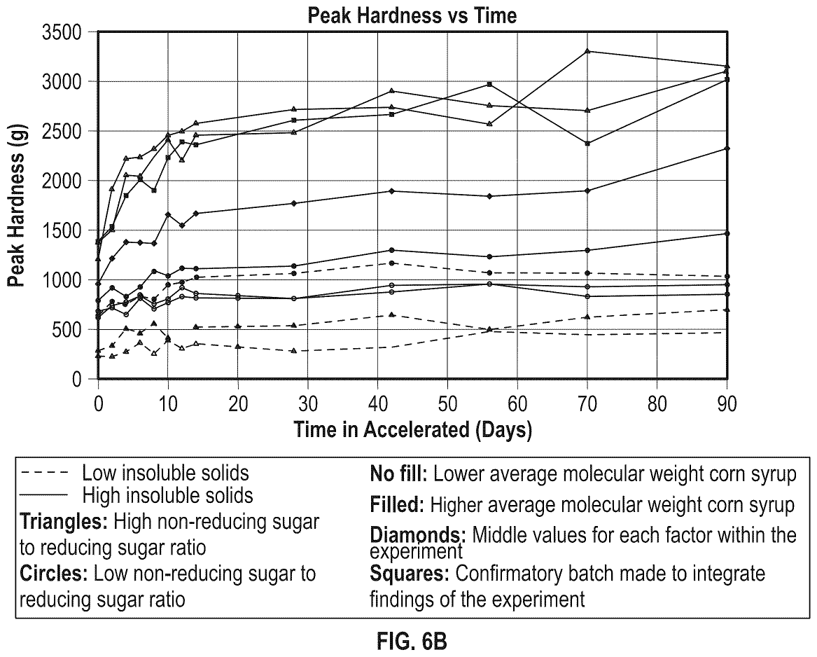

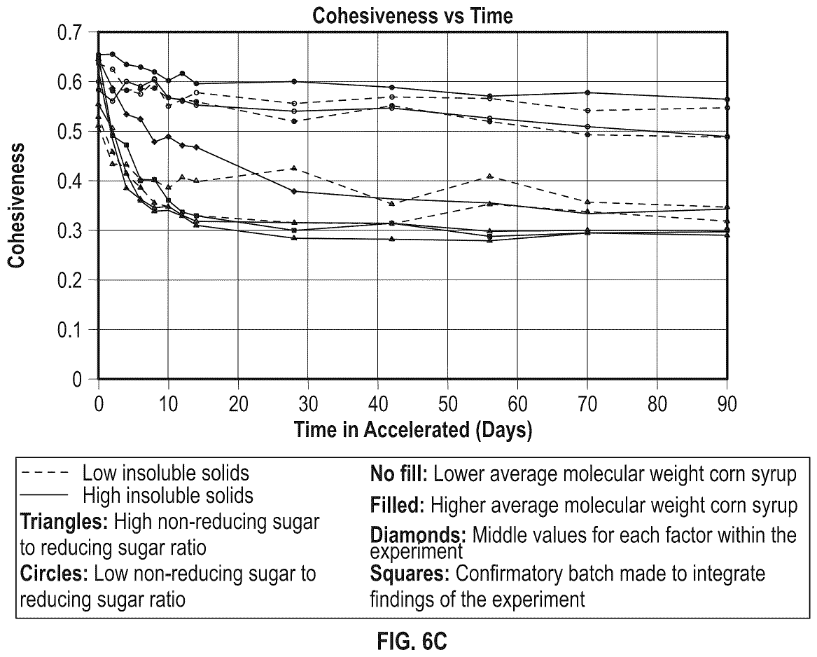

As shown in FIG. 6B, plotted peak hardness values showed that calcium carbonate is a critical lever for achieving the firmness needed for commercial handling. Formulas containing calcium carbonate (solid-line conditions) exhibited significantly higher hardness than formulas without insoluble solids (dashed-line conditions). The highest peak hardness values were observed in formulas combining 6% calcium carbonate with high sucrose ratios (70:30) in the syrup phase. These combinations produced textures firm enough to withstand bottling, transport, and shelf storage without smearing or deformation. Even in these firmer samples, hardness remained within the Fast Melt classification threshold (<10,000 g).

### Cohesiveness

As shown in FIG. 6C, cohesiveness was inversely related to sugar ratio and calcium carbonate level. Low cohesiveness, characteristic of Fast Melt compositions, was observed in samples containing high sugar ratios and 6% calcium carbonate, represented by solid-line square-symbol formulations. These formulations reached cohesiveness values at or below 0.3, meeting the proposed Fast Melt specification. Lower sugar ratios or absence of calcium carbonate resulted in higher cohesiveness, more typical of gummy or gel structures rather than Fast Melt compositions.

This demonstrates that high sucrose ratios combined with insoluble solids promote the formation of textures that do not retain their structure when deformed, which is a key driver of Fast Melt texture.

### Water Activity (Aw)

As shown in FIG. 6A, water activity was strongly influenced by the insoluble solid and syrup composition levers. The lowest Aw values were observed in samples without calcium carbonate. Samples containing calcium carbonate exhibited higher Aw. Syrups containing DE43 corn syrup produced higher Aw values than those formulated with DE63 corn syrup. Increasing total sugar in the syrup phase also elevated Aw.

These results suggest that, in formulations including insoluble solids such as calcium carbonate, selection of carbohydrate components, including DE63 corn syrup and total sugar levels, can influence water activity (Aw) behavior in high-pH gel compositions.

### Graining and Structural Development

Graining thickness, measured using calipers, increased with higher sucrose levels, confirming that high-sugar syrups promote controlled crystallization, which in turn enhances the Fast Melt texture by lowering cohesiveness.

Inner-phase solids measured by refractometry consistently increased over time, supporting the observed maturation of the texture and reduction in cohesiveness over the first several weeks.

This study demonstrated that calcium carbonate (0-6%) is an effective lever for increasing firmness, high sucrose ratios (70:30) promote low cohesiveness and ideal Fast Melt deformation behavior, and DE63 corn syrup helps maintain Aw within the desired range (<0.80) even when insoluble solids are present. Compositions combining high sucrose ratios with calcium carbonate reliably achieved the target Fast Melt texture, exhibiting hardness below 10,000 g, cohesiveness ≤0.3, and controlled water activity.

These findings illustrate how compositional levers such as insoluble solids, reducing sugar type, and syrup composition ratios may be systematically adjusted to tune hardness, cohesiveness, and water activity to produce a stable, commercially viable Fast Melt chewable gel product.

### Example 5: Stability, Efficacy, and Manufacturability of a Calcium Carbonate Fast Melt Composition

A feasibility study was conducted to evaluate the manufacturability, physical stability, active-ingredient integrity, antacid efficacy, and microbial safety of a calcium carbonate Fast Melt chewable gel composition. The study assessed the performance of a deposited gel system formulated with carrageenan as the gelling agent and containing 750 mg calcium carbonate per 4.5-gram piece. Two formulated variations of color and flavor were prepared. Pieces were produced as disk-shaped units and were manufactured at-scale under standard processing conditions.

Manufacturing was performed for a minimum duration of four hours to assess equipment compatibility, process stability, and continuous run performance at a steady state. The product was deposited, cooled, sanded with sugar, and bottled directly into two packaging configurations. Uniformity of piece weight and deposit consistency were monitored throughout production. No processing issues, clogging, or equipment interruptions were observed during the production window.

Flavor evaluations demonstrated that both flavor variants maintained their characteristic sensory profiles at all tested time points through 24 months for both packaging configurations. No off-notes, degradation odors, or undesirable flavor developments were observed. This indicates that the Fast Melt matrix effectively protected flavor components throughout shelf life.

Assay testing showed that the calcium carbonate content remained within specification throughout the entire testing period. This confirms that the high-pH active ingredient remained chemically stable and recoverable from the gel matrix, demonstrating that the Fast Melt system retains API assay across long-term storage.

Acid neutralizing capacity measurements were within specification at each time point. This demonstrates that the antacid performance of the composition is maintained throughout shelf life, confirming that the grained Fast Melt matrix does not impair the acid-neutralizing efficacy of the calcium carbonate.

Water-activity (Aw) measurements remained stable throughout shelf life, with values between 0.79 and 0.83. Although slightly above the desired 0.8 threshold, the Aw did not fluctuate over 24 months. The water-activity profile indicates that the product remains suitable for continued formulation optimization.

Microbial testing confirmed that total aerobic microorganism colony-forming unit count, yeast and mold colony-forming unit count, and specified pathogenic organism counts remained within specified microbial limits at all tested time points. The product showed no microbial growth throughout the two-year evaluation period, demonstrating that the formulation and packaging conditions provide robust microbial hurdles, even with Aw values slightly above the target threshold.

The Fast Melt calcium carbonate composition demonstrated reliable manufacturability on commercial equipment, excellent flavor and physical stability through 24 months, stable calcium carbonate potency and reliable antacid performance, consistent water-activity behavior without destabilizing fluctuations, and microbial safety within specification throughout storage.

These results confirm that the carrageenan-based Fast Melt matrix is capable of delivering a high-pH active ingredient (calcium carbonate) in a stable, efficacious, and commercially manufacturable dosage form and is suitable for large-scale production-readiness activities. Additional trials are planned to further optimize water-activity control.

### Example 6: Comparative Evaluation of Fast Melt and Non-Fast Melt Antacid Compositions

A comparative study was conducted to evaluate whether chewable gels formulated with carrageenan (Fast Melt) exhibit improved texture, disintegration, water activity control, and physical stability relative to gelatin-based gels (Non-Fast Melt), when both systems contain an identical 750 mg calcium carbonate active dose per piece. Both compositions are described in Table 14. Both compositions were produced on the same day. The higher initial pH of the carrageenan formula reflects the use of different hydrocolloids with the high-pH active system.

All products were filled into identical 8-oz polyethylene terephthalate (PET) bottles (25 pieces per bottle) and stored at ambient conditions (~72° F.) for up to three months. Testing was performed at T0, day 1, week 1, month 1, month 2, and month 3.

TABLE 14

| Each 5.2 g gel contains | | |
|---|---|---|
| 750 mg calcium | | Non-Fast Melt |
| carbonate | Fast Melt 5 | 3 |
| Water | 12-30% | 12-30% |
| Corn syrup | 10-40% | 10-40% |
| Sugar | 45-65% | 45-65% |
| Coating | 2-5% | 2-5% |
| Carrageenan | 0.5-2.5% | N/A |
| Calcium carbonate | 10-20% | 10-20% |
| Gelatin | NA | 3-10% |
| Flavor | 0.1-1% | 0.1-1% |
| Initial pH | 8.69 | 7.72 |

A comparative evaluation was performed to measure the changes in water activity, hardness, and cohesiveness for two compositions.

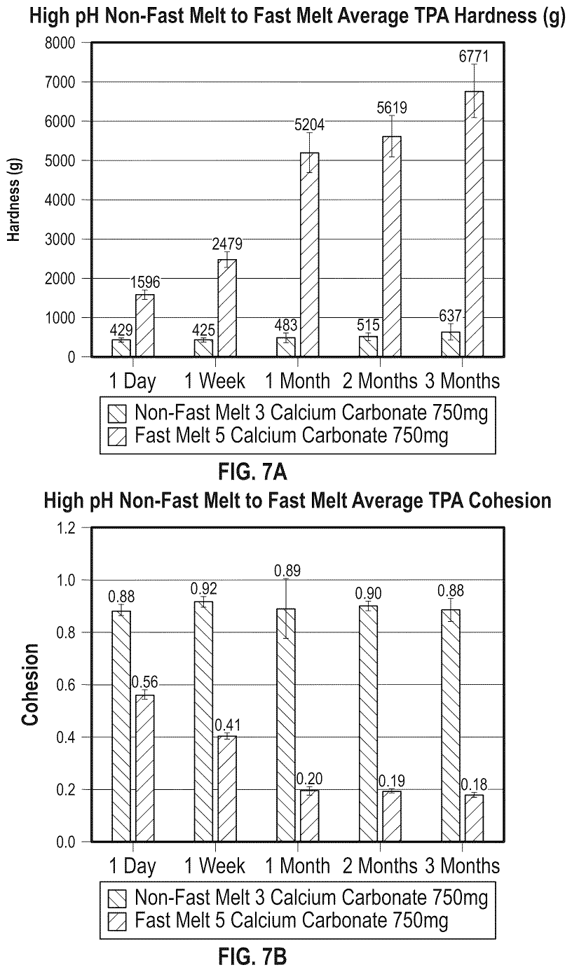

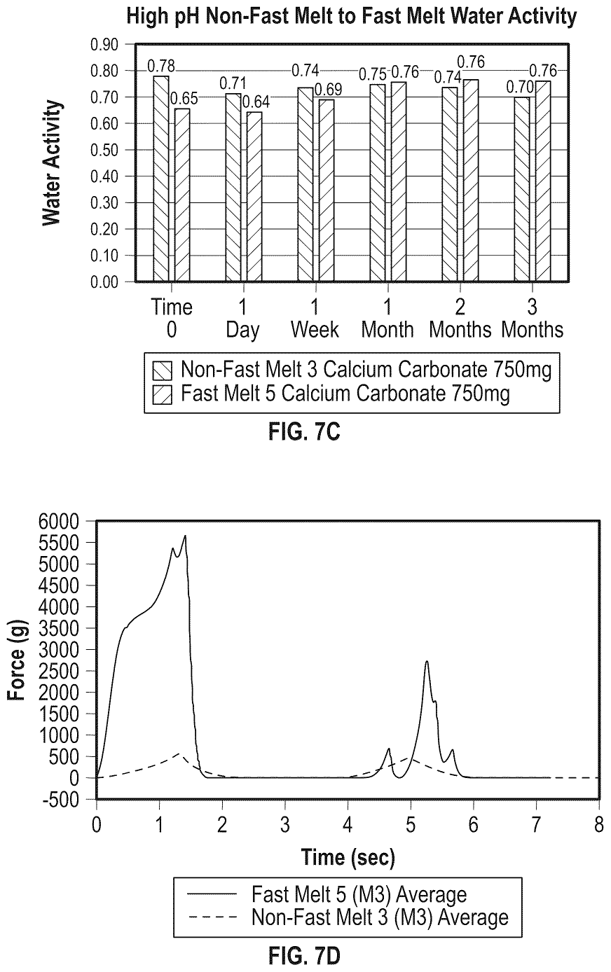

As shown in FIG. 7C, water activity remained similar for both compositions across all tested timepoints (T0, day 1, week 1, month 1, month 2, month 3). Non-Fast Melt 3 ranged from approximately 0.65-0.76. Fast Melt 5 ranged from approximately 0.69-0.76. Despite minor fluctuations, both systems maintained Aw values within a comparable range. The similar water-activity profiles demonstrate that the difference in gelling systems (gelatin vs. carrageenan) does not significantly impact Aw under the storage conditions tested.

As shown in FIGS. 7A, both compositions demonstrated an increase in hardness over the three-month storage period; however, Fast Melt 5 exhibited substantially higher hardness values at every timepoint. Table 15 shows the representative hardness values. These results confirm that carrageenan-based Fast Melt compositions develop a firmer, more structured outer matrix as they age, consistent with crystalline (“grained”) Fast Melt texture formation. Gelatin-based gels exhibit only modest increases in hardness over time.

TABLE 15

| Timepoint | Non-Fast Melt 3 | Fast Melt 5 |
|---|---|---|
| Day 1 | ~429 g | ~1596 g |
| 1 Week | ~425 g | ~2479 g |
| 1 Month | ~483 g | ~5204 g |
| 2 Months | ~515 g | ~5619 g |
| 3 Months | ~637 g | ~6771 g |

Cohesiveness trends revealed a divergence between the two systems. As seen in FIG. 7B, Fast Melt 5 exhibited a steady and pronounced decrease in cohesiveness over the first month and continuing through month 3 (from ~0.56 at day 1 to ~0.18 at month 3). Non-Fast Melt 3 remained cohesive, with values near 0.88-0.92 throughout the entire 3-month period. This decreasing cohesiveness in the Fast Melt compositions is consistent with the formation of a brittle, rapidly disintegrating Fast Melt matrix, whereas the gelatin system retains its characteristic elasticity and cohesiveness.

As shown in FIG. 7D, at the Month 3 (M3) timepoint, Texture Profile Analysis (TPA) force-time curves revealed clear mechanical distinctions between the Fast Melt and Non-Fast Melt compositions. The Fast Melt 5 composition exhibited a substantially higher first compression peak force, exceeding approximately 5,000 g, along with visible fracture features during compression, indicative of a firm, grained outer structure that fractures readily under load. The second compression peak for Fast Melt 5 was markedly reduced, demonstrating low recovery, low cohesiveness, and limited resilience after deformation. In contrast, the Non-Fast Melt 3 composition exhibited a significantly lower first compression peak force and a more gradual force response, followed by a more pronounced second compression peak, reflecting higher elasticity and cohesiveness typical of gelatin-based gels. These results confirm that, after aging, the Fast Melt composition develops a high-hardness, low-cohesion mechanical profile consistent with rapid breakdown during mastication, whereas the Non-Fast Melt composition retains a softer, more elastic texture that does not exhibit fast-melt behavior.

The combined water-activity and TPA data demonstrate: 1) water activity remains similar between the two high-pH gel systems over time; 2) Fast Melt 5 develops significantly greater hardness than Non-Fast Melt 3, consistent with structured graining and Fast Melt texture; 3) Fast Melt 5 shows substantial reductions in cohesiveness, producing a brittle, low-resilience texture ideal for rapid breakdown in the mouth; and 4) Non-Fast Melt 3 (gelatin) maintains higher cohesiveness and lower hardness, behaviors typical of traditional viscoelastic gummies.

These results further confirm that carrageenan-based Fast Melt gels provide a distinct and superior Fast Melt texture profile that cannot be achieved using conventional gelatin formulations standard to the industry under identical storage and formulation conditions.

Disintegration testing was conducted. The study was performed following the USP<701> Disintegration method, using purified water as the test medium, with pieces being cut in half. Test samples consisted of calcium carbonate chewable gels prepared with either a carrageenan hydrocolloid system or a gelatin hydrocolloid system.

The Fast Melt gummies exhibited a mean disintegration time of 25 minutes 14 seconds, whereas the conventional gelatin-based gummies exhibited a mean disintegration time of 35 minutes 48 seconds. These results demonstrate that Fast Melt chewable gels disintegrate more rapidly under USP<701> conditions compared to conventional gelatin-based systems. The data indicate that Fast Melt formulations provide a more efficient disintegration profile for calcium carbonate gummies and may be preferred in formulations requiring faster in-vitro disintegration.

1. A chewable composition comprising:
primary ingredient selected from the group consisting of an active pharmaceutical ingredient, a dietary supplement, a food, and any combination thereof, wherein the primary ingredient is present in the chewable composition in an amount from about 0.05% to about 35% by weight;
a hydrocolloid present in the chewable composition in an amount from about 0.5% to about 15% by weight;
a bulk matrix present in the chewable composition in an amount from about 65% to about 99% by weight, wherein the bulk matrix composition includes grained particles; and
water,
wherein the chewable composition has a Fast Melt texture.

2. The chewable composition of claim 1, wherein the primary ingredient, the hydrocolloid, the bulk matrix, and the water are uniformly distributed in the chewable composition.

3. The chewable composition of claim 1, wherein the primary ingredient includes an active pharmaceutical ingredient.

4. The chewable composition of claim 3, wherein the active pharmaceutical ingredient includes an antacid, laxative, stomach relief medication, antiflatulent, antidiarrheal, antihistamine, allergy medication, antibiotic, protein, peptide, antitussive, or any combination thereof.

5. The chewable composition of claim 3 wherein the active pharmaceutical ingredient includes calcium carbonate, bismuth subsalicylate, dextromethorphan hydrobromide, doxylamine succinate, or any combination thereof.

6. The chewable composition of claim 1, wherein the hydrocolloid comprises carrageenan, gelatin, pectin, agar, starch, locust bean gum, konjac, gum Arabic, sago, or any combination thereof.

7. The chewable composition of claim 6, wherein the hydrocolloid comprises carrageenan.

8. The chewable composition of claim 1, wherein the bulk matrix includes a sugar.

9. The chewable composition of claim 1, wherein the bulk matrix includes a reducing sugar, a non-reducing sugar, or a combination thereof.

10. The chewable composition of claim 9, wherein the bulk matrix includes a reducing sugar and a non-reducing sugar, wherein the mass ratio of the non-reducing sugar to the reducing sugar ranges from 90:10 to 30:70.

11. The chewable composition of claim 9, wherein the reducing sugar is selected from the group consisting of glucose, fructose, galactose, psicose, isomaltulose, tagatose, galactose, trehalose, and any combination thereof.

12. The chewable composition of claim 9, wherein the non-reducing sugar is selected from the group consisting of sucrose, maltose, trehalose, sorbose, raffinose, and any combination thereof.

13. The chewable composition of claim 1, wherein the bulk matrix includes a low-calorie bulking agent.

14. The chewable composition of claim 11, wherein the low-calorie bulking agent includes a sugar alcohol.

15. The chewable composition of claim 1, further comprising a second primary ingredient.

16. The chewable composition of claim 1, further comprising an emulsifier, a solubilizing agent, a buffering agent, a stabilizing agent, a colorant, a sweetener, a preservative, a flavoring agent, a surfactant, or any combination thereof.

17. The chewable composition of claim 5, further comprising insoluble solids, and the insoluble solids comprise calcium carbonate.

18. The chewable composition of claim 1, wherein the composition has a pH of at least 5.

19. A composition for oral administration comprising:
a primary ingredient selected from the group consisting of an active pharmaceutical ingredient, a dietary supplement, a food, and any combination thereof, wherein the primary ingredient is present in the composition in an amount from about 0.05% to about 35% by weight;
a hydrocolloid present in the composition in an amount from about 0.5% to about 15% by weight;
a bulk matrix present in the composition in an amount from about 65% to about 99.5% by weight, wherein the bulk matrix includes a non-reducing sugar and a reducing sugar, and wherein the bulk matrix composition includes grained particles; and
water,
wherein the composition has a Fast Melt texture, and
wherein the mass ratio of the non-reducing sugar to the reducing sugar ranges from 90:10 to 30:70.

20. The composition of claim 14, wherein the mass ratio of the non-reducing sugar to the reducing sugar ranges from 85:15 to 60:40.
